(19) United States
(12) Patent Application Publication
(10) Pub. No.: US 2024/0084347 A1
(43) Pub. Date: Mar. 14, 2024

(54) CELLULAR PRODUCTION OF GLYCOSYLATED PRODUCTS

(71) Applicant: INBIOSE N.V., Zwijnaarde (BE)

(72) Inventors: Joeri Beauprez, Zwijnaarde (BE); Sofie De Maeseneire, Zwijnaarde (BE); Yatti De Nijs, Zwijnaarde (BE); Tom Delmulle, Zwijnaarde (BE)

(21) Appl. No.: 18/261,793

(22) PCT Filed: Jan. 17, 2022
(86) PCT No.: PCT/EP2022/050918

§ 371 Date: Jul. 17, 2023

(30) Foreign Application Priority Data
Jan. 18, 2021 (EP) 21152132.3

(51) Int. Cl. C12P 19/12; C12N 9/10; C12P 19/18

(52) U.S. Cl. CPC C12P 19/12; C12N 9/1048; C12N 9/1051; C12N 9/1081; C12N 9/1096; C12P 19/18; C12Y 204/01222; C12Y 204/99003; C12Y 204/99004; C12Y 206/01016

(57) ABSTRACT

This disclosure is in the technical field of synthetic biology and metabolic engineering. More particularly, this disclosure is in the technical field of cultivation of metabolically engineered cells. This disclosure describes a method for the production of a glycosylated product derived from UDP-GlcNAc and comprising a di- or oligosaccharide that is composed of at least two different monosaccharide subunits by a cell as well as the separation of the glycosylated product from the cultivation. Furthermore, this disclosure provides a metabolically engineered cell for production of a glycosylated product derived from UDP-GlcNAc and comprising a di- or oligosaccharide that is composed of at least two different monosaccharide subunits. This disclosure also provides a cell excreting a di- or oligosaccharide out of the cell.

### CROSS-REFERENCE TO RELATED APPLICATIONS

This application is a national phase entry under 35 U.S.C. § 371 of International Patent Application PCT/EP2022/050918, filed Jan. 17, 2022, designating the United States of America and published as International Patent Publication WO 2022/152914 A1 on Jul. 21, 2022, which claims the benefit under Article 8 of the Patent Cooperation Treaty to European Patent Application Serial No. 21152132.3, filed Jan. 18, 2021.

### STATEMENT REGARDING ELECTRONIC FILING OF A SEQUENCE LISTING

Pursuant to 37 C.F.R. § 1.821, a Sequence Listing ASCII text file entitled “033-PCT_SeqList_ST25.txt,” 332 KB in size, generated May 8, 2023, has been submitted via EFS-Web is provided in lieu of a paper copy. This Sequence Listing is hereby incorporated by reference into the specification for its disclosures.

### TECHNICAL FIELD

This disclosure is in the technical field of synthetic biology and metabolic engineering. More particularly, this disclosure is in the technical field of cultivation of metabolically engineered cells. This disclosure describes a method for the production of a glycosylated product derived from UDP-GlcNAc and comprising a di- or oligosaccharide that is composed of at least two different monosaccharide subunits by a cell as well as the separation of the glycosylated product from the cultivation. Furthermore, this disclosure provides a metabolically engineered cell for production of a glycosylated product derived from UDP-GlcNAc and comprising a di- or oligosaccharide that is composed of at least two different monosaccharide subunits. This disclosure also provides a cell excreting a di- or oligosaccharide out of the cell.

### BACKGROUND

Carbohydrates, either present as glyco-conjugated forms to proteins and lipids or as unconjugated glycans in body fluids and animal milk, are active in many biological processes including differentiation, host-pathogen interactions, and developmental and immunological reactions (Bode, Early Hum. Dev. 1-4 (2015); Reily et al., Nat. Rev. Nephrol. 15, 346-366 (2019); Varki, Glycobiology 27, 3-49 (2017)). Carbohydrates group many different structures comprising mono-, di- and oligosaccharides being built of one, two and three or more subunits, respectively. Many carbohydrates are synthesized by glycosyltransferases that transfer activated forms of monosaccharides from nucleotide-activated sugars to growing glycan chains. Nucleotide-activated sugars, also called nucleosides, relate to each monosaccharide that is substituted with a nucleotide. Nucleotide 5′-diphosphosugars (NDP-sugars including sugars linked to e.g., UDP, GDP, ADP or dTDP) represent the most common form of sugar donors used by glycosyltransferases. Nucleosides can be synthesized from NDP-pyrophosphorylase reactions, by interconversion reactions comprising epimerization/isomerization, decarboxylation, dehydration, dehydrogenation, oxidation or reduction reactions or through the salvage pathway, i.e., through breakdown of oligosaccharides (Field and Naismith, 2003, Biochemistry 42, 7637-7647; Singh et al., 2012, Nat. Prod. Rep. 29(10), 1201-1237).

The nucleoside UDP-N-acetylglucosamine (UDP-GlcNAc) is an important intermediate in the synthesis of many natural glycosylated products. Examples of such products include di- and oligosaccharides that are composed of monosaccharides comprising N-acetylglucosamine (GlcNAc), N-acetylmannosamine (ManNAc), N-acetylgalactosamine (GalNAc), 2-acetamido-2,6-dideoxy-L-arabino-4-hexulose, 2-acetamido-2,6-dideoxy-L-lyxo-4-hexulose, N-acetyl-L-rhamnosamine, N-acetyl-D-fucosamine, N-acetyl-L-pneumosamine, N-acetylmuramic acid (MurNAc), N-acetyl-L-quinovosamine, N-acetylneuraminic acid (Neu5Ac), and N-glycolylneuraminic acid (Neu5Gc) and that have widespread distribution amongst bacteria, viruses, fungi, yeast and higher organisms. Great part of the oligosaccharides derived from UDP-GlcNAc have been found in the polysaccharide structures coating the cell surfaces of bacteria or in the peptidoglycan (or murein) layer in the periplasmic space of almost all bacteria. Lipopolysaccharides (LPS) are a major component of the outer membrane of Gram-negative bacteria. While LPS are protecting the bacterial membrane from certain kinds of chemical attacks like antibiotics and detergents encountered in the gut of mammalian hosts, they also induce a strong response from animal immune systems. Due to these immunomodulatory properties, lipopolysaccharides are often of medical or veterinary interest (Bertani and Ruiz, 2018, EcoSal. Plus 8(1), ESP-0001-2018; Caroff and Karibian, 2003, Carb. Res. 338, 2431-2447; Sampath, 2018, Agriculture and Natural Resources 52(2), 115-120). Gram-positive bacteria and Archaea do not possess LPS, but have a surface layer composed of a single layer of identical proteins or glycoproteins, wherein these glycoproteins resemble the 0-antigens of Gram-negative bacteria and often contain similar sugar building blocks (Schaffer et al., 2002, J. Biol. Chem. 277(8), 6230-6239). Peptidoglycan (or murein) is a glycosidic structure being composed of alternating GlcNAc and MurNAc sugars that are cross-linked by short peptide bridges, which maintains the osmotic pressure and cell structure of the bacteria (Vollmer et al., 2008, FEMS Microb. Rev. 32(2), 149-167). Eukaryotes also contain a wide diversity of glycosylated products that are derived from UDP-GlcNAc. For example, oligosaccharides comprising GlcNAc, GalNAc and/or Neu5Ac building blocks are frequently identified in the milk of most mammals. Most oligosaccharides in human milk (HMOs) are unique to human milk and their composition constantly changes throughout lactation to fulfil the nutritional needs of the neonate for healthy growth and development. Studies have demonstrated HMOs support immune development, moderate intestinal permeability, influence intestinal cell responses and reduce occurrences of necrotizing enterocolitis (Walsh et al., 2020, J. Funct. Foods 72, 104052 and references therein).

There is wide interest in the synthesis of glycosylated products derived from UDP-GlcNAc, yet their industrial synthesis is to date still challenging. The synthesis involves the production of specific nucleosides and glycosyltransferases transferring the monosaccharide subunits from the nucleosides to the growing saccharide chain. A problem that occurs during cellular production of these glycosylated products is the interference with native cell wall biosynthesis routes and/or the excretion of the glycosylated products outside the cell. In addition, optimizing the carbon flow in the metabolism toward these compounds is still hampered in the production hosts. Deng and co-workers reported the successful production of GlcNAc with a recombinant E. coli cell via fermentation (Deng et al., Metab. Eng. 7, 201-214 (2005); EP1576106). However, in the microbial system, GlcNAc is being produced extracellularly of an E. coli cell, more specifically in the periplasm of E. coli, via de-phosphorylation of GlcNAc-6-phosphate during export of the latter one. As such, the GlcNAc moiety obtained is not available any longer for intracellular conversion into UDP-GlcNAc that is necessary for the production of glycosylated products derived from UDP-GlcNAc in this disclosure. Also, the process described by Deng and co-workers requires a two-phase fed batch system that needs precise control to minimize inhibitory effects onto the production host, which makes the process not of commercial interest to produce high titers of UDP-GlcNAc.

### SUMMARY OF THE DISCLOSURE

Provided are tools and methods by means of which a glycosylated product derived from UDP-GlcNAc and comprising a di- or oligosaccharide that is composed of at least two different monosaccharide subunits can be produced by a cell. Also provided are a cell and a method for the production of the glycosylated product wherein the cell is genetically modified for the production of the glycosylated product.

This disclosure provides a metabolically engineered cell and a method for the production of a glycosylated product derived from UDP-GlcNAc and comprising a di- or oligosaccharide that is composed of at least two different monosaccharide subunits. The method comprises the steps of providing a cell that expresses a variant yeast or fungal glutamine-fructose-6-phosphate aminotransferase, which synthesizes UDP-GlcNAc and which uses the UDP-GlcNAc to produce the glycosylated product, and cultivating the cell under conditions permissive to produce the glycosylated product. This disclosure also provides methods to separate the glycosylated product. Furthermore, this disclosure provides a metabolically engineered cell for production of the glycosylated product. This disclosure also provides a cell excreting a di- or oligosaccharide out of the cell.

### Definitions

The words used in this specification to describe the disclosure and its various embodiments are to be understood not only in the sense of their commonly defined meanings, but to include by special definition in this specification structure, material or acts beyond the scope of the commonly defined meanings. Thus, if an element can be understood in the context of this specification as including more than one meaning, then its use in a claim must be understood as being generic to all possible meanings supported by the specification and by the word itself.

The various embodiments and aspects of embodiments of the disclosure disclosed herein are to be understood not only in the order and context specifically described in this specification, but to include any order and any combination thereof. Whenever the context requires, all words used in the singular number shall be deemed to include the plural and vice versa. Unless defined otherwise, all technical and scientific terms used herein generally have the same meaning as commonly understood by one of ordinary skill in the art to which this disclosure belongs. Generally, the nomenclature used herein and the laboratory procedures in cell culture, molecular genetics, organic chemistry and nucleic acid chemistry and hybridization described herein are those well-known and commonly employed in the art. Standard techniques are used for nucleic acid and peptide synthesis. Generally, purification steps are performed according to the manufacturer's specifications.

In the specification, there have been disclosed embodiments of the disclosure, and although specific terms are employed, the terms are used in a descriptive sense only and not for purposes of limitation, the scope of the disclosure being set forth in the following claims. It must be understood that the illustrated embodiments have been set forth only for the purposes of example and that it should not be taken as limiting the disclosure. It will be apparent to those skilled in the art that alterations, other embodiments, improvements, details and uses can be made consistent with the letter and spirit of the disclosure herein and within the scope of this disclosure, which is limited only by the claims, construed in accordance with the patent law, including the doctrine of equivalents. In the claims that follow, reference characters used to designate claim steps are provided for convenience of description only, and are not intended to imply any particular order for performing the steps.

In this document and in its claims, the verb “to comprise” and its conjugations is used in its non-limiting sense to mean that items following the word are included, but items not specifically mentioned are not excluded. Throughout the disclosure, the verb “to comprise” may be replaced by “to consist” or “to consist essentially of” and vice versa. In addition, the verb “to consist” may be replaced by “to consist essentially of” meaning that a composition as defined herein may comprise additional component(s) than the ones specifically identified, the additional component(s) not altering the unique characteristic of the disclosure. In addition, reference to an element by the indefinite article “a” or “an” does not exclude the possibility that more than one of the elements is present, unless the context clearly requires that there be one and only one of the elements. The indefinite article “a” or “an” thus usually means “at least one.”

Throughout the disclosure, unless explicitly stated otherwise, the articles “a” and “an” are preferably replaced by “at least two,” more preferably by “at least three,” even more preferably by “at least four,” even more preferably by “at least five,” even more preferably by “at least six,” most preferably by “at least two.”

Throughout the disclosure, unless explicitly stated otherwise, the features “synthesize,” “synthesized” and “synthesis” are interchangeably used with the features “produce,” “produced” and “production,” respectively.

Each embodiment as identified herein may be combined together unless otherwise indicated. All publications, patents, and patent applications mentioned in this specification are herein incorporated by reference to the same extent as if each individual publication, patent, or patent application was specifically and individually indicated to be incorporated by reference.

According to this disclosure, the term “polynucleotide(s)” generally refers to any polyribonucleotide or polydeoxyribonucleotide, which may be unmodified RNA or DNA or modified RNA or DNA. “Polynucleotide(s)” include, without limitation, single- and double-stranded DNA, DNA that is a mixture of single- and double-stranded regions or single-, double- and triple-stranded regions, single- and double-stranded RNA, and RNA that is mixture of single- and double-stranded regions, hybrid molecules comprising DNA and RNA that may be single-stranded or, more typically, double-stranded, or triple-stranded regions, or a mixture of single- and double-stranded regions. In addition, “polynucleotide” as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide. As used herein, the term “polynucleotide(s)” also includes DNAs or RNAs as described above that contain one or more modified bases. Thus, DNAs or RNAs with backbones modified for stability or for other reasons are “polynucleotide(s)” according to this disclosure. Moreover, DNAs or RNAs comprising unusual bases, such as inosine, or modified bases, such as tritylated bases, are to be understood to be covered by the term “polynucleotides.” It will be appreciated that a great variety of modifications have been made to DNA and RNA that serve many useful purposes known to those of skill in the art. The term “polynucleotide(s)” as it is employed herein embraces such chemically, enzymatically or metabolically modified forms of polynucleotides, as well as the chemical forms of DNA and RNA characteristic of viruses and cells, including, for example, simple and complex cells. The term “polynucleotide(s)” also embraces short polynucleotides often referred to as oligonucleotide(s).

“Polypeptide(s)” refers to any peptide or protein comprising two or more amino acids joined to each other by peptide bonds or modified peptide bonds. “Polypeptide(s)” refers to both short chains, commonly referred to as peptides, oligopeptides and oligomers and to longer chains generally referred to as proteins. Polypeptides may contain amino acids other than the 20 gene encoded amino acids. “Polypeptide(s)” include those modified either by natural processes, such as processing and other post-translational modifications, but also by chemical modification techniques. Such modifications are well described in basic texts and in more detailed monographs, as well as in a voluminous research literature, and they are well known to the skilled person. The same type of modification may be present in the same or varying degree at several sites in a given polypeptide. Furthermore, a given polypeptide may contain many types of modifications. Modifications can occur anywhere in a polypeptide, including the peptide backbone, the amino acid sidechains, and the amino or carboxyl termini. Modifications include, for example, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphatidylinositol, cross-linking, cyclization, disulphide bond formation, demethylation, formation of covalent cross-links, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, lipid attachment, sulfation, gamma-carboxylation of glutamic acid residues, hydroxylation and ADP-ribosylation, selenoylation, transfer-RNA mediated addition of amino acids to proteins, such as arginylation, and ubiquitination. Polypeptides may be branched or cyclic, with or without branching. Cyclic, branched and branched circular polypeptides may result from post-translational natural processes and may be made by entirely synthetic methods, as well.

The term “polynucleotide encoding a polypeptide” as used herein encompasses polynucleotides that include a sequence encoding a polypeptide of the disclosure. The term also encompasses polynucleotides that include a single continuous region or discontinuous regions encoding the polypeptide (for example, interrupted by integrated phage or an insertion sequence or editing) together with additional regions that also may contain coding and/or non-coding sequences.

“Isolated” means altered “by the hand of man” from its natural state, i.e., if it occurs in nature, it has been changed or removed from its original environment, or both. For example, a polynucleotide or a polypeptide naturally present in a living organism is not “isolated,” but the same polynucleotide or polypeptide separated from the coexisting materials of its natural state is “isolated,” as the term is employed herein. Similarly, a “synthetic” sequence, as the term is used herein, means any sequence that has been generated synthetically and not directly isolated from a natural source. “Synthesized,” as the term is used herein, means any synthetically generated sequence and not directly isolated from a natural source.

The terms “recombinant” or “transgenic” or “metabolically engineered,” “genetically engineered” or “genetically modified,” as used herein with reference to a cell or host cell are used interchangeably and indicates that the cell replicates a heterologous nucleic acid, or expresses a peptide or protein encoded by a heterologous nucleic acid (i.e., a sequence “foreign to the cell” or a sequence “foreign to the location or environment in the cell”). Such cells are described to be transformed with at least one heterologous or exogenous gene, or are described to be transformed by the introduction of at least one heterologous or exogenous gene. Metabolically engineered or recombinant or transgenic or genetically engineered cells can contain genes that are not found within the native (non-recombinant) form of the cell. Recombinant cells can also contain genes found in the native form of the cell wherein the genes are re-introduced into the cell by artificial means. The native genes of the cell can also be modified before they are re-introduced into the recombinant cells.

The terms also encompass cells that contain a nucleic acid endogenous to the cell that has been modified or its expression or activity has been modified without removing the nucleic acid from the cell; such modifications include those obtained by gene replacement, replacement of a promoter; site-specific mutation; and related techniques. Accordingly, a “recombinant polypeptide” is one that has been produced by a recombinant cell.

The terms also encompass cells that have been modified by removing a nucleic acid endogenous to the cell by means of common well-known technologies for a skilled person (like e.g., knocking-out genes).

A “heterologous sequence” or a “heterologous nucleic acid,” as used herein, is one that originates from a source foreign to the particular cell (e.g., from a different species), or, if from the same source, is modified from its original form or place in the genome. Thus, a heterologous nucleic acid operably linked to a promoter is from a source different from that from which the promoter was derived, or, if from the same source, is modified from its original form or place in the genome. The heterologous sequence may be stably introduced, e.g., by transfection, transformation, conjugation or transduction, into the genome of the host microorganism cell, wherein techniques may be applied that will depend on the cell and the sequence that is to be introduced. Various techniques are known to a person skilled in the art and are, e.g., disclosed in Sambrook et al., Molecular Cloning: A Laboratory Manual, 2nd Ed., Cold Spring Harbor Laboratory Press, Cold Spring Harbor, N.Y. (1989). The term “mutant” or “engineered” cell or microorganism as used within the context of the present disclosure refers to a cell or microorganism that is genetically modified.

The term “endogenous,” within the context of the present disclosure refers to any polynucleotide, polypeptide or protein sequence that is a natural part of a cell and is occurring at its natural location in the cell chromosome. The term “exogenous” refers to any polynucleotide, polypeptide or protein sequence that originates from outside the cell under study and not a natural part of the cell or that is not occurring at its natural location in the cell chromosome or plasmid.

The term “heterologous” when used in reference to a polynucleotide, gene, nucleic acid, polypeptide, or enzyme refers to a polynucleotide, gene, nucleic acid, polypeptide, or enzyme that is from a source or derived from a source other than the host organism. In contrast a “homologous” polynucleotide, gene, nucleic acid, polypeptide, or enzyme is used herein to denote a polynucleotide, gene, nucleic acid, polypeptide, or enzyme that is derived from the host organism. When referring to a gene regulatory sequence or to an auxiliary nucleic acid sequence used for maintaining or manipulating a gene sequence (e.g., a promoter, a 5′ untranslated region, 3′ untranslated region, poly A addition sequence, intron sequence, splice site, ribosome binding site, internal ribosome entry sequence, genome homology region, recombination site, etc.), “heterologous” means that the regulatory sequence or auxiliary sequence is not naturally associated with the gene with which the regulatory or auxiliary nucleic acid sequence is juxtaposed in a construct, genome, chromosome, or episome. Thus, a promoter operably linked to a gene to which it is not operably linked to in its natural state (i.e., in the genome of a non-genetically engineered organism) is referred to herein as a “heterologous promoter,” even though the promoter may be derived from the same species (or, in some cases, the same organism) as the gene to which it is linked.

The term “modified activity” of a protein or an enzyme relates to a change in activity of the protein, or the enzyme compared to the wild type, i.e., natural, activity of the protein or enzyme. The modified activity can either be an abolished, impaired, reduced or delayed activity of the protein or enzyme compared to the wild type activity of the protein or the enzyme but can also be an accelerated or an enhanced activity of the protein or the enzyme compared to the wild type activity of the protein or the enzyme. A modified activity of a protein or an enzyme is obtained by modified expression of the protein or enzyme or is obtained by expression of a modified, i.e., mutant form of the protein or enzyme. A modified activity of an enzyme further relates to a modification in the apparent Michaelis constant Km and/or the apparent maximal velocity (Vmax) of the enzyme.

The term “modified expression” of a gene relates to a change in expression compared to the wild type expression of the gene in any phase of the production process of the desired compound. The modified expression is either a lower or higher expression compared to the wild type, wherein the term “higher expression” is also defined as “overexpression” of the gene in the case of an endogenous gene or “expression” in the case of a heterologous gene that is not present in the wild type strain. Lower expression or reduced expression is obtained by means of common well-known technologies for a skilled person (such as the usage of siRNA, CrispR, CrispRi, riboswitches, recombineering, homologous recombination, ssDNA mutagenesis, RNAi, miRNA, asRNA, mutating genes, knocking-out genes, transposon mutagenesis, . . . ) that are used to change the genes in such a way that they are less-able (i.e., statistically significantly ‘less-able’ compared to a functional wild-type gene) or completely unable (such as knocked-out genes) to produce functional final products. Next to changing the gene of interest in such a way that lower expression is obtained as described above, lower expression can also be obtained by changing the transcription unit, the promoter, an untranslated region, the ribosome binding site, the Shine Dalgarno sequence or the transcription terminator. Lower expression or reduced expression can be obtained, for instance, by mutating one or more base pairs in the promoter sequence or changing the promoter sequence fully to a constitutive promoter with a lower expression strength compared to the wild type or an inducible promoter that result in regulated expression or a repressible promoter that results in regulated expression. Overexpression or expression is obtained by means of common well-known technologies for a skilled person, wherein the gene is part of an “expression cassette” that relates to any sequence in which a promoter sequence, untranslated region sequence (containing either a ribosome binding sequence or Shine Dalgarno sequence), a coding sequence and optionally a transcription terminator is present and leading to the expression of a functional active protein. The expression is either constitutive or regulated.

The term “constitutive expression” is defined as expression that is not regulated by transcription factors other than the subunits of the RNA polymerase (comprising bacterial sigma factors like s⁷⁰, s⁵⁴, or related s-factors and the yeast mitochondrial RNA polymerase specificity factor MTF1 that co-associates with the RNA polymerase core enzyme) under certain growth conditions. Non-limiting examples of such transcription factors are CRP, LacI, ArcA, Cra, IclR in E. coli, or Aft2p, Crz1p, Skn7 in Saccharomyces cerevisiae, or, DeoR, GntR, Fur in B. subtilis. The RNA polymerase is the catalytic machinery for the synthesis of RNA from a DNA template. RNA polymerase binds a specific DNA sequence to initiate transcription, for instance, via a sigma factor in prokaryotic hosts or via MTF1 in yeasts. Constitutive expression offers a constant level of expression with no need for induction or repression.

The term “conditional expression upon non-chemical induction or repression” is defined as a facultative or regulatory or tuneable expression of a gene that is only expressed upon a certain natural condition of the host (e.g., mating phase of budding yeast, stationary phase of bacteria, organism being in labor, or during lactation), as a response to an environmental change (e.g., anaerobic or aerobic growth, oxidative stress, temperature changes like e.g., heat-shock or cold-shock, osmolarity, light conditions) or dependent on the position of the developmental stage or the cell cycle of the host cell including but not limited to apoptosis and autophagy. Conditional expression allows for control as to when a gene is expressed. “Non-chemical induction or repression” is defined as tuneable expression that is not related to the presence or absence of a chemical compound. The term “chemical compound” refers to molecules comprising carbon sources (comprising glucose, allo-lactose, lactose, galactose, glycerol, arabinose), alcohols (comprising methanol, ethanol), acidic compounds (like acetate, formate), IPTG, metal ions (comprising aluminum, copper, zinc, nitrogen), phosphates, aromates (like xylene). Starvation and nitrogen depletion are to be understood as chemical repression resulting in conditional expression.

The term “control sequences” refers to sequences recognized by the cells transcriptional and translational systems, allowing transcription and translation of a polynucleotide sequence to a polypeptide. Such DNA sequences are thus necessary for the expression of an operably linked coding sequence in a particular cell or organism. Such control sequences can be, but are not limited to, promoter sequences, ribosome binding sequences, Shine Dalgarno sequences, Kozak sequences, transcription terminator sequences. The control sequences that are suitable for prokaryotes, for example, include a promoter, optionally an operator sequence, and a ribosome binding site. Eukaryotic cells are known to utilize promoters, polyadenylation signals, and enhancers. DNA for a presequence or secretory leader may be operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it affects the transcription of the sequence; or a ribosome binding site is operably linked to a coding sequence if it is positioned so as to facilitate translation. The control sequences can furthermore be controlled with external chemicals, such as, but not limited to, IPTG, arabinose, lactose, allo-lactose, rhamnose or fucose via an inducible promoter or via a genetic circuit that either induces or represses the transcription or translation of the polynucleotide to a polypeptide.

Generally, “operably linked” means that the DNA sequences being linked are contiguous, and, in the case of a secretory leader, contiguous and in reading phase. However, enhancers do not have to be contiguous.

The term “wild type” refers to the commonly known genetic or phenotypical situation as it occurs in nature.

The term “modified expression of a protein” as used herein refers to i) higher expression or overexpression of an endogenous protein, ii) expression of a heterologous protein or iii) expression and/or overexpression of a variant protein that has a higher activity compared to the wild-type (i.e., native) protein.

As used herein, the term “mammary cell(s)” generally refers to mammary epithelial cell(s), mammary-epithelial luminal cell(s), or mammalian epithelial alveolar cell(s), or any combination thereof. As used herein, the term “mammary-like cell(s)” generally refers to cell(s) having a phenotype/genotype similar (or substantially similar) to natural mammary cell(s) but is/are derived from non-mammary cell source(s). Such mammary-like cell(s) may be engineered to remove at least one undesired genetic component and/or to include at least one predetermined genetic construct that is typical of a mammary cell. Non-limiting examples of mammary-like cell(s) may include mammary epithelial-like cell(s), mammary epithelial luminal-like cell(s), non-mammary cell(s) that exhibits one or more characteristics of a cell of a mammary cell lineage, or any combination thereof. Further non-limiting examples of mammary-like cell(s) may include cell(s) having a phenotype similar (or substantially similar) to natural mammary cell(s), or more particularly a phenotype similar (or substantially similar) to natural mammary epithelial cell(s). A cell with a phenotype or that exhibits at least one characteristic similar to (or substantially similar to) a natural mammary cell or a mammary epithelial cell may comprise a cell (e.g., derived from a mammary cell lineage or a non-mammary cell lineage) that exhibits either naturally, or has been engineered to, be capable of expressing at least one milk component.

As used herein, the term “non-mammary cell(s)” may generally include any cell of non-mammary lineage. In the context of the disclosure, a non-mammary cell can be any mammalian cell capable of being engineered to express at least one milk component. Non-limiting examples of such non-mammary cell(s) include hepatocyte(s), blood cell(s), kidney cell(s), cord blood cell(s), epithelial cell(s), epidermal cell(s), myocyte(s), fibroblast(s), mesenchymal cell(s), or any combination thereof. In some instances, molecular biology and genome editing techniques can be engineered to eliminate, silence, or attenuate myriad genes simultaneously.

Throughout the disclosure, unless explicitly stated otherwise, the expressions “capable of . . . <verb>” and “capable to . . . <verb>” are preferably replaced with the active voice of the verb and vice versa. For example, the expression “capable of expressing” is preferably replaced with “expresses” and vice versa, i.e., “expresses” is preferably replaced with “capable of expressing.”

“Variant(s)” as the term is used herein, is a polynucleotide or polypeptide that differs from a reference polynucleotide or polypeptide, respectively, but retains essential properties. A typical variant of a polynucleotide differs in nucleotide sequence from another, reference polynucleotide. Changes in the nucleotide sequence of the variant may or may not alter the amino acid sequence of a polypeptide encoded by the reference polynucleotide. Nucleotide changes may result in amino acid substitutions, additions, deletions, fusions and truncations in the polypeptide encoded by the reference sequence, as discussed below. A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more substitutions, additions, deletions in any combination. A substituted or inserted amino acid residue may or may not be one encoded by the genetic code. A variant of a polynucleotide or polypeptide may be a naturally occurring such as an allelic variant, or it may be a variant that is not known to occur naturally. Non-naturally occurring variants of polynucleotides and polypeptides may be made by mutagenesis techniques, by direct synthesis, and by other recombinant methods known to the persons skilled in the art.

The term “derivative” of a polypeptide, as used herein, is a polypeptide that may contain deletions, additions or substitutions of amino acid residues within the amino acid sequence of the polypeptide, but that result in a silent change, thus producing a functionally equivalent polypeptide. Amino acid substitutions may be made based on similarity in polarity, charge, solubility, hydrophobicity, hydrophilicity, and/or the amphipathic nature of the residues involved. For example, nonpolar (hydrophobic) amino acids include alanine, leucine, isoleucine, valine, proline, phenylalanine, tryptophan, and methionine; planar neutral amino acids include glycine, serine, threonine, cysteine, tyrosine, asparagine, and glutamine; positively charged (basic) amino acids include arginine, lysine, and histidine; and negatively charged (acidic) amino acids include aspartic acid and glutamic acid. Within the context of this disclosure, a derivative polypeptide as used herein, refers to a polypeptide capable of exhibiting a substantially similar in vivo activity as the original polypeptide as judged by any of a number of criteria, including but not limited to enzymatic activity, and that may be differentially modified during or after translation. Furthermore, non-classical amino acids or chemical amino acid analogues can be introduced as a substitution or addition into the original polypeptide sequence.

In some embodiments, the present disclosure contemplates making functional variants by modifying the structure of an enzyme as used in this disclosure. Variants can be produced by amino acid substitution, deletion, addition, or combinations thereof. For instance, it is reasonable to expect that an isolated replacement of a leucine with an isoleucine or valine, an aspartate with a glutamate, a threonine with a serine, or a similar replacement of an amino acid with a structurally related amino acid (e.g., conservative mutations) will not have a major effect on the biological activity of the resulting molecule. Conservative replacements are those that take place within a family of amino acids that are related in their side chains. Whether a change in the amino acid sequence of a polypeptide of the disclosure results in a functional homolog can be readily determined by assessing the ability of the variant polypeptide to produce a response in cells in a fashion similar to the wild-type polypeptide.

The term “functional homolog” as used herein describes those molecules that have sequence similarity and also share at least one functional characteristic such as a biochemical activity. Functional homologs will typically give rise to the same characteristics to a similar, but not necessarily the same, degree. Functionally homologous proteins give the same characteristics where the quantitative measurement produced by one homolog is at least 10 percent of the other; more typically, at least 20 percent, between about 30 percent and about 40 percent; for example, between about 50 percent and about 60 percent; between about 70 percent and about 80 percent; or between about 90 percent and about 95 percent; between about 98 percent and about 100 percent, or greater than 100 percent of that produced by the original molecule. Thus, where the molecule has enzymatic activity, the functional homolog will have the above-recited percent enzymatic activities compared to the original enzyme. Where the molecule is a DNA-binding molecule (e.g., a polypeptide) the homolog will have the above-recited percentage of binding affinity as measured by weight of bound molecule compared to the original molecule.

A functional homolog and the reference polypeptide may be naturally occurring polypeptides, and the sequence similarity may be due to convergent or divergent evolutionary events. Functional homologs are sometimes referred to as orthologs, where “ortholog” refers to a homologous gene or protein that is the functional equivalent of the referenced gene or protein in another species.

Functional homologs can be identified by analysis of nucleotide and polypeptide sequence alignments. For example, performing a query on a database of nucleotide or polypeptide sequences can identify homologs of biomass-modulating polypeptides. Sequence analysis can involve BLAST, Reciprocal BLAST, or PSI-BLAST analysis of non-redundant databases using amino acid sequence of a biomass-modulating polypeptide as the reference sequence. Amino acid sequence is, in some instances, deduced from the nucleotide sequence. Typically, those polypeptides in the database that have greater than 40 percent sequence identity are candidates for further evaluation for suitability as a biomass-modulating polypeptide. Amino acid sequence similarity allows for conservative amino acid substitutions, such as substitution of one hydrophobic residue for another or substitution of one polar residue for another. If desired, manual inspection of such candidates can be carried out in order to narrow the number of candidates to be further evaluated. Manual inspection can be performed by selecting those candidates that appear to have domains present in productivity-modulating polypeptides, e.g., conserved functional domains.

“Fragment,” with respect to a polynucleotide, refers to a clone or any part of a polynucleotide molecule, particularly a part of a polynucleotide that retains a usable, functional characteristic of the full-length polynucleotide molecule. Useful fragments include oligonucleotides and polynucleotides that may be used in hybridization or amplification technologies or in the regulation of replication, transcription or translation. A “polynucleotide fragment” refers to any subsequence of a polynucleotide SEQ ID NO, typically, of at least about 9, 10, 11, 12 consecutive nucleotides, for example, at least about 30 nucleotides or at least about 50 nucleotides of any of the polynucleotide sequences provided herein. Exemplary fragments can additionally or alternatively include fragments that comprise, consist essentially of, or consist of a region that encodes a conserved family domain of a polypeptide. Exemplary fragments can additionally or alternatively include fragments that comprise a conserved domain of a polypeptide. As such, a fragment of a polynucleotide SEQ ID NO preferably means a nucleotide sequence that comprises or consists of the polynucleotide SEQ ID NO wherein no more than 200, 150, 100, 50 or 25 consecutive nucleotides are missing, preferably no more than 50 consecutive nucleotides are missing, and that retains a usable, functional characteristic (e.g., activity) of the full-length polynucleotide molecule that can be assessed by the skilled person through routine experimentation. Alternatively, a fragment of a polynucleotide SEQ ID NO preferably means a nucleotide sequence that comprises or consists of an amount of consecutive nucleotides from the polynucleotide SEQ ID NO and wherein the amount of consecutive nucleotides is at least 50.0%, 60.0%, 70.0%, 80.0%, 81.0%, 82.0%, 83.0%, 84.0%, 85.0%, 86.0%, 87.0%, 88.0%, 89.0%, 90.0%, 91.0%, 92.0%, 93.0%, 94.0%, 95.0%, 95.5%, 96.0%, 96.5%, 97.0%, 97.5%, 98.0%, 98.5%, 99.0%, 99.5%, 100%, preferably at least 80.0%, more preferably at least 87.0%, even more preferably at least 90.0%, even more preferably at least 95.0%, most preferably at least 97.0%, of the full-length of the polynucleotide SEQ ID NO and retains a usable, functional characteristic (e.g., activity) of the full-length polynucleotide molecule. As such, a fragment of a polynucleotide SEQ ID NO preferably means a nucleotide sequence that comprises or consists of the polynucleotide SEQ ID NO, wherein an amount of consecutive nucleotides is missing and wherein the amount is no more than 50.0%, 40.0%, 30.0% of the full-length of the polynucleotide SEQ ID NO, preferably no more than 20.0%, 15.0%, 10.0%, 9.0%, 8.0%, 7.0%, 6.0%, 5.0%, 4.5%, 4.0%, 3.5%, 3.0%, 2.5%, 2.0%, 1.5%, 1.0%, 0.5%, more preferably no more than 15.0%, even more preferably no more than 10.0%, even more preferably no more than 5.0%, most preferably no more than 2.5%, of the full-length of the polynucleotide SEQ ID NO and wherein the fragment retains a usable, functional characteristic (e.g., activity) of the full-length polynucleotide molecule that can be routinely assessed by the skilled person.

Fragments may additionally or alternatively include subsequences of polypeptides and protein molecules, or a subsequence of the polypeptide. In some cases, the fragment or domain is a subsequence of the polypeptide that performs at least one biological function of the intact polypeptide in substantially the same manner, or to a similar extent, as does the intact polypeptide. A “subsequence of the polypeptide” as defined herein refers to a sequence of contiguous amino acid residues derived from the polypeptide. For example, a polypeptide fragment can comprise a recognizable structural motif or functional domain such as a DNA-binding site or domain that binds to a DNA promoter region, an activation domain, or a domain for protein-protein interactions, and may initiate transcription. Fragments can vary in size from as few as 3 amino acid residues to the full-length of the intact polypeptide, for example, at least 10 amino acid residues in length, for example, at least about 20 amino acid residues in length, for example, at least about 30 amino acid residues in length. Preferentially a fragment is a functional fragment that has at least one property or activity of the polypeptide from which it is derived, such as, for example, the fragment can include a functional domain or conserved domain of a polypeptide. As such, a fragment of a polypeptide SEQ ID NO (or UniProt ID) preferably means a polypeptide sequence that comprises or consists of the polypeptide SEQ ID NO (or UniProt ID) wherein no more than 80, 60, 50, 40, 30, 20 or 15 consecutive amino acid residues are missing, preferably no more than 40 consecutive amino acid residues are missing, and performs at least one biological function of the intact polypeptide in substantially the same manner, preferably to a similar or greater extent, as does the intact polypeptide that can be routinely assessed by the skilled person. Alternatively, a fragment of a polypeptide SEQ ID NO (or UniProt ID) preferably means a polypeptide sequence that comprises or consists of an amount of consecutive amino acid residues from the polypeptide SEQ ID NO (or UniProt ID) and wherein the amount of consecutive amino acid residues is at least 50.0%, 60.0%, 70.0%, 80.0%, 81.0%, 82.0%, 83.0%, 84.0%, 85.0%, 86.0%, 87.0%, 88.0%, 89.0%, 90.0%, 91.0%, 92.0%, 93.0%, 94.0%, 95.0%, 95.5%, 96.0%, 96.5%, 97.0%, 97.5%, 98.0%, 98.5%, 99.0%, 99.5%, 100%, preferably at least 80.0%, more preferably at least 87.0%, even more preferably at least 90.0%, even more preferably at least 95.0%, most preferably at least 97.0% of the full-length of the polypeptide SEQ ID NO (or UniProt ID) and that performs at least one biological function of the intact polypeptide in substantially the same manner, preferably to a similar or greater extent, as does the intact polypeptide that can be routinely assessed by the skilled person. As such, a fragment of a polypeptide SEQ ID NO (or UniProt ID) preferably means a polypeptide sequence that comprises or consists of the polypeptide SEQ ID NO (or UniProt ID), wherein an amount of consecutive amino acid residues is missing and wherein the amount is no more than 50.0%, 40.0%, 30.0% of the full-length of the polypeptide SEQ ID NO (or UniProt ID), preferably no more than 20.0%, 15.0%, 10.0%, 9.0%, 8.0%, 7.0%, 6.0%, 5.0%, 4.5%, 4.0%, 3.5%, 3.0%, 2.5%, 2.0%, 1.5%, 1.0%, 0.5%, more preferably no more than 15.0%, even more preferably no more than 10.0%, even more preferably no more than 5.0%, most preferably no more than 2.5%, of the full-length of the polypeptide SEQ ID NO (or UniProt ID) and that performs at least one biological function of the intact polypeptide in substantially the same manner, preferably to a similar or greater extent, as does the intact polypeptide that can be routinely assessed by the skilled person.

Throughout the disclosure, the sequence of a polypeptide can be represented by a SEQ ID NO or alternatively by an UniProt ID. Therefore, the terms “polypeptide SEQ ID NO” and “polypeptide UniProt ID” can be interchangeably used, unless explicitly stated otherwise.

A “functional fragment” of a polypeptide has at least one property or activity of the polypeptide from which it is derived, preferably to a similar or greater extent. A functional fragment can, for example, include a functional domain or conserved domain of a polypeptide. It is understood that a polypeptide or a fragment thereof may have conservative amino acid substitutions that have substantially no effect on the polypeptide's activity. By conservative substitutions is intended substitutions of one hydrophobic amino acid for another or substitution of one polar amino acid for another or substitution of one acidic amino acid for another or substitution of one basic amino acid for another etc. Preferably, by conservative substitutions is intended combinations such as glycine by alanine and vice versa; valine, isoleucine and leucine by methionine and vice versa; aspartate by glutamate and vice versa; asparagine by glutamine and vice versa; serine by threonine and vice versa; lysine by arginine and vice versa; cysteine by methionine and vice versa; and phenylalanine and tyrosine by tryptophan and vice versa.

Homologous sequences as used herein describe those nucleotide sequences that have sequence similarity and encode polypeptides that share at least one functional characteristic such as a biochemical activity. More specifically, the term “functional homolog” as used herein describes those polypeptides that have sequence similarity (in other words, homology) and at the same time have at least one functional similarity such as a biochemical activity (Altenhoff et al., PLoS Comput. Biol. 8 (2012) e1002514).

Homologs can be identified by analysis of nucleotide and polypeptide sequence alignments. For example, performing a query on a database of nucleotide or polypeptide sequences can identify homologs of the polypeptide of interest like e.g., a biomass-modulating polypeptide, a glycosyltransferase, a protein involved in nucleotide-activated sugar synthesis or a membrane transporter protein. Sequence analysis can involve BLAST, Reciprocal BLAST, or PSI-BLAST analysis of non-redundant databases using amino acid sequence of a biomass-modulating polypeptide, a glycosyltransferase, a protein involved in nucleotide-activated sugar synthesis or a membrane transporter protein, respectively, as the reference sequence. Amino acid sequence is, in some instances, deduced from the nucleotide sequence. Typically, those polypeptides in the database that have greater than 40 percent sequence identity are candidates for further evaluation for suitability as a biomass-modulating polypeptide, a glycosyltransferase, a protein involved in nucleotide-activated sugar synthesis or a membrane transporter protein, respectively. Amino acid sequence similarity allows for conservative amino acid substitutions, such as substitution of one hydrophobic residue for another or substitution of one polar residue for another or substitution of one acidic amino acid for another or substitution of one basic amino acid for another etc. Preferably, by conservative substitutions is intended combinations such as glycine by alanine and vice versa; valine, isoleucine and leucine by methionine and vice versa; aspartate by glutamate and vice versa; asparagine by glutamine and vice versa; serine by threonine and vice versa; lysine by arginine and vice versa; cysteine by methionine and vice versa; and phenylalanine and tyrosine by tryptophan and vice versa. If desired, manual inspection of such candidates can be carried out to narrow the number of candidates to be further evaluated. Manual inspection can be performed by selecting those candidates that appear to have domains present in productivity-modulating polypeptides, e.g., conserved functional domains.

A domain can be characterized, for example, by a Pfam (El-Gebali et al., Nucleic Acids Res. 47 (2019) D427-D432), a protein fingerprint domain (PRINTS) (Attwood et al., Nucleic Acids Res. 31 (2003) 400-402), a SUBFAM domain (Gough et al., J. Mol. Biol. 313 (2001) 903-919), a TIGRFAM domain (Selengut et al., Nucleic Acids Res. 35 (2007) D260-D264), a Conserved Domain Database (CDD) (www.ncbi.nlm.nih.gov/cdd) (Lu et al., Nucleic Acids Res. 48 (2020) D265-D268) designation, a PTHR domain (www.pantherdb.org) (Mi et al., Nucleic Acids. Res. 41 (2013) D377-D386; Thomas et al., Genome Research 13 (2003) 2129-2141), an IPR (InterPro domain) (Mitchell et al., Nucleic Acids Res. 47 (2019) D351-D360) or a PATRIC identifier or PATRIC DB global family domain (www.patricbrc.org/) (Davis et al., Nucleic Acids Res. 48(D1) (2020) D606-D612).

The PANTHER (Protein ANalysis THrough Evolutionary Relationships) Classification System was designed to classify proteins (and their genes) in order to facilitate high-throughput analysis. Proteins have been classified according to: (a) Family and subfamily: families are groups of evolutionarily related proteins; subfamilies are related proteins that also have the same function; (b) Molecular function: the function of the protein by itself or with directly interacting proteins at a biochemical level, e.g., a protein kinase; (c) Biological process: the function of the protein in the context of a larger network of proteins that interact to accomplish a process at the level of the cell or organism, e.g., mitosis; (d) Pathway: similar to biological process, but a pathway also explicitly specifies the relationships between the interacting molecules.

InterPro provides functional analysis of proteins by classifying them into families and predicting domains and important sites. To classify proteins in this way, InterPro uses predictive models, known as signatures, provided by several different databases (referred to as member databases) that make up the InterPro consortium. Protein signatures from these member databases are combined into a single searchable resource, capitalizing on their individual strengths to produce a powerful integrated database and diagnostic tool.

The content of each database is fixed at each release and is not to be changed. When the content of a specific database is changed, this specific database receives a new release version with a new release date. All release versions for each database with their corresponding release dates and specific content as annotated at these specific release dates are available and known to those skilled in the art. The PANTHER (PTHR) database (www.pantherdb.org) used herein was PANTHER 15.0 released on 2020 Sep. 10. The InterPro database (ebi.ac.uk/interpro) used herein was InterPro 82.0 released on 2020 Oct. 8.

Protein or polypeptide sequence information and functional information can be provided by a comprehensive resource for protein sequence and annotation data like e.g., the Universal Protein Resource (UniProt) (www.uniprot.org) (Nucleic Acids Res. 2021, 49(D1), D480-D489). UniProt comprises the expertly and richly curated protein database called the UniProt Knowledgebase (UniProtKB), together with the UniProt Reference Clusters (UniRef) and the UniProt Archive (UniParc). The UniProt identifiers (UniProt ID) are unique for each protein present in the database.

The terms “identical” or “percent identity” or “% identity” in the context of two or more nucleic acid or polypeptide sequences, refer to two or more sequences or subsequences that are the same or have a specified percentage of amino acid residues or nucleotides that are the same, when compared and aligned for maximum correspondence, as measured using sequence comparison algorithms or by visual inspection. For sequence comparison, one sequence acts as a reference sequence, to which test sequences are compared. When using a sequence comparison algorithm, test and reference sequences are inputted into a computer, subsequence coordinates are designated, if necessary, and sequence algorithm program parameters are designated. The sequence comparison algorithm then calculates the percent sequence identity for the test sequence(s) relative to the reference sequence, based on the designated program parameters. The percentage of sequence identity can be, preferably is, determined by alignment of the two sequences and identification of the number of positions with identical residues divided by the number of residues in the shorter of the sequences×100. Percent identity may be calculated globally over the full-length sequence of a given SEQ ID NO, i.e., the reference sequence, resulting in a global percent identity score. Alternatively, percent identity may be calculated over a partial sequence of the reference sequence, resulting in a local percent identity score. A partial sequence preferably means at least 50%, 60%, 70%, 80%, 90% or 95% of the full-length reference sequence. In another preferred embodiment, a partial sequence of a reference polypeptide sequence means a stretch of at least 200 amino acid residues up to the total number of amino acid residues of a reference polypeptide sequence. Using the full-length of the reference sequence in a local sequence alignment results in a global percent identity score between the test and the reference sequence. Percent identity can be determined using different algorithms like, for example, BLAST and PSI-BLAST (Altschul et al., 1990, J Mol Biol 215:3, 403-410; Altschul et al., 1997, Nucleic Acids Res 25: 17, 3389-402), the Clustal Omega method (Sievers et al., 2011, Mol. Syst. Biol. 7:539), the MatGAT method (Campanella et al., 2003, BMC Bioinformatics, 4:29) or EMBOSS Needle (galaxy-iuc.github.io/emboss-5.0-docs/needle.html).

The BLAST (Basic Local Alignment Search Tool)) method of alignment is an algorithm provided by the National Center for Biotechnology Information (NCBI) to compare sequences using default parameters. The program compares nucleotide or protein sequences to sequence databases and calculates the statistical significance. PSI-BLAST (Position-Specific Iterative Basic Local Alignment Search Tool) derives a position-specific scoring matrix (PSSM) or profile from the multiple sequence alignment of sequences detected above a given score threshold using protein-protein BLAST (BLASTp). The BLAST method can be used for pairwise or multiple sequence alignments. Pairwise Sequence Alignment is used to identify regions of similarity that may indicate functional, structural and/or evolutionary relationships between two biological sequences (protein or nucleic acid). The web interface for BLAST is available at: blast.ncbi.nlm.nih.gov/Blast.cgi.

Clustal Omega (Clustal W) is a multiple sequence alignment program that uses seeded guide trees and HMI profile-profile techniques to generate alignments between three or more sequences. It produces biologically meaningful multiple sequence alignments of divergent sequences. The web interface for Clustal W is available at www.ebi.ac.uk/Tools/msa/clustalo/. Default parameters for multiple sequence alignments and calculation of percent identity of protein sequences using the Clustal W method are: enabling de-alignment of input sequences: FALSE; enabling mbed-like clustering guide-tree: TRUE; enabling mbed-like clustering iteration: TRUE; Number of (combined guide-tree/HMIM) iterations: default(0); Max Guide Tree Iterations: default [−1]; Max HMM Iterations: default [−1]; order: aligned.

MatGAT (Matrix Global Alignment Tool) is a computer application that generates similarity/identity matrices for DNA or protein sequences without needing pre-alignment of the data. The program performs a series of pairwise alignments using the Myers and Miller global alignment algorithm, calculates similarity and identity, and then places the results in a distance matrix. The user may specify which type of alignment matrix (e.g., BLOSUM50, BLOSUM62, and PAM250) to employ with their protein sequence examination.

EMBOSS Needle (galaxy-iuc.gibhub.io/emboss-5.0-docs/needle.html) uses the Needleman-Wunsch global alignment algorithm to find the optimal alignment (including gaps) of two sequences when considering their entire length. The optimal alignment is ensured by dynamic programming methods by exploring all possible alignments and choosing the best. The Needleman-Wunsch algorithm is a member of the class of algorithms that can calculate the best score and alignment in the order of mn steps, (where ‘n’ and ‘m’ are the lengths of the two sequences). The gap open penalty (default 10.0) is the score taken away when a gap is created. The default value assumes you are using the EBLOSUM62 matrix for protein sequences. The gap extension (default 0.5) penalty is added to the standard gap penalty for each base or residue in the gap. This is how long gaps are penalized.

For the purposes of this disclosure, percent identity is determined using MatGAT2.01 (Campanella et al., 2003, BMC Bioinformatics 4:29). The following default parameters for protein are employed: (1) Gap cost Existence: 12 and Extension: 2; (2) The Matrix employed was BLOSUM65.

The term “glutamine-fructose-6-phosphate aminotransferase” as used herein refers to an enzyme that catalyzes the conversion of D-fructose-6-phosphate+L-glutamine into D-glucosamine-6-phosphate+L-glutamate. Glutamine-fructose-6-phosphate aminotransferase is the first and rate-limiting enzyme of the hexosamine biosynthetic pathway. The final product of the hexosamine pathway is UDP-N-acetylglucosamine or UDP-GlcNAc. A glutamine-fructose-6-phosphate aminotransferase is characterized by the presence of the PTHR10937 domain as annotated in the PANTHER Classification System (www.pantherdb.org) or by presence of the IPR005855 domain as annotated in the InterPro Classification System (www.ebi.ac.uk/interpro/entry/InterPro/IPR005855/). Alternative names for this type of enzyme comprise glutamine-fructose-6-phosphate transaminase (isomerizing), D-fructose-6-phosphate amidotransferase, GlcN6P synthase, glucosamine 6-phosphate synthase, glucosamine-6-phosphate isomerase (glutamine-forming), glucosaminephosphate isomerase, hexosephosphate aminotransferase, glucosamine-6-phosphate synthase, L-glutamine-D-fructose-6-phosphate amidotransferase and L-glutamine:D-fructose-6-phosphate isomerase (deaminating). The terms “GFA1” and “GFAT” as used herein are used interchangeably and refer to the yeast and fungal glutamine-fructose-6-phosphate aminotransferase as found in, for example, S. cerevisiae, Candida albicans, Schizosaccharomyces pombe and Sporothrix schenckii. The term “glmS” is an analog of GFA1 and refers to the prokaryotic glutamine-fructose-6-phosphate aminotransferase as found in, for example, E. coli K-12 strains, E. coli O6:H1, E. coli O157:H7, Pasteurella multocida and Neisseria meningitidis serogroup B (strain MC58).

A glucosamine 6-phosphate N-acetyltransferase is an enzyme that catalyzes the transfer of an acetyl group from acetyl-CoA to D-glucosamine-6-phosphate thereby generating a free CoA and N-acetyl-D-glucosamine 6-phosphate. Alternative names comprise aminodeoxyglucosephosphate acetyltransferase, D-glucosamine-6-P N-acetyltransferase, glucosamine 6-phosphate acetylase, glucosamine 6-phosphate N-acetyltransferase, glucosamine-6-phosphate acetylase, N-acetylglucosamine-6-phosphate synthase, phosphoglucosamine acetylase, phosphoglucosamine N-acetylase, phosphoglucosamine transacetylase, GNA and GNA1. A phosphoacetylglucosamine mutase is an enzyme that catalyzes the conversion of N-acetyl-D-glucosamine 6-phosphate into N-acetyl-D-glucosamine-1-phosphate. Alternative names comprise PAGM, acetylglucosamine phosphomutase, N-acetylglucosamine-phosphate mutase and PGM-complementing protein 1. A UDP-N-acetylglucosamine pyrophosphorylase is an enzyme involved in the synthesis of UDP-N-acetyl-D-glucosamine from N-acetyl-D-glucosamine 1-phosphate. A galactoside beta-1,3-N-acetylglucosaminyltransferase is an enzyme that catalyzes the transfer of UDP-GlcNAc to a galactose unit in a beta 1,3 linkage. A UTP-glucose-1-phosphate uridylyltransferase is an enzyme that synthesizes UDP-glucose from glucose-1-phosphate and UTP. Alternative names comprise alpha-D-glucosyl-1-phosphate uridylyltransferase, UDP-glucose pyrophosphorylase, UDPGP and uridine diphosphoglucose pyrophosphorylase. A UDP-glucose 4-epimerase is an enzyme that catalyzes the reversible conversion of UDP-glucose to UDP-galactose. Alternative names comprise galactowaldenase and UDP-galactose 4-epimerase. An N-acetylglucosamine beta-1,3-galactosyltransferase is an enzyme that catalyzes the transfer of a galactose from a UDP-galactose donor to a terminal GlcNAc residue in a glycan chain in a beta 1,3-linkage. An N-acetylglucosamine beta-1,4-galactosyltransferase is an enzyme that catalyzes the transfer of a galactose from a UDP-galactose donor to a terminal GlcNAc residue in a glycan chain in a beta 1,4-linkage. Lactose permease is a membrane protein that facilitates the passage of lactose. A UDP-N-acetylglucosamine 2-epimerase or a UDP-2-acetamido-2,6-dideoxy-L-talose 2-epimerase is an enzyme that catalyzes the reversible conversion of UDP-N-acetylglucosamine (UDP-GlcNAc) to UDP-N-acetylmannosamine (UDP-ManNAc). An N-acetylneuraminate synthase or N-acetylneuraminic acid synthase is an enzyme that catalyzes the conversion of N-acetylmannosamine (ManNAc) to N-acetylneuraminate (or sialic acid, Neu5Ac). An N-acylneuraminate cytidylyltransferase is an enzyme that catalyzes the conversion of N-acetylneuraminate to CMP-N-acetylneuraminic acid or CMP-Neu5Ac. Alternative names comprise CMP-N-acetylneuraminic acid synthase and CMP-NeuNAc synthase. A glucose-6-phosphate isomerase is an enzyme that catalyzes the interconversion of glucose-6-phosphate and fructose-6-phosphate. Alternative names comprise phosphoglucose isomerase/phosphoglucoisomerase (PGI) and phosphohexose isomerase (PHI).

The term “glycosyltransferase” as used herein refers to an enzyme capable to catalyze the transfer of sugar moieties from activated donor molecules to specific acceptor molecules, forming glycosidic bonds. The as such synthesized oligosaccharides can be of the linear type or of the branched type and can contain multiple monosaccharide building blocks. A classification of glycosyltransferases using nucleotide diphospho-sugar, nucleotide monophospho-sugar and sugar phosphates and related proteins into distinct sequence-based families has been described (Campbell et al., Biochem. J. 326, 929-939 (1997)) and is available on the CAZy (CArbohydrate-Active EnZymes) website (www.cazy.org).

As used herein the glycosyltransferase can be selected from the list comprising but not limited to: fucosyltransferases, sialyltransferases, galactosyltransferases, glucosyltransferases, mannosyltransferases, N-acetylglucosaminyltransferases, N-acetylgalactosaminyltransferases, N-acetylmannosaminyltransferases, xylosyltransferases, glucuronyltransferases, galacturonyltransferases, glucosaminyltransferases, N-glycolylneuraminyltransferases, rhamnosyltransferases, N-acetylrhamnosyltransferases, UDP-4-amino-4,6-dideoxy-N-acetyl-beta-L-altrosamine transaminases and fucosaminyltransferases.

Fucosyltransferases are glycosyltransferases that transfer a fucose residue (Fuc) from a GDP-fucose (GDP-Fuc) donor onto a glycan acceptor. Fucosyltransferases comprise alpha-1,2-fucosyltransferases, alpha-1,3-fucosyltransferases, alpha-1,3/4-fucosyltransferases, alpha-1,4-fucosyltransferases and alpha-1,6-fucosyltransferases that catalyze the transfer of a Fuc residue from GDP-Fuc onto a glycan acceptor via alpha-glycosidic bonds.

Fucosyltransferases can be found but are not limited to the GT10, GT11, GT23, GT65 and GT68 CAZy families. Sialyltransferases are glycosyltransferases that transfer a sialyl group (like Neu5Ac or Neu5Gc) from a donor (like CMP-Neu5Ac or CMP-Neu5Gc) onto a glycan acceptor. Sialyltransferases comprise alpha-2,3-sialyltransferases, alpha-2,6-sialyltransferases and alpha-2,8-sialyltransferases that catalyze the transfer of a sialyl group onto a glycan acceptor via alpha-glycosidic bonds. Sialyltransferases can be found but are not limited to the GT29, GT42, GT80 and GT97 CAZy families. Galactosyltransferases are glycosyltransferases that transfer a galactosyl group (Gal) from a UDP-galactose (UDP-Gal) donor onto a glycan acceptor. Galactosyltransferases comprise beta-1,3-galactosyltransferases, N-acetylglucosamine beta-1,3-galactosyltransferases, beta-1,4-galactosyltransferases, N-acetylglucosamine beta-1,4-galactosyltransferases, alpha-1,3-galactosyltransferases and alpha-1,4-galactosyltransferases that transfer a Gal residue from UDP-Gal onto a glycan acceptor via alpha- or beta-glycosidic bonds. Galactosyltransferases can be found but are not limited to the GT2, GT6, GT8, GT25 and GT92 CAZy families. Glucosyltransferases are glycosyltransferases that transfer a glucosyl group (Glc) from a UDP-glucose (UDP-Glc) donor onto a glycan acceptor. Glucosyltransferases comprise alpha-glucosyltransferases, beta-1,2-glucosyltransferases, beta-1,3-glucosyltransferases and beta-1,4-glucosyltransferases that transfer a Glc residue from UDP-Glc onto a glycan acceptor via alpha- or beta-glycosidic bonds. Glucosyltransferases can be found but are not limited to the GT1, GT4 and GT25 CAZy families. Mannosyltransferases are glycosyltransferases that transfer a mannose group (Man) from a GDP-mannose (GDP-Man) donor onto a glycan acceptor. Mannosyltransferases comprise alpha-1,2-mannosyltransferases, alpha-1,3-mannosyltransferases and alpha-1,6-mannosyltransferases that transfer a Man residue from GDP-Man onto a glycan acceptor via alpha-glycosidic bonds. Mannosyltransferases can be found but are not limited to the GT22, GT39, GT62 and GT69 CAZy families. N-acetylglucosaminyltransferases are glycosyltransferases that transfer an N-acetylglucosamine group (GlcNAc) from a UDP-N-acetylglucosamine (UDP-GlcNAc) donor onto a glycan acceptor. N-acetylglucosaminyltransferases can be found but are not limited to GT2 and GT4 CAZy families. Galactoside beta-1,3-N-acetylglucosaminyltransferases are part of N-acetylglucosaminyltransferases and transfer GlcNAc from a UDP-GlcNAc donor onto a terminal galactose unit present in a substrate via a beta-1,3-linkage. Beta-1,6-N-acetylglucosaminyltransferases are N-acetylglucosaminyltransferases that transfer GlcNAc from a UDP-GlcNAc donor onto a substrate via a beta-1,6-linkage. N-acetylgalactosaminyltransferases are glycosyltransferases that transfer an N-acetylgalactosamine group (GalNAc) from a UDP-N-acetylgalactosamine (UDP-GalNAc) donor onto a glycan acceptor. N-acetylgalactosaminyltransferases can be found but are not limited to GT7, GT12 and GT27 CAZy families. Alpha-1,3-N-acetylgalactosaminyltransferases are part of the N-acetylgalactosaminyltransferases and transfer GalNAc from a UDP-GalNAc donor to a substrate via an alpha-1,3-linkage. N-acetylmannosaminyltransferases are glycosyltransferases that transfer an N-acetylmannosamine group (ManNAc) from a UDP-N-acetylmannosamine (UDP-ManNAc) donor onto a glycan acceptor. Xylosyltransferases are glycosyltransferases that transfer a xylose residue (Xyl) from a UDP-xylose (UDP-Xyl) donor onto a glycan acceptor. Xylosyltransferases can be found but are not limited to GT14, GT61 and GT77 CAZy families. Glucuronyltransferases are glycosyltransferases that transfer a glucuronate from a UDP-glucuronate donor onto a glycan acceptor via alpha- or beta-glycosidic bonds. Glucuronyltransferases can be found but are not limited to GT4, GT43 and GT93 CAZy families. Galacturonyltransferases are glycosyltransferases that transfer a galacturonate from a UDP-galacturonate donor onto a glycan acceptor. N-glycolylneuraminyltransferases are glycosyltransferases that transfer an N-glycolylneuraminic acid group (Neu5Gc) from a CMP-Neu5Gc donor onto a glycan acceptor. Rhamnosyltransferases are glycosyltransferases that transfer a rhamnose residue from a GDP-rhamnose donor onto a glycan acceptor. Rhamnosyltransferases can be found but are not limited to the GT1, GT2 and GT102 CAZy families. N-acetylrhamnosyltransferases are glycosyltransferases that transfer an N-acetylrhamnosamine residue from a UDP-N-acetyl-L-rhamnosamine donor onto a glycan acceptor. UDP-4-amino-4,6-dideoxy-N-acetyl-beta-L-altrosamine transaminases are glycosyltransferases that use a UDP-2-acetamido-2,6-dideoxy-L-arabino-4-hexulose in the biosynthesis of pseudaminic acid, which is a sialic acid-like sugar that is used to modify flagellin. UDP-N-acetylglucosamine enolpyruvyl transferases (murA) are glycosyltransferases that transfer an enolpyruvyl group from phosphoenolpyruvate (PEP) to UDP-N-acetylglucosamine (UDPAG) to form UDP-N-acetylglucosamine enolpyruvate. Fucosaminyltransferases are glycosyltransferases that transfer an N-acetylfucosamine residue from a dTDP-N-acetylfucosamine or a UDP-N-acetylfucosamine donor onto a glycan acceptor.

The terms “nucleotide-sugar,” “activated sugar,” “nucleoside,” “activated monosaccharide,” “nucleotide-activated sugar” or “nucleotide donor” are used herein interchangeably and refer to activated forms of monosaccharides. Examples of activated monosaccharides include but are not limited to UDP-N-acetylglucosamine (UDP-GlcNAc), UDP-N-acetylgalactosamine (UDP-GalNAc), UDP-N-acetylmannosamine (UDP-ManNAc), UDP-glucose (UDP-Glc), UDP-galactose (UDP-Gal), GDP-mannose (GDP-Man), UDP-glucuronate, UDP-galacturonate, UDP-2-acetamido-2,6-dideoxy-L-arabino-4-hexulose, UDP-2-acetamido-2,6-dideoxy-L-lyxo-4-hexulose, UDP-N-acetyl-L-rhamnosamine (UDP-L-RhaNAc or UDP-2-acetamido-2,6-dideoxy-L-mannose), dTDP-N-acetylfucosamine, UDP-N-acetylfucosamine (UDP-L-FucNAc or UDP-2-acetamido-2,6-dideoxy-L-galactose), UDP-N-acetyl-L-pneumosamine (UDP-L-PneNAC or UDP-2-acetamido-2,6-dideoxy-L-talose), UDP-N-acetylmuramic acid, UDP-N-acetyl-L-quinovosamine (UDP-L-QuiNAc or UDP-2-acetamido-2,6-dideoxy-L-glucose), GDP-L-quinovose, CMP-sialic acid (CMP-Neu5Ac or CMP-N-acetylneuraminic acid), CMP-Neu4Ac, CMP-Neu5Ac9N3, CMP-Neu4,5AC2, CMP-Neu5,7Ac2, CMP-Neu5,9Ac2, CMP-Neu5,7(8,9)Ac2, CMP-N-glycolylneuraminic acid (CMP-Neu5Gc), GDP-fucose (GDP-Fuc), GDP-rhamnose and UDP-xylose. Nucleotide-sugars act as glycosyl donors in glycosylation reactions. Glycosylation reactions are reactions that are catalyzed by glycosyltransferases.

The term “monosaccharide” as used herein refers to a sugar that is not decomposable into simpler sugars by hydrolysis, is classed either an aldose or ketose, and contains one or more hydroxyl groups per molecule. Monosaccharides are saccharides containing only one simple sugar. Examples of monosaccharides comprise Hexose, D-Glucopyranose, D-Galactofuranose, D-Galactopyranose, L-Galactopyranose, D-Mannopyranose, D-Allopyranose, L-Altropyranose, D-Gulopyranose, L-Idopyranose, D-Talopyranose, D-Ribofuranose, D-Ribopyranose, D-Arabinofuranose, D-Arabinopyranose, L-Arabinofuranose, L-Arabinopyranose, D-Xylopyranose, D-Lyxopyranose, D-Erythrofuranose, D-Threofuranose, Heptose, L-glycero-D-manno-Heptopyranose (LDmanHep), D-glycero-D-manno-Heptopyranose (DDmanHep), 6-Deoxy-L-altropyranose, 6-Deoxy-D-gulopyranose, 6-Deoxy-D-talopyranose, 6-Deoxy-D-galactopyranose, 6-Deoxy-L-galactopyranose, 6-Deoxy-D-mannopyranose, 6-Deoxy-L-mannopyranose, 6-Deoxy-D-glucopyranose, 2-Deoxy-D-arabino-hexose, 2-Deoxy-D-erythro-pentose, 2,6-Dideoxy-D-arabino-hexopyranose, 3,6-Dideoxy-D-arabino-hexopyranose, 3,6-Dideoxy-L-arabino-hexopyranose, 3,6-Dideoxy-D-xylo-hexopyranose, 3,6-Dideoxy-D-ribo-hexopyranose, 2,6-Dideoxy-D-ribo-hexopyranose, 3,6-Dideoxy-L-xylo-hexopyranose, 2-Amino-2-deoxy-D-glucopyranose, 2-Amino-2-deoxy-D-galactopyranose, 2-Amino-2-deoxy-D-mannopyranose, 2-Amino-2-deoxy-D-allopyranose, 2-Amino-2-deoxy-L-altropyranose, 2-Amino-2-deoxy-D-gulopyranose, 2-Amino-2-deoxy-L-idopyranose, 2-Amino-2-deoxy-D-talopyranose, 2-Acetamido-2-deoxy-D-glucopyranose, 2-Acetamido-2-deoxy-D-galactopyranose, 2-Acetamido-2-deoxy-D-mannopyranose, 2-Acetamido-2-deoxy-D-allopyranose, 2-Acetamido-2-deoxy-L-altropyranose, 2-Acetamido-2-deoxy-D-gulopyranose, 2-Acetamido-2-deoxy-L-idopyranose, 2-Acetamido-2-deoxy-D-talopyranose, 2-Acetamido-2,6-dideoxy-D-galactopyranose, 2-Acetamido-2,6-dideoxy-L-galactopyranose, 2-Acetamido-2,6-dideoxy-L-mannopyranose, 2-Acetamido-2,6-dideoxy-D-glucopyranose, 2-Acetamido-2,6-dideoxy-L-altropyranose, 2-Acetamido-2,6-dideoxy-D-talopyranose, D-Glucopyranuronic acid, D-Galactopyranuronic acid, D-Mannopyranuronic acid, D-Allopyranuronic acid, L-Altropyranuronic acid, D-Gulopyranuronic acid, L-Gulopyranuronic acid, L-Idopyranuronic acid, D-Talopyranuronic acid, sialic acid, 5-Amino-3,5-dideoxy-D-glycero-D-galacto-non-2-ulosonic acid, 5-Acetamido-3,5-dideoxy-D-glycero-D-galacto-non-2-ulosonic acid, 5-Glycolylamido-3,5-dideoxy-D-glycero-D-galacto-non-2-ulosonic acid, Erythritol, Arabinitol, Xylitol, Ribitol, Glucitol, Galactitol, Mannitol, D-ribo-Hex-2-ulopyranose, D-arabino-Hex-2-ulofuranose (D-fructofuranose), D-arabino-Hex-2-ulopyranose, L-xylo-Hex-2-ulopyranose, D-lyxo-Hex-2-ulopyranose, D-threo-Pent-2-ulopyranose, D-altro-Hept-2-ulopyranose, 3-C-(Hydroxymethyl)-D-erythofuranose, 2,4,6-Trideoxy-2,4-diamino-D-glucopyranose, 6-Deoxy-3-O-methyl-D-glucose, 3-O-Methyl-D-rhamnose, 2,6-Dideoxy-3-methyl-D-ribo-hexose, 2-Amino-3-O—[(R)-1-carboxyethyl]-2-deoxy-D-glucopyranose, 2-Acetamido-3-O—[(R)-carboxyethyl]-2-deoxy-D-glucopyranose, 2-Glycolylamido-3-O—[(R)-1-carboxyethyl]-2-deoxy-D-glucopyranose, 3-Deoxy-D-lyxo-hept-2-ulopyranosaric acid, 3-Deoxy-D-manno-oct-2-ulopyranosonic acid, 3-Deoxy-D-glycero-D-galacto-non-2-ulopyranosonic acid, 5,7-Diamino-3,5,7,9-tetradeoxy-L-glycero-L-manno-non-2-ulopyranosonic acid, 5,7-Diamino-3,5,7,9-tetradeoxy-L-glycero-L-altro-non-2-ulopyranosonic acid, 5,7-Diamino-3,5,7,9-tetradeoxy-D-glycero-D-galacto-non-2-ulopyranosonic acid, 5,7-Diamino-3,5,7,9-tetradeoxy-D-glycero-D-talo-non-2-ulopyranosonic acid, 2-acetamido-2,6-dideoxy-L-arabino-4-hexulose, 2-acetamido-2,6-dideoxy-L-lyxo-4-hexulose, N-acetyl-L-rhamnosamine, N-acetyl-D-fucosamine, N-acetyl-L-pneumosamine, N-acetylmuramic acid, N-acetyl-L-quinovosamine, glucose (Glc), galactose (Gal), N-acetylglucosamine (GlcNAc), glucosamine (Glen), mannose (Man), xylose (Xyl), N-acetylmannosamine (ManNAc), N-acetylneuraminic acid, N-glycolylneuraminic acid, a sialic acid, N-acetylgalactosamine (GalNAc), galactosamine (Galn), fucose (Fuc), rhamnose (Rha), glucuronic acid, gluconic acid, fructose (Fru) and polyols.

With the term polyol is meant an alcohol containing multiple hydroxyl groups. For example, glycerol, sorbitol, or mannitol.

The term “disaccharide comprising two different monosaccharide subunits” as used herein refers to a saccharide composed of two different monosaccharide units wherein one of the monosaccharides is GlcNAc or wherein one or both monosaccharides are derived from GlcNAc. Examples of disaccharides as used herein comprises but are not limited to lacto-N-biose (Gal-b1,3-GlcNAc, LNB), galacto-N-biose (Gal-b1,3-GalNAc, Gal-b1,6-GalNAc), N-acetyllactosamine (Gal-b1,4-GlcNAc, LacNAc), LacDiNAc (GalNAc-b1,4-GlcNAc), N-acetylgalactosaminylglucose (GalNAc-b1,4-Glc), N-acetylglucosaminylglucose (GlcNAc-b1,4-Glc), Fuc-a1,3-GlcNAc, Man-b1,4-GlcNAc or ManNAc-b1,4-GlcNAc.

“Oligosaccharide” as the term is used herein and as generally understood in the state of the art, refers to a saccharide polymer containing a small number, typically three to twenty, preferably three to fifteen, more preferably three to thirteen, even more preferably three to twelve, even more preferably three to eleven, most preferably three to ten, of simple sugars, i.e., monosaccharides. The oligosaccharide as used in this disclosure can be a linear structure or can include branches. The linkage (e.g., glycosidic linkage, galactosidic linkage, glucosidic linkage, etc.) between two sugar units can be expressed, for example, as 1,4, 1->4, or (1-4), used interchangeably herein. For example, the terms “Gal-b1,4-Glc,” “Gal-b1,4-Glc,” “b-Gal-(1->4)-Glc,” “b-Gal-(1->4)-Glc,” “Galbeta1-4-Glc,” “Gal-b(1-4)-Glc” and “Gal-b(1-4)-Glc” have the same meaning, i.e., a beta-glycosidic bond links carbon-1 of galactose (Gal) with the carbon-4 of glucose (Glc). Each monosaccharide can be in the cyclic form (e.g., pyranose or furanose form). Linkages between the individual monosaccharide units may include alpha 1->2, alpha 1->3, alpha 1->4, alpha 1->6, alpha 2->1, alpha 2->3, alpha 2->4, alpha 2->6, beta 1->2, beta 1->3, beta 1->4, beta 1->6, beta 2->1, beta 2->3, beta 2->4, and beta 2->6. An oligosaccharide can contain both alpha- and beta-glycosidic bonds or can contain only beta-glycosidic bonds. The terms “glycan” and “polysaccharide” are used interchangeably and refer to a compound comprising a large number of monosaccharides linked glycosidically. The term glycan is commonly used for those compounds containing more than ten monosaccharide residues.

“Oligosaccharide” as used herein are composed of three or more monosaccharides wherein at least one of the composing monosaccharides is different from the other composing monosaccharides and wherein at least one of the monosaccharides is GlcNAc or is derived from UDP-GlcNAc. Preferably, the oligosaccharide as described herein contains monosaccharides selected from the list as used herein above. Examples of oligosaccharides of this disclosure include but are not limited to Lewis-type antigen oligosaccharides, mammalian milk oligosaccharides (MMOs) that contain GlcNAc and/or monosaccharides that are derived from UDP-GlcNAc, O-antigen, enterobacterial common antigen (ECA), the oligosaccharide repeats present in capsular polysaccharides, peptidoglycan (PG) and antigens of the human ABO blood group system.

As used herein, “mammalian milk oligosaccharide” refers to oligosaccharides that contain GlcNAc and/or monosaccharides that are derived from UDP-GlcNAc. Examples include but are not limited to 6′-sialyllactose, 3′-sialyllactose, 3,6-disialyllactose, 6,6′-disialyllactose, 8,3-disialyllactose, 3,6-disialyllacto-N-tetraose, lactodifucotetraose, lacto-N-tetraose, lacto-N-neotetraose, lacto-N-fucopentaose II, lacto-N-fucopentaose I, lacto-N-fucopentaose III, lacto-N-fucopentaose V, lacto-N-fucopentaose VI, sialyllacto-N-neotetraose d, sialyllacto-N-neotetraose c, sialyllacto-N-tetraose b, sialyllacto-N-tetraose a, lacto-N-difucohexaose I, lacto-N-difucohexaose II, lacto-N-hexaose, lacto-N-neohexaose, para-lacto-N-hexaose, monofucosylmonosialyllacto-N-neotetraose c, monofucosyl para-lacto-N-hexaose, monofucosyllacto-N-hexaose III, isomeric fucosylated lacto-N-hexaose III, isomeric fucosylated lacto-N-hexaose I, sialyllacto-N-hexaose, sialyllacto-N-neohexaose II, difucosyl-para-lacto-N-hexaose, difucosyllacto-N-hexaose, difucosyllacto-N-hexaose a, difucosyllacto-N-hexaose c.

Mammalian milk oligosaccharides or MMOs comprise oligosaccharides present in milk found in any phase during lactation including colostrum milk from humans (i.e., human milk oligosaccharides or HMOs) and mammals including but not limited to cows (Bos taurus), sheep (Ovis aries), goats (Capra aegagrus hircus), bactrian camels (Camelus bactrianus), horses (Equus ferus caballus), pigs (Sus scropha), dogs (Canis lupus familiaris), ezo brown bears (Ursus arctos yesoensis), polar bear (Ursus maritimus), Japanese black bears (Ursus thibetanus japonicus), striped skunks (Mephitis mephitis), hooded seals (Cystophora cristata), Asian elephants (Elephas maximus), African elephant (Loxodonta africana), giant anteater (Myrmecophaga tridactyla), common bottlenose dolphins (Tursiops truncates), northern minke whales (Balaenoptera acutorostrata), tammar wallabies (Macropus eugenii), red kangaroos (Macropus rufus), common brushtail possum (Trichosurus vulpecula), koalas (Phascolarctos cinereus), eastern quolls (Dasyurus viverrinus), platypus (Ornithorhynchus anatinus). Human milk oligosaccharides are also known as human identical milk oligosaccharides that are chemically identical to the human milk oligosaccharides found in human breast milk, but that are biotechnologically produced (e.g., using cell free systems or cells and organisms comprising a bacterium, a fungus, a yeast, a plant, animal, or protozoan cell, preferably metabolically engineered cells and organisms). Human identical milk oligosaccharides are marketed under the name HiMO.

As used herein, the term “0-antigen” refers to the repetitive glycan component of the surface lipopolysaccharide (LPS) of Gram-negative bacteria. The term “enterobacterial common antigen” or “ECA” refers to a specific carbohydrate antigen built of repeating units of three amino sugars, i.e., N-acetylglucosamine, N-acetyl-d-mannosaminuronic acid and 4-acetamido-4,6-dideoxy-d-galactose, which is shared by all members of the Enterobacteriaceae, and which is located in the outer leaflet of the outer membrane and in the periplasm. The term “capsular polysaccharides” refers to long-chain polysaccharides with repeat-unit structures that are present in bacterial capsules, the latter being a polysaccharide layer that lies outside the cell envelope. The terms “peptidoglycan” or “murein” refers to an essential structural element in the cell wall of most bacteria, being composed of sugars and amino acids, wherein the sugar components comprise alternating residues of beta-1,4 linked GlcNAc and N-acetylmuramic acid. As used herein, an antigen of the human ABO blood group system is an oligosaccharide. Such antigens of the human ABO blood group system are not restricted to human structures. The structures involve the A determinant GalNAc-alpha1,3(Fuc-alpha1,2)-Gal-, the B determinant Gal-alpha1,3(Fuc-alpha1,2)-Gal- and the H determinant Fuc-alpha1,2-Gal-that are present on disaccharide core structures comprising Gal-beta1,3-GlcNAc, Gal-beta1,4-GlcNAc, Gal-beta1,3-GalNAc and Gal-beta1,4-Glc.

The terms “LNT II,” “LNT-II,” “LN3,” “lacto-N-triose II,” “lacto-N-triose II,” “lacto-N-triose,” “lacto-N-triose” or “GlcNAcβ1-3Galβ1-4Glc” as used in this disclosure, are used interchangeably. The terms “LNT,” “lacto-N-tetraose,” “lacto-N-tetraose” or “Galβ1-3GlcNAcβ1-3Galβ1-4Glc” as used in this disclosure, are used interchangeably. The terms “LNnT,” “lacto-N-neotetraose,” “lacto-N-neotetraose,” “neo-LNT” or “Galβ1-4GlcNAcβ1-3Galβ1-4Glc” as used in this disclosure, are used interchangeably.

The terms “3-sialyllactose,” “3′-sialyllactose,” “alpha-2,3-sialyllactose,” “alpha 2,3 sialyllactose,” “α-2,3-sialyllactose,” “α 2,3 sialyllactose,” “Neu5Aca2-3Galβ1-4Glc,” “3SL,” “3′SL,” “3-SL” or “3′-SL” as used in this disclosure, are used interchangeably. The terms “6-sialyllactose,” “6′-sialyllactose,” “alpha-2,6-sialyllactose,” “alpha 2,6 sialyllactose,” “α-2,6-sialyllactose,” “α 2,6 sialyllactose,” “Neu5Aca2-6Galβ1-4Glc,” “6SL,” “6′ SL,” “6-SL” or “6′-SL” as used in this disclosure, are used interchangeably.

The terms “LSTa,” “LS-Tetrasaccharide a,” “Sialyl-lacto-N-tetraose a,” “sialyllacto-N-tetraose a” or “Neu5Ac-a2,3-Gal-b1,3-GlcNAc-b1,3-Gal-b1,4-Glc” as used in this disclosure, are used interchangeably. The terms “LSTb,” “LS-Tetrasaccharide b,” “Sialyl-lacto-N-tetraose b,” “sialyllacto-N-tetraose b” are used interchangeably and refer to “Gal-b1,3-(Neu5Ac-a2,6)-GlcNAc-b1,3-Gal-b1,4-Glc.”

The terms “LSTc,” “LS-Tetrasaccharide c,” “Sialyl-lacto-N-tetraose c,” “sialyllacto-N-tetraose c,” “sialyllacto-N-neotetraose c” or “Neu5Ac-a2,6-Gal-b1,4-GlcNAc-b1,3-Gal-b1,4-Glc” as used in this disclosure, are used interchangeably. The terms “LSTd,” “LS-Tetrasaccharide d,” “Sialyl-lacto-N-tetraose d,” “sialyllacto-N-tetraose d,” “sialyllacto-N-neotetraose d” or “Neu5Ac-a2,3-Gal-b1,4-GlcNAc-b1,3-Gal-b1,4-Glc.” The terms “DSLNnT” and “Disialyllacto-N-neotetraose” are used interchangeably and refer to Neu5Ac-a2,6-Gal-b1,4-GlcNAc-b1,3-[Neu5Ac-a2,6]-Gal-b1,4-Glc. The terms “DSLNT,” “DS-LNT” and “Disialyllacto-N-tetraose” are used interchangeably and refer to Neu5Ac-a2,3-Gal-b1,3-[Neu5Ac-a2,6]-GlcNAc-b1,3-Gal-b1,4-Glc.

The term “purified” refers to material that is substantially or essentially free from components that interfere with the activity of the biological molecule. For cells, saccharides, nucleic acids, and polypeptides, the term “purified” refers to material that is substantially or essentially free from components that normally accompany the material as found in its native state. Typically, purified saccharides, oligosaccharides, proteins or nucleic acids of the disclosure are at least about 50%, 55%, 60%, 65%, 70%, 75%, 80% or 85% pure, usually at least about 90%, 91%, 92%, 93%, 94%, 95%, 96%, 97%, 98%, or 99% pure as measured by band intensity on a silver-stained gel or other method for determining purity. Purity or homogeneity can be indicated by a number of means well known in the art, such as polyacrylamide gel electrophoresis of a protein or nucleic acid sample, followed by visualization upon staining. For certain purposes high resolution will be needed and HPLC or a similar means for purification utilized. For oligosaccharides, purity can be determined using methods such as but not limited to thin layer chromatography, gas chromatography, NMR, HPLC, capillary electrophoresis or mass spectroscopy.

The term “cultivation” refers to the culture medium wherein the cell is cultivated or fermented, the cell itself, and the di- and/or oligosaccharides that are produced by the cell in whole broth, i.e., inside (intracellularly) as well as outside (extracellularly) of the cell. As used herein, the term “cell productivity index (CPI)” refers to the mass of the product produced by the cells divided by the mass of the cells produced in the culture.

The terms “excretion,” “excreted,” “excretes” as used herein refer to the transfer of molecules from inside the cell to out of the cell by any method, e.g., through active or passive transport, through the use of the endoplasmic reticulum (ER) or any vesicles derived thereof.

The term “cell excreting a di- or oligosaccharide out of the cell” as used herein refers to a cell that synthesizes a di- or oligosaccharide in the cytosol and transfers the di- or oligosaccharide to the outside of the cell by any method, e.g., through active or passive transport.

The term “precursor” as used herein refers to substances that are taken up or synthetized by the cell for the specific production of a di- or oligosaccharide according to this disclosure. In this sense a precursor can be an acceptor as defined herein, but can also be another substance, metabolite, a mono-, di- or oligosaccharide, which is first modified within the cell as part of the biochemical synthesis route of the di- and/or oligosaccharide of this disclosure. Examples of such precursors comprise the acceptors as defined herein, and/or glucose, galactose, fructose, glycerol, sialic acid, N-acetylneuraminic acid, fucose, mannose, maltose, sucrose, lactose, dihydroxyacetone, glucosamine, N-acetylglucosamine, mannosamine, N-acetylmannosamine, galactosamine, N-acetylgalactosamine, galactosyllactose, N-acetyllactosamine (LacNAc), lacto-N-biose (LNB), phosphorylated sugars like e.g., but not limited to glucose-1-phosphate, galactose-1-phosphate, glucose-6-phosphate, fructose-6-phosphate, fructose-1,6-bisphosphate, glycerol-3-phosphate, glyceraldehyde-3-phosphate, dihydroxyacetone-phosphate, glucosamine-6-phosphate, N-acetyl-glucosamine-6-phosphate, N-acetylmannosamine-6-phosphate, N-acetyl-Neuraminic acid-9-phosphate, N-acetylglucosamine-1-phosphate, mannose-6-phosphate, mannose-1-phosphate and nucleotide-activated sugars as defined herein like e.g., UDP-glucose, UDP-galactose, UDP-N-acetylglucosamine, CMP-sialic acid, GDP-mannose, GDP-4-dehydro-6-deoxy-α-D-mannose, and/or GDP-fucose.

Optionally, the cell is transformed to comprise at least one nucleic acid sequence encoding a protein selected from the group comprising lactose transporter, N-acetylneuraminic acid transporter, fucose transporter, transporter for a nucleotide-activated sugar wherein the transporter internalizes a to the medium added precursor for di- and/or oligosaccharide synthesis.

The term “acceptor” as used herein refers to di- or oligosaccharides that can be modified by a glycosyltransferase. Examples of such acceptors comprise lactose, lacto-N-biose (LNB), lacto-N-triose, lacto-N-tetraose (LNT), lacto-N-neotetraose (LNnT), N-acetyllactosamine (LacNAc), lacto-N-pentaose (LNP), lacto-N-neopentaose, para lacto-N-pentaose, para lacto-N-neopentaose, lacto-N-novopentaose I, lacto-N-hexaose (LNH), lacto-N-neohexaose (LNnH), para lacto-N-neohexaose (pLNnH), para lacto-N-hexaose (pLNH), lacto-N-heptaose, lacto-N-neoheptaose, para lacto-N-neoheptaose, para lacto-N-heptaose, lacto-N-octaose (LNO), lacto-N-neooctaose, iso lacto-N-octaose, para lacto-N-octaose, iso lacto-N-neooctaose, novo lacto-N-neooctaose, para lacto-N-neooctaose, iso lacto-N-nonaose, novo lacto-N-nonaose, lacto-N-nonaose, lacto-N-decaose, iso lacto-N-decaose, novo lacto-N-decaose, lacto-N-neodecaose, galactosyllactose, a lactose extended with 1, 2, 3, 4, 5, or a multiple of N-acetyllactosamine units and/or 1, 2, 3, 4, 5, or a multiple of, Lacto-N-biose units, and oligosaccharide containing 1 or multiple N-acetyllactosamine units and/or 1 or multiple lacto-N-biose units or an intermediate into oligosaccharide, fucosylated and sialylated versions thereof.

### DETAILED DESCRIPTION

According to a first embodiment, this disclosure provides a metabolically engineered cell for the production of a glycosylated product that is derived from UDP-GlcNAc and that comprises a di- or oligosaccharide that is composed of at least two different monosaccharide subunits. Herein, a metabolically engineered cell is provided that is capable to express, preferably expresses, a variant yeast or fungal glutamine-fructose-6-phosphate aminotransferase, is capable to synthesize, preferably synthesizes, UDP-GlcNAc and is capable to express, preferably expresses, a glycosyltransferase wherein the UDP-GlcNAc is used by the cell to produce the glycosylated product.

According to a second embodiment, this disclosure provides a method for the production of a glycosylated product derived from UDP-GlcNAc and comprising a di- or oligosaccharide that is composed of at least two different monosaccharide subunits. The method comprises the steps of:
(a) providing a cell that
   (i) is capable to express, preferably expresses, a variant yeast or fungal glutamine-fructose-6-phosphate aminotransferase, and
   (ii) is capable to synthesize, preferably synthesizes, UDP-N-acetylglucosamine (UDP-GlcNAc), and
   (iii) is capable to express, preferably expresses, a glycosyltransferase, and
   (iv) uses the UDP-GlcNAc to produce the glycosylated product, and
(b) cultivating the cell under conditions permissive to produce the glycosylated product,
(c) preferably, separating the glycosylated product from the cultivation.

According to the disclosure, the method for the production of a glycosylated product derived from UDP-GlcNAc and comprising a di- or oligosaccharide that is composed of at least two different monosaccharide subunits makes use of a metabolically engineered cell as disclosed herein.

In the scope of this disclosure, permissive conditions are understood to be conditions relating to physical or chemical parameters including but not limited to temperature, pH, pressure, osmotic pressure and product/precursor/acceptor concentration.

In a particular embodiment, the permissive conditions may include a temperature-range of 30+/−20 degrees centigrade, a pH-range of 2.0-10.0.

In another preferred embodiment of the method of the disclosure, the cultivation is fed with a precursor for the synthesis of the glycosylated product. In a further preferred embodiment of the method, the cultivation is fed with at least two precursors for the synthesis of the glycosylated product.

According to one aspect of the method and/or cell of the disclosure, the cell synthesizes UDP-GlcNAc and uses the UDP-GlcNAc in a pathway to synthesize a glycosylated product comprising a UDP-GlcNAc derived di- or oligosaccharide that is composed of at least two different monosaccharide subunits. In a preferred embodiment of the method and/or cell, the cell synthesizes UDP-GlcNAc and uses the UDP-GlcNAc in a pathway to synthesize a UDP-GlcNAc derived di- or oligosaccharide that is composed of at least two different monosaccharide subunits. As used herein, the cell can use different pathways to synthesize a UDP-GlcNAc derived di- or oligosaccharide that is composed of at least two different monosaccharide subunits; and/or a glycosylated product comprising a UDP-GlcNAc derived di- or oligosaccharide that is composed of at least two different monosaccharide subunits.

In a preferred embodiment of the method and/or cell of the disclosure, the cell uses the synthesized UDP-GlcNAc to transfer the GlcNAc moiety from the UDP-GlcNAc by a specific glycosyltransferase expressed in the cell onto a monosaccharide acceptor as defined herein but not being GlcNAc to synthesize a UDP-GlcNAc derived disaccharide that is composed of two different monosaccharide subunits.

In another preferred embodiment of the method and/or cell of the disclosure, the cell uses the synthesized UDP-GlcNAc to transfer the GlcNAc moiety from the UDP-GlcNAc by a specific glycosyltransferase onto a disaccharide acceptor as defined herein but not being GlcNAc-GlcNAc to synthesize a UDP-GlcNAc derived oligosaccharide that is composed of at least two different monosaccharide subunits.

In another preferred embodiment of the method and/or cell of the disclosure, the cell uses the synthesized UDP-GlcNAc and one or more other nucleotide-activated sugar(s) that is/are not derived from UDP-GlcNAc to transfer the GlcNAc moiety and the monosaccharide building block(s) from the UDP-GlcNAc and the one or more other nucleotide-activated sugar(s), respectively, by specific glycosyltransferases, respectively, onto a saccharide acceptor as defined herein to synthesize a UDP-GlcNAc derived oligosaccharide that is composed of at least two different monosaccharide subunits.

In another preferred embodiment of the method and/or cell of the disclosure, the cell uses the synthesized UDP-GlcNAc to produce a UDP-GlcNAc derived nucleoside(s) and transfers the monosaccharide from the UDP-GlcNAc derived nucleoside by a specific glycosyltransferase onto a monosaccharide acceptor as defined herein to synthesize a UDP-GlcNAc derived disaccharide that is composed of two different monosaccharide subunits.

In another preferred embodiment of the method and/or cell of the disclosure, the cell uses the synthesized UDP-GlcNAc to produce one or more UDP-GlcNAc derived nucleoside(s) and transfers one or more monosaccharides from the one or more UDP-GlcNAc derived nucleoside(s) and/or GlcNAc from the synthesized UDP-GlcNAc by specific glycosyltransferases, respectively, onto a saccharide acceptor as defined herein to synthesize a UDP-GlcNAc derived oligosaccharide that is composed of at least two different monosaccharide subunits.

In another preferred embodiment of the method and/or cell of the disclosure, the cell uses the synthesized UDP-GlcNAc to produce one or more UDP-GlcNAc derived nucleoside(s) and transfers one or more monosaccharides from the one or more UDP-GlcNAc derived nucleoside(s), one or more monosaccharides from one or more nucleosides that are not derived from UDP-GlcNAc and/or GlcNAc from the UDP-GlcNAc by specific glycosyltransferases, respectively, onto a saccharide acceptor as defined herein to synthesize a UDP-GlcNAc derived oligosaccharide that is composed of at least two different monosaccharide subunits.

In another preferred embodiment of the method and/or cell of the disclosure, the cell uses the synthesized UDP-GlcNAc and one or more other nucleotide-activated sugar(s) that is/are not derived from UDP-GlcNAc to transfer the GlcNAc moiety and the monosaccharide building block(s) from the UDP-GlcNAc and the one or more other nucleotide-activated sugar(s), respectively, by specific glycosyltransferases, respectively, onto an acceptor as defined herein to synthesize a glycosylated product comprising a UDP-GlcNAc derived oligosaccharide that is composed of at least two different monosaccharide subunits.

In another preferred embodiment of the method and/or cell of the disclosure, the cell uses the synthesized UDP-GlcNAc to produce one or more UDP-GlcNAc derived nucleoside(s) and transfers one or more monosaccharides from the one or more UDP-GlcNAc derived nucleoside(s) and/or GlcNAc from the synthesized UDP-GlcNAc by specific glycosyltransferases, respectively, onto an acceptor as defined herein to synthesize a glycosylated product comprising a UDP-GlcNAc derived oligosaccharide that is composed of at least two different monosaccharide subunits.

In another preferred embodiment of the method and/or cell of the disclosure, the cell uses the synthesized UDP-GlcNAc to produce one or more UDP-GlcNAc derived nucleoside(s) and transfers one or more monosaccharides from the one or more UDP-GlcNAc derived nucleoside(s), one or more monosaccharides from one or more nucleosides that are not derived from UDP-GlcNAc and/or GlcNAc from the UDP-GlcNAc by specific glycosyltransferases, respectively, onto an acceptor as defined herein to synthesize a glycosylated product comprising a UDP-GlcNAc derived oligosaccharide that is composed of at least two different monosaccharide subunits.

In a preferred embodiment of the method and/or cell of the disclosure, the nucleosides that are not derived from UDP-GlcNAc are added to the cultivation as precursor(s). In a more preferred embodiment of the method and/or cell of the disclosure, the nucleosides that are not derived from UDP-GlcNAc are synthesized by the cell of the disclosure.

In another preferred embodiment of the method and/or cell of the disclosure, the acceptor for synthesis of the UDP-GlcNAc derived di- or oligosaccharide is added to the cultivation as precursor. In a more preferred embodiment of the method and/or cell of the disclosure, the acceptor is synthesized by the cell of the disclosure.

This disclosure provides different types of cells for the production of a glycosylated product derived from UDP-GlcNAc and comprising a di- or oligosaccharide that is composed of at least two different monosaccharide subunits with a metabolically engineered cell. For example, this disclosure provides a cell wherein the cell expresses one variant yeast or fungal glutamine-fructose-6-phosphate aminotransferase and the cell synthesizes UDP-GlcNAc that is used by the cell to produce the glycosylated product. This disclosure also provides a cell wherein the cell expresses one variant yeast or fungal glutamine-fructose-6-phosphate aminotransferase and the cell synthesizes one UDP-GlcNAc-derived nucleoside that is used by the cell to produce the glycosylated product. This disclosure also provides a cell wherein the cell expresses one variant yeast or fungal glutamine-fructose-6-phosphate aminotransferase and the cell synthesizes UDP-GlcNAc and/or one or more UDP-GlcNAc-derived nucleoside(s) that is/are used by the cell to produce the glycosylated product. This disclosure also provides a cell wherein the cell expresses two variant yeast or fungal glutamine-fructose-6-phosphate aminotransferases and the cell synthesizes UDP-GlcNAc that is used by the cell to produce the glycosylated product. This disclosure also provides a cell wherein the cell expresses two variant yeast or fungal glutamine-fructose-6-phosphate aminotransferases and the cell synthesizes one UDP-GlcNAc-derived nucleoside that is used by the cell to produce the glycosylated product. This disclosure also provides a cell wherein the cell expresses two variant yeast or fungal glutamine-fructose-6-phosphate aminotransferases and the cell synthesizes UDP-GlcNAc and/or one or more UDP-GlcNAc-derived nucleoside(s) that is/are used by the cell to produce the glycosylated product. This disclosure also provides a cell wherein the cell expresses three or more variant yeast or fungal glutamine-fructose-6-phosphate aminotransferases and the cell synthesizes UDP-GlcNAc and/or one or more UDP-GlcNAc-derived nucleoside(s) that is/are used by the cell to produce the glycosylated product.

According to a preferred embodiment of the method and/or cell according to the disclosure, the metabolically engineered cell is modified with at least one gene expression module comprising the variant yeast or fungal glutamine-fructose-6-phosphate aminotransferase wherein the expression from the expression module is constitutive or is conditional upon non-chemical induction or repression.

The expression modules are also known as transcriptional units and comprise polynucleotides for expression of recombinant genes including coding gene sequences and appropriate transcriptional and/or translational control signals that are operably linked to the coding genes. The control signals comprise promoter sequences, untranslated regions, ribosome binding sites, terminator sequences. The expression modules can contain elements for expression of one single recombinant gene but can also contain elements for expression of more recombinant genes or can be organized in an operon structure for integrated expression of two or more recombinant genes. The polynucleotides may be produced by recombinant DNA technology using techniques well-known in the art. Methods that are well known to those skilled in the art to construct expression modules include, for example, in vitro recombinant DNA techniques, synthetic techniques, and in vivo genetic recombination. See, for example, the techniques described in Sambrook et al. (2001) Molecular Cloning: a laboratory manual, 3rd Edition, Cold Spring Harbor Laboratory Press, CSH, New York or to Current Protocols in Molecular Biology, John Wiley and Sons, N.Y. (1989 and yearly updates).

According to a preferred aspect of this disclosure, the cell is modified with one or more expression modules comprising the variant yeast or fungal glutamine-fructose-6-phosphate aminotransferase. As used herein, the cell can be modified with one expression module for the variant yeast or fungal glutamine-fructose-6-phosphate aminotransferase. The cell can also be modified with one expression module for two or more of the variant yeast or fungal glutamine-fructose-6-phosphate aminotransferases. The cell can also be modified with one expression module for the variant yeast or fungal glutamine-fructose-6-phosphate aminotransferase and one or more expression modules for one or more other recombinant genes that are distinct from the variant yeast or fungal glutamine-fructose-6-phosphate aminotransferase. The cell can also be modified with two or more expression modules wherein at least one of the expression modules contains elements for expression of one or more of the variant yeast or fungal glutamine-fructose-6-phosphate aminotransferase(s).

The other recombinant genes can be involved in the expression of a polypeptide acting in the synthesis of the glycosylated product; or the recombinant genes can be linked to other pathways in the metabolically engineered cell that are not involved in the synthesis of the glycosylated product. The recombinant genes encode endogenous proteins with a modified expression or activity, preferably the endogenous proteins are overexpressed; or the recombinant genes encode heterologous proteins that are heterogeneously introduced and expressed in the modified cell, preferably overexpressed. The endogenous proteins can have a modified expression in the cell that also expresses a heterologous protein.

The expression modules can be integrated in the genome of the cell or can be presented to the cell on a vector. The vector can be present in the form of a plasmid, cosmid, phage, liposome, or virus, which is to be stably transformed/transfected into the metabolically engineered cell. Such vectors include, among others, chromosomal, episomal and virus-derived vectors, e.g., vectors derived from bacterial plasmids, from bacteriophage, from transposons, from yeast episomes, from insertion elements, from yeast chromosomal elements, from viruses, and vectors derived from combinations thereof, such as those derived from plasmid and bacteriophage genetic elements, such as cosmids and phagemids. These vectors may contain selection markers such as but not limited to antibiotic markers, auxotrophic markers, toxin-antitoxin markers, RNA sense/antisense markers. The expression system constructs may contain control regions that regulate as well as engender expression. Generally, any system or vector suitable to maintain, propagate or express polynucleotides and/or to express a polypeptide in a host may be used for expression in this regard. The appropriate DNA sequence may be inserted into the expression system by any of a variety of well-known and routine techniques, such as, for example, those set forth in Sambrook et al., see above. For recombinant production, cells can be genetically engineered to incorporate expression systems or portions thereof or polynucleotides of the disclosure. Introduction of a polynucleotide into the cell can be affected by methods described in many standard laboratory manuals, such as Davis et al., Basic Methods in Molecular Biology, (1986), and Sambrook et al., 1989, supra.

According to a more preferred aspect of this disclosure, the expression from each of the expression modules present in the metabolically engineered cells is constitutive or conditional upon non-chemical induction or repression.

As used herein, constitutive expression should be understood as expression of a gene that is transcribed continuously in an organism. Examples of constitutive promoters that are often used in recombinant host cells comprise bacterial promoters like e.g., the spc ribosomal protein operon promoter P_{spc}, the b-lactamase promoter P_{bla}, the P_{cat} promoter, the P1 and P2 promoters of the rrnB ribosomal RNA operon, the promoters of ompC and of yfgF; yeast promoters like e.g., ACT1, CCW12, CYC1, FBA1, HXT7-391, GPD, MFa1, PAB1, PDC1, PGK1, PYK1, TDH3, TEF1 or TP11; or other promoters like e.g., the P_{L} promoter of phage lambda (Da Silva and Srikrishnan, FEMS Yeast Res. (2012) 12: 197-214; Redden et al., FEMS Yeast Res. (2015) 15: 1-10; Shimada et al. 2014, PLOS ONE 9(6): e100908) or promoter sequences originating from libraries like e.g., described by Redden and Alper (Nat. Commun. 2015, 6, 7810), Liu et al. (Microb. Cell Fact. 2020, 19, 38), Xu et al. (Microb. Cell Fact. 2021, 20, 148) and Lee et al. (ACS Synth. Biol. 2015, 4(9), 975-986).

As used herein, expression that is conditional upon non-chemical induction or repression should be understood as a facultative or regulatory expression of a gene that is only expressed upon a certain natural condition of the host (e.g., mating phase of budding yeast, stationary phase of bacteria, organism being in labor, or during lactation), as a response to an environmental change (e.g., anaerobic or aerobic growth, oxidative stress, temperature changes like e.g., heat-shock or cold-shock, osmolarity, light conditions) or dependent on the position of the developmental stage or the cell cycle of the host cell including but not limited to apoptosis and autophagy. Examples of promoters that give conditional expression upon non-chemical induction or repression comprise oxygen-responsive promoters (like e.g., DAN1 from S. cerevisiae, nar from E. coli, bacterial globin promoters), oxidative stress-responsive promoters (like e.g., CTT1, Skn7, TRX2 or Yap1 from yeasts or oxyR, soxR, soxS, sodA or ahpC from bacteria), heat-shock responsive promoters (like e.g., CPR6, HSP26, HSP82, HSP104, SSA1, SSA3, SSA4 or YDJ1 from yeasts; or ahpF, DnaK, GroEL or HtpG from bacteria) and promoters active in stationary phase (like e.g., the E. coli osmY promoter) (Farr and Kogoma, Microbiol. Rev. 1991, 55(4): 561-585; Imlay J. A., Annu. Rev. Microbiol. 2015, 69: 93-108; Lara et al., J. Biol. Eng. 2017, 11:39; Lee et al., Biotechnol. Bioeng. 2003, 82(3): 271-277; Loprosert et al., Mol. Microbiol. 2000, 37: 1504-1514; Morano et al., Genetics 2012, 190(4): 1157-1195).

As used herein, expression that is conditional upon chemical induction or repression should be understood as a facultative or regulatory expression of a gene that is only expressed upon presence or absence of a certain chemical compound comprising carbon sources (like e.g., glucose, allo-lactose, lactose, galactose, glycerol, arabinose), alcohols (like e.g., methanol, ethanol), acidic compounds (like e.g., acetate, formate), IPTG, metal ions (like e.g., aluminum, copper, zinc, nitrogen), phosphates, aromates (like e.g., xylene). Examples of promoters that give conditional expression upon chemical induction or repression comprise the rhamnose-inducible rhaBAD promoter from E. coli, the arabinose-inducible pBAD promoter of the E. coli araBAD operon, the IPTG-inducible lac promoter from E. coli or the IPTG/lactose-inducible T7 promoter or the E. coli salt (NaCl)-inducible promoter proU (Marschall et al. 2016, Appl. Microbiol. Biotechnol. 100, 5719-5728) or the ADH1, GAL1-GAL 10, MET3, MET25, P_{CUP1} and PHO5 promoters from yeast. The 1500 bp promoter P_{ADH1} is activated during growth on glucose and is downregulated following glucose depletion and during ethanol consumption. A short variant of the promoter, P_{ADH1s} with a deletion of 1100 bp in the upstream sequence, shifts expression to the early ethanol growth phase with activity increasing into the late ethanol consumption phase. Restoring of 300 bp of the upstream fragment resulted in a middle ADH1 promoter (pADH1m) that is activated in early exponential growth and maintains activity into the late ethanol consumption phase (Ruohonen et al., 1995, J. Biotechnol. 39: 193-203). The ADH2 promoter is tightly regulated by glucose repression. In the presence of glucose, GAL promoters are completely off, and in the presence of galactose, a 1000-fold increase in expression can be achieved in just 4 hours. The promoter P_{CUP1} can induce 20-fold expression in the presence of Cu²⁺. The PHO5 promoter is regulated by inorganic phosphate whereas the MET3 and MET25 promoters are repressed by methionine or S-adenosylmethionine (Keren et al., Mol. Sys. Biol. (2013) 9:701; Redden et al., FEMS Yeast Res. (2015) 15: 1-10).

It should be understood that the lists of promoter sequences as provided herein are given by way of illustration and are not intended to be limited.

According to one aspect of the method and/or cell of the disclosure, the cell expresses a variant yeast or fungal glutamine-fructose-6-phosphate aminotransferase wherein the variant is a protein that has glutamine-fructose-6-phosphate aminotransferase activity and that comprises a polypeptide sequence according to any one of SEQ ID NOs: 39, 40, 41, 42, 43, 44, 45, 46, 47, 48, 49, 50, 51, 52 or 53 that differs from SEQ ID NO: 01 by a V12L, a Q96H, a Q157R and/or an E343V mutation, or that is a functional homolog, variant or derivative of any one of SEQ ID NOs: 01, 02, 03, 04, 05, 06, 07, 08, 09, 10, 11, 12, 13, 14, 15, 16, 17, 18, 19, 20, 21, 22, 23, 24, 25, 26, 27, 28, 29, 30, 31, 32, 33, 34, 35, 36, 37 or 38 having at least 80% overall sequence identity to the full-length of any one of the polypeptides with SEQ ID NOs: 01, 02, 03, 04, 05, 06, 07, 08, 09, 10, 11, 12, 13, 14, 15, 16, 17, 18, 19, 20, 21, 22, 23, 24, 25, 26, 27, 28, 29, 30, 31, 32, 33, 34, 35, 36, 37 or 38, or that is a functional homolog, variant or derivative of any one of SEQ ID NOs: 01, 02, 03, 04, 05, 06, 07, 08, 09, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52 or 53 having at least 80% overall sequence identity to the full-length of any one of the polypeptides with SEQ ID NOs: 01, 02, 03, 04, 05, 06, 07, 08, 09, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52 or 53 and differing from SEQ ID NO: 01 by a V12L, a Q96H, a Q157R and/or an E343V mutation.

As used herein, a protein having an amino acid sequence having at least 80% sequence identity to the full-length sequence of any of the enlisted proteins, is to be understood as that the sequence has 80%, 81%, 82%, 83%, 84%, 85%, 86%, 87%, 88%, 89%, 90%, 91%, 91.50%, 92.00%, 92.50%, 93.00%, 93.50%, 94.00%, 94.50%, 95.00%, 95,50%, 96.00%, 96,50%, 97.00%, 97,50%, 98.00%, 98,50%, 99.00%, 99,50%, 99,60%, 99,70%, 99,80%, 99,90% sequence identity to the full-length of the amino acid sequence of the respective protein.

At least 80% overall sequence identity to the full-length of any one of the polypeptides with SEQ ID NOs: 01, 02, 03, 04, 05, 06, 07, 08, 09, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52 or 53 is to be understood as at least 80%, 81%, 82%, 83%, 84%, 85%, 86%, 87%, 88%, 89%, 90%, 91%, 91.50%, 92.00%, 92.50%, 93.00%, 93.50%, 94.00%, 94.50%, 95.00%, 95,50%, 96.00%, 96,50%, 97.00%, 97,50%, 98.00%, 98,50%, 99.00%, 99,50%, 99,60%, 99,70%, 99,80%, 99,90% overall sequence identity to any one of the polypeptides with SEQ ID NOs: 01, 02, 03, 04, 05, 06, 07, 08, 09, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52 or 53, respectively, as given herein.

According to a preferred aspect of the method and/or cell of the disclosure, the cell expresses a variant yeast or fungal glutamine-fructose-6-phosphate aminotransferase wherein the variant is a protein that has glutamine-fructose-6-phosphate aminotransferase activity and that comprises a polypeptide sequence according to any one of SEQ ID NOs: 39, 40, 41, 42, 43, 44, 45, 46, 47, 48, 49, 50, 51, 52 or 53 that differs from SEQ ID NO: 01 by a V12L, a Q96H, a Q157R and/or an E343V mutation.

According to another preferred aspect of the method and/or cell of the disclosure, the cell expresses a variant yeast or fungal glutamine-fructose-6-phosphate aminotransferase wherein the variant is a protein that has glutamine-fructose-6-phosphate aminotransferase activity and that is a polypeptide comprising or consisting of an amino acid sequence that is at least 80.0% sequence identical over a stretch of at least 200 amino acid residues to the amino acid sequence of any one of SEQ ID NOs: 01, 02, 03, 04, 05, 06, 07, 08, 09, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52 or 53, respectively, and that differs from SEQ ID NO: 01 by a V12L, a Q96H, a Q157R and/or an E343V mutation. Preferably, the variant comprises or consists of an amino acid sequence that is at least 80.0%, at least 85.0%, at least 90.0%, at least 95.0%, at least 96.0%, at least 97.0%, at least 98.0%, at least 98.5%, or at least 99% identical to the amino acid sequence over a stretch of at least 200, at least 210, at least 220, at least 230, at least 240, at least 250, preferably up to the total number of amino acid residues to the amino acid sequence of any one of SEQ ID NOs: 01, 02, 03, 04, 05, 06, 07, 08, 09, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52 or 53, respectively, and that differs from SEQ ID NO: 01 by a V12L, a Q96H, a Q157R and/or an E343V mutation.

According to another preferred aspect of the method and/or cell of the disclosure, the expression or activity of any one of the variant yeast or fungal glutamine-fructose-6-phosphate aminotransferases is modified in the cell. According to a more preferred aspect of the method and/or cell of the disclosure, modified expression should be understood as defined herein. According to another more preferred embodiment of the method and/or cell of the disclosure, modified activity of an enzyme should be understood as enhanced, increased and/or improved activity of an enzyme comprising e.g., a better conversion rate, a faster reaction rate, reduced sensitivity toward feedback inhibition, improved kinetics and higher substrate affinity compared to the native activity of the enzyme.

As used herein, the variant yeast or fungal glutamine-fructose-6-phosphate aminotransferase is a protein that has glutamine-fructose-6-phosphate aminotransferase activity and is capable to convert D-fructose-6-phosphate+L-glutamine into D-glucosamine-6-phosphate+L-glutamate. D-fructose-6-phosphate (or fructose-6-phosphate) belongs to the class of organic compounds known as hexose phosphates and is described at PubChem with identifier PubChem CID 69507 (National Center for Biotechnology Information (2020). PubChem Compound Summary for CID 69507, Fructose-6-phosphate, with entry created on 16 Sep. 2004 and Modified on 10 Oct. 2020 on pubchem.ncbi.nlm.nih.gov/compound/Fructose-6-phosphate. Retrieved Oct. 16, 2020) with reference to the Human Metabolome Database version 4.0 (Wishart et al., Nucleic Acids Res. 2007, January; 35(Database issue):D521-526) with identifier HMDB0000124 as created on 16 Nov. 2015, Updated on 9 Oct. 2020 and retrieved from /www.hmdb.ca/metabolites/HMDB0000124 on Oct. 16 2020). Hexose phosphates are carbohydrate derivatives containing a hexose substituted by one or more phosphate groups. Fructose 6-phosphate exists in prokaryotes and in all eukaryotes, ranging from yeast to humans. Fructose 6-phosphate participates in a number of enzymatic reactions. Fructose 6-phosphate can be biosynthesized from glucosamine 6-phosphate, which is catalyzed by the enzyme glucosamine-6-phosphate isomerase 1. Fructose 6-phosphate can be converted into glucose 6-phosphate through the action of the enzyme glucose-6-phosphate isomerase. Reversibly, fructose 6-phosphate can be biosynthesized from Beta-D-glucose 6-phosphate that is mediated by glucose-6-phosphate isomerase. Fructose 6-phosphate is involved in e.g., the pentose phosphate pathway, the gluconeogenesis pathway, and the glycolysis pathway.

L-glutamine is not only one of the 20 standard amino acids but is also an important intermediate in the synthesis of a variety of nitrogen-containing compounds. The most important enzymes related to the glutamine metabolism of host cells are glutamine synthetase and glutaminase. Glutamine synthetase catalyzes the conversion of glutamate to glutamine using ammonia as nitrogen source (glutamate+NH4⁺+ATP giving rise to glutamine+ADP+Pi). Glutaminase is an enzyme that catalyzes the hydrolysis of glutamine to glutamate and an ammonium ion.

According to another aspect of this disclosure, a method and a metabolically engineered cell are provided wherein the cell synthesizes UDP-N-acetylglucosamine (UDP-GlcNAc). As used herein, the cell synthesizes UDP-GlcNAc and the UDP-GlcNAc could be further converted into a UDP-GlcNAc-derived nucleoside. According to this disclosure, the cell synthesizes UDP-GlcNAc by a multistep conversion of D-glucosamine-6-phosphate that is made available in the cell by action of the variant yeast or fungal glutamine-fructose-6-phosphate aminotransferase. Different multistep conversions could be used by the host cell to convert the D-glucosamine-6-phosphate into UDP-GlcNAc. For example, a first set of conversion reactions comprise a first conversion of D-glucosamine-6-phosphate into N-acetyl-D-glucosamine-6-phosphate by action of a glucosamine 6-phosphate N-acetyltransferase like e.g., GNA1 from S. cerevisiae, followed by a second reaction wherein N-acetyl-D-glucosamine-6-phosphate is converted into N-acetyl-D-glucosamine-1-phosphate by action of a phosphoacetylglucosamine mutase like e.g., PCM1 from S. cerevisiae, followed by a final conversion of N-acetyl-D-glucosamine-1-phosphate into UDP-GlcNAc by action of a UDP-N-acetylglucosamine pyrophosphorylase like e.g., QRI1 from S. cerevisiae. Another multistep conversion of D-glucosamine-6-phosphate into UDP-GlcNAc involves a first conversion of D-glucosamine-6-phosphate into D-glucosamine-1-phosphate by action of a phosphoglucosamine mutase like e.g., glmM from E. coli and a subsequent conversion of D-glucosamine-1-phosphate into UDP-GlcNAc by action of a UDP-N-acetylglucosamine pyrophosphorylase and a glucosamine-1-phosphate N-acetyltransferase or by action of a bifunctional enzyme like e.g., glmU of E. coli having both UDP-N-acetylglucosamine pyrophosphorylase and glucosamine-1-phosphate N-acetyltransferase activity.

In a preferred aspect of this disclosure, the cell synthesizes one or more nucleosides derived from UDP-GlcNAc. For example, the UDP-GlcNAc-derived nucleoside UDP-N-acetylgalactosamine (UDP-GalNAc) can be synthesized from UDP-GlcNAc by the action of a single-step reaction using a UDP-N-acetylglucosamine 4-epimerase like e.g., wbgU from Plesiomonas shigelloides, gne from Yersinia enterocolitica or wbpP from Pseudomonas aeruginosa serotype 06. The UDP-GlcNAc-derived nucleoside CMP-Neu5Ac can be synthesized from UDP-GlcNAc by a well-ordered multistep reaction involving an N-acetylglucosamine-6-phosphate 2-epimerase or UDP-GlcNAc 2′-epimerase converting UDP-GlcNAc into UDP and ManNAc (like e.g., neuC from C. jejuni or Acinetobacter baumannii), an N-acetylneuraminate synthase subsequently converting ManNAc into Neu5Ac (sialic acid) whilst using phosphoenolpyruvate (PEP) (like e.g., neuB from E. coli, C. jejuni or N. meningitidis) and an N-acylneuraminate cytidylyltransferase or CMP-sialic acid synthetase finally synthesizing CMP-Neu5Ac from Neu5Ac and CTP (like e.g., neuA from C. jejuni or N. meningitidis). CMP-Neu5Ac can also be synthesized from UDP-GlcNAc via an alternative route involving a bifunctional UDP-GlcNAc 2′-epimerase/ManNAc kinase converting UDP-GlcNAc into UDP and ManNAc and subsequent phosphorylation of ManNAc into ManNAc-6-phosphate (like e.g., gne from M. musculus), a Neu5Ac-9-phosphate synthase converting ManNAc-6-phosphate whilst using PEP into Neu5Ac-9-phosphate (like e.g., Neu5Ac-9-phosphate synthase from R. norvegicus), a Neu5Ac-9-phosphate phosphatase dephosphorylating Neu5Ac-9-phosphate into Neu5Ac (like e.g., Neu5Ac-9-Pase from R. norvegicus) and a CMP-sialic acid synthetase finally synthesizing CMP-Neu5Ac from Neu5Ac and CTP (like e.g., Cmas from M. musculus). On its turn, CMP-Neu5Ac can be converted into CMP-Neu5Gc via a hydroxylation reaction performed by a vertebrate CMP-Neu5Ac hydroxylase (CMAH) enzyme. UDP-ManNAc can be synthesized directly from UDP-GlcNAc via an epimerization reaction performed by a UDP-GlcNAc 2-epimerase (like e.g., cap5P from Staphylococcus aureus, RffE from E. coli, Cps19fK from S. pneumoniae, and RfbC from S. enterica). UDP-D-N-acetylglucosamine is also the common precursor for the production of 2-acetamido-2,6-dideoxy-L-hexoses in the gluco-(quinovosamine), galacto-, manno- (rhamnosamine) and talo-configurations. For example, UDP-GlcNAc can be converted into UDP-2-acetamido-2,6-dideoxy-L-lyxo-4-hexulose by a multifunctional enzyme like WbvB from Vibrio cholerae serotype 037. This UDP-2-acetamido-2,6-dideoxy-L-lyxo-4-hexulose can be converted by a C-4 reductase like WbvR from V. cholerae serotype 037 to yield UDP-2-acetamido-L-rhamnose. This UDP-2-acetamido-L-rhamnose can be epimerized to UDP-2-acetamido-L-quinovose by WbvD, also an enzyme from V. cholerae serotype 037. A parallel pathway for the synthesis of UDP-N-acetyl-D-fucosamine (UDP-FucNAc) from UDP-GlcNAc can be performed using the three enzymes WbjB, WbjC, and WbjD from Pseudomonas aeruginosa O11 or CapE, CapF, and CapG from Staphylococcus aureus type 5. UPD-GlcNAc can also be converted to UDP-2-acetamido-2,6-dideoxy-b-L-arabino-4-hexulose using “inverting” 4,6-dehydratases like e.g., PseB from H. pylori or FlaA1 from P. aeruginosa. WbjC from P. aeruginosa 011 and CapF from S. aureus type 5 can also be used to convert UDP-2-acetamido-2,6-dideoxy-b-L-arabino-4-hexulose to UDP-N-acetyl-L-pneumosamine (UDP-L-PneNAc).

In another preferred aspect of this disclosure, the cell further synthesizes a nucleotide-sugar or nucleoside chosen from the list comprising UDP-N-acetylgalactosamine (UDP-GalNAc), UDP-N-acetylmannosamine (UDP-ManNAc), UDP-glucose (UDP-Glc), UDP-galactose (UDP-Gal), GDP-mannose (GDP-Man), UDP-glucuronate, UDP-galacturonate, UDP-2-acetamido-2,6-dideoxy-L-arabino-4-hexulose, UDP-2-acetamido-2,6-dideoxy-L-lyxo-4-hexulose, UDP-N-acetyl-L-rhamnosamine (UDP-L-RhaNAc or UDP-2-acetamido-2,6-dideoxy-L-mannose), dTDP-N-acetylfucosamine, UDP-N-acetylfucosamine (UDP-L-FucNAc or UDP-2-acetamido-2,6-dideoxy-L-galactose), UDP-N-acetyl-L-pneumosamine (UDP-L-PneNAC or UDP-2-acetamido-2,6-dideoxy-L-talose), UDP-N-acetylmuramic acid, UDP-N-acetyl-L-quinovosamine (UDP-L-QuiNAc or UDP-2-acetamido-2,6-dideoxy-L-glucose), CMP-sialic acid (CMP-Neu5Ac), CMP-N-glycolylneuraminic acid (CMP-Neu5Gc), GDP-fucose (GDP-Fuc), GDP-rhamnose and UDP-xylose.

According to another aspect of this disclosure, the cell uses the UDP-N-acetylglucosamine (UDP-GlcNAc) in the production of a glycosylated product comprising a di- or oligosaccharide that is composed of at least two different monosaccharide subunits. In a preferred aspect of this disclosure, at least one of the monosaccharide subunits is chosen from the list comprising N-acetylglucosamine, N-acetylmannosamine, N-acetylgalactosamine, 2-acetamido-2,6-dideoxy-L-arabino-4-hexulose, 2-acetamido-2,6-dideoxy-L-lyxo-4-hexulose, N-acetyl-L-rhamnosamine, N-acetyl-D-fucosamine, N-acetyl-L-pneumosamine, N-acetylmuramic acid, N-acetyl-L-quinovosamine, N-acetylneuraminic acid, and N-glycolylneuraminic acid.

In an aspect of this disclosure, the disaccharide comprises glycan structures composed of two different monosaccharide subunits wherein at least one of the monosaccharide subunits is chosen from the list comprising N-acetylglucosamine, N-acetylmannosamine, N-acetylgalactosamine, 2-acetamido-2,6-dideoxy-L-arabino-4-hexulose, 2-acetamido-2,6-dideoxy-L-lyxo-4-hexulose, N-acetyl-L-rhamnosamine, N-acetyl-D-fucosamine, N-acetyl-L-pneumosamine, N-acetylmuramic acid, N-acetyl-L-quinovosamine, N-acetylneuraminic acid, and N-glycolylneuraminic acid. As used herein, both monosaccharide subunits from the disaccharide can be chosen from the list comprising N-acetylglucosamine, N-acetylmannosamine, N-acetylgalactosamine, 2-acetamido-2,6-dideoxy-L-arabino-4-hexulose, 2-acetamido-2,6-dideoxy-L-lyxo-4-hexulose, N-acetyl-L-rhamnosamine, N-acetyl-D-fucosamine, N-acetyl-L-pneumosamine, N-acetylmuramic acid, N-acetyl-L-quinovosamine, N-acetylneuraminic acid, and N-glycolylneuraminic acid but should be different from each other.

Alternatively, a first monosaccharide subunit is chosen from the list comprising N-acetylglucosamine, N-acetylmannosamine, N-acetylgalactosamine, 2-acetamido-2,6-dideoxy-L-arabino-4-hexulose, 2-acetamido-2,6-dideoxy-L-lyxo-4-hexulose, N-acetyl-L-rhamnosamine, N-acetyl-D-fucosamine, N-acetyl-L-pneumosamine, N-acetylmuramic acid, N-acetyl-L-quinovosamine, N-acetylneuraminic acid, and N-glycolylneuraminic acid and a second monosaccharide subunit that is different from the first monosaccharide subunit is chosen from the list comprising N-acetylglucosamine, N-acetylmannosamine, N-acetylgalactosamine, 2-acetamido-2,6-dideoxy-L-arabino-4-hexulose, 2-acetamido-2,6-dideoxy-L-lyxo-4-hexulose, N-acetyl-L-rhamnosamine, N-acetyl-D-fucosamine, N-acetyl-L-pneumosamine, N-acetylmuramic acid, N-acetyl-L-quinovosamine, N-acetylneuraminic acid, N-glycolylneuraminic acid, glucose, galactose, mannose, fucose, rhamnose, xylose, glucuronate and galacturonate.

Examples of the disaccharides comprise lacto-N-biose (Gal-b1,3-GlcNAc, LNB), galacto-N-biose (Gal-b1,3-GalNAc, Gal-b1,6-GalNAc), N-acetyllactosamine (Gal-b1,4-GlcNAc, LacNAc), LacDiNAc (GalNAc-b1,4-GlcNAc), N-acetylgalactosaminylglucose (GalNAc-b1,4-Glc), N-acetylglucosaminylglucose (GlcNAc-b1,4-Glc), Fuc-a1,3-GlcNAc, Man-b1,4-GlcNAc or ManNAc-b1,4-GlcNAc.

In another aspect of this disclosure, the oligosaccharide comprises glycan structures composed of three or more monosaccharide subunits wherein at least one of the monosaccharide subunits is different from the other monosaccharide subunits of the oligosaccharide and wherein at least one of the monosaccharide subunits of the oligosaccharide is chosen from the list comprising N-acetylglucosamine, N-acetylmannosamine, N-acetylgalactosamine, 2-acetamido-2,6-dideoxy-L-arabino-4-hexulose, 2-acetamido-2,6-dideoxy-L-lyxo-4-hexulose, N-acetyl-L-rhamnosamine, N-acetyl-D-fucosamine, N-acetyl-L-pneumosamine, N-acetylmuramic acid, N-acetyl-L-quinovosamine, N-acetylneuraminic acid, and N-glycolylneuraminic acid. As used herein, the oligosaccharide can be composed of three monosaccharide subunits wherein one of the monosaccharides is chosen from the list comprising N-acetylglucosamine, N-acetylmannosamine, N-acetylgalactosamine, 2-acetamido-2,6-dideoxy-L-arabino-4-hexulose, 2-acetamido-2,6-dideoxy-L-lyxo-4-hexulose, N-acetyl-L-rhamnosamine, N-acetyl-D-fucosamine, N-acetyl-L-pneumosamine, N-acetylmuramic acid, N-acetyl-L-quinovosamine, N-acetylneuraminic acid, and N-glycolylneuraminic acid and wherein the first monosaccharide is different from the other two monosaccharide subunits of the oligosaccharide wherein the latter two monosaccharides are chosen from the list comprising N-acetylglucosamine, N-acetylmannosamine, N-acetylgalactosamine, 2-acetamido-2,6-dideoxy-L-arabino-4-hexulose, 2-acetamido-2,6-dideoxy-L-lyxo-4-hexulose, N-acetyl-L-rhamnosamine, N-acetyl-D-fucosamine, N-acetyl-L-pneumosamine, N-acetylmuramic acid, N-acetyl-L-quinovosamine, N-acetylneuraminic acid, N-glycolylneuraminic acid, glucose, galactose, mannose, fucose, rhamnose, xylose, glucuronate and galacturonate. As used herein, the oligosaccharide comprises glycan structures composed of three or more monosaccharide subunits, wherein at least one of the monosaccharides is different from the other subunits present in the oligosaccharide and wherein one of the monosaccharide subunits is chosen from the list comprising N-acetylglucosamine, N-acetylmannosamine, N-acetylgalactosamine, 2-acetamido-2,6-dideoxy-L-arabino-4-hexulose, 2-acetamido-2,6-dideoxy-L-lyxo-4-hexulose, N-acetyl-L-rhamnosamine, N-acetyl-D-fucosamine, N-acetyl-L-pneumosamine, N-acetylmuramic acid, N-acetyl-L-quinovosamine, N-acetylneuraminic acid, N-glycolylneuraminic acid.

Examples of the oligosaccharides comprise 6′-sialyllactose, 3′-sialyllactose, 3,6-disialyllactose, 6,6′-disialyllactose, 3,6-disialyllacto-N-tetraose, 8,3-disialyllactose, lacto-N-triose, lacto-N-tetraose, lacto-N-neotetraose, sialyllacto-N-neotetraose d, sialyllacto-N-neotetraose c, sialyllacto-N-tetraose b, sialyllacto-N-tetraose a, lacto-N-difucohexaose I, lacto-N-difucohexaose II, lacto-N-hexaose, lacto-N-neohexaose, para-lacto-N-hexaose, monofucosylmonosialyllacto-N-neotetraose c, monofucosyl para-lacto-N-hexaose, monofucosyllacto-N-hexaose III, isomeric fucosylated lacto-N-hexaose III, isomeric fucosylated lacto-N-hexaose I, sialyllacto-N-hexaose, sialyllacto-N-neohexaose II, difucosyl-para-lacto-N-hexaose, difucosyllacto-N-hexaose, difucosyllacto-N-hexaose a, difucosyllacto-N-hexaose c or galactosylated chitosan.

In another preferred aspect of this disclosure, the glycosylated product is chosen from the list comprising a mammalian milk di- or oligosaccharide that contains GlcNAc and/or monosaccharides that are derived from UDP-GlcNAc, preferably a human milk di- or oligosaccharide that contains GlcNAc and/or monosaccharides that are derived from UDP-GlcNAc, O-antigen, enterobacterial common antigen (ECA), the oligosaccharide repeats present in capsular polysaccharides, peptidoglycan (PG) and antigens of the human ABO blood group system.

In another aspect of the method and/or cell of the disclosure, the cell expresses a glycosyltransferase that is chosen from the list comprising fucosyltransferases, sialyltransferases, galactosyltransferases, glucosyltransferases, mannosyltransferases, N-acetylglucosaminyltransferases, N-acetylgalactosaminyltransferases, N-acetylmannosaminyltransferases, xylosyltransferases, glucuronyltransferases, galacturonyltransferases, glucosaminyltransferases, N-glycolylneuraminyltransferases, rhamnosyltransferases, N-acetylrhamnosyltransferases, UDP-4-amino-4,6-dideoxy-N-acetyl-beta-L-altrosamine transaminases and fucosaminyltransferases as defined herein.

In a preferred aspect of the method and/or cell of the disclosure, the fucosyltransferase is chosen from the list comprising alpha-1,2-fucosyltransferase, alpha-1,3-fucosyltransferase, alpha-1,3/4-fucosyltransferase, alpha-1,4-fucosyltransferase and alpha-1,6-fucosyltransferase. In another preferred aspect of the method and/or cell of the disclosure, the sialyltransferase is chosen from the list comprising alpha-2,3-sialyltransferase, alpha-2,6-sialyltransferase and alpha-2,8-sialyltransferase. In another preferred aspect of the method and/or cell of the disclosure, the galactosyltransferase is chosen from the list comprising beta-1,3-galactosyltransferase, N-acetylglucosamine beta-1,3-galactosyltransferase, beta-1,4-galactosyltransferase, N-acetylglucosamine beta-1,4-galactosyltransferase, alpha-1,3-galactosyltransferase and alpha-1,4-galactosyltransferase. In another preferred aspect of the method and/or cell of the disclosure, the glucosyltransferase is chosen from the list comprising alpha-glucosyltransferase, beta-1,2-glucosyltransferase, beta-1,3-glucosyltransferase and beta-1,4-glucosyltransferase. In another preferred aspect of the method and/or cell of the disclosure, the mannosyltransferase is chosen from the list comprising alpha-1,2-mannosyltransferase, alpha-1,3-mannosyltransferase and alpha-1,6-mannosyltransferase. In another preferred aspect of the method and/or cell of the disclosure, the N-acetylglucosaminyltransferase is chosen from the list comprising galactoside beta-1,3-N-acetylglucosaminyltransferase and beta-1,6-N-acetylglucosaminyltransferase. In another preferred aspect of the method and/or cell of the disclosure, the N-acetylgalactosaminyltransferase is an alpha-1,3-N-acetylgalactosaminyltransferase.

In another aspect of the method and/or cell of the disclosure, the cell further expresses any one or more of the enzymes chosen from the list comprising glucosamine 6-phosphate N-acetyltransferase, phosphoacetylglucosamine mutase, UDP-N-acetylglucosamine pyrophosphorylase, galactoside beta-1,3-N-acetylglucosaminyltransferase, UTP-glucose-1-phosphate uridylyltransferase, UDP-glucose 4-epimerase, N-acetylglucosamine beta-1,3-galactosyltransferase, N-acetylglucosamine beta-1,4-galactosyltransferase, lactose permease, UDP-N-acetylglucosamine 2-epimerase, N-acetylneuraminate synthase, N-acylneuraminate cytidylyltransferase, glucose-6-phosphate isomerase or UDP-2-acetamido-2,6-dideoxy-L-talose 2-epimerase as defined herein.

In a further aspect of the method and/or cell of the disclosure, the cell is modified in the expression or activity of at least one of the enzymes and/or glycosyltransferases. In a preferred embodiment, the enzyme and/or glycosyltransferase is an endogenous protein of the cell with a modified expression or activity, preferably the endogenous enzyme and/or glycosyltransferase is overexpressed; alternatively the enzyme and/or glycosyltransferase is a heterologous protein that is heterogeneously introduced and expressed in the cell, preferably overexpressed. The endogenous enzyme and/or glycosyltransferase can have a modified expression in the cell that also expresses a heterologous enzyme and/or glycosyltransferase.

In another preferred embodiment of the method and/or cell of the disclosure, during the cultivation the cell excretes the glycosylated product out of the cell. In a more preferred embodiment of the method and/or cell of the disclosure, during the cultivation the cell excretes a di- or oligosaccharide out of the cell. As used herein, the excretion of the glycosylated product comprising a di- or oligosaccharide is done by any method, e.g., through active or passive transport, through the use of the endoplasmic reticulum (ER) or any vesicles derived thereof.

According to another embodiment, this disclosure provides a yeast or fungal cell that excretes a di- or oligosaccharide out of the cell. In a preferred embodiment, this disclosure provides a yeast or fungal cell that excretes a di- or oligosaccharide comprising at least one monosaccharide unit chosen from the list comprising N-acetylglucosamine, N-acetylmannosamine, N-acetylgalactosamine, 2-acetamido-2,6-dideoxy-L-arabino-4-hexulose, 2-acetamido-2,6-dideoxy-L-lyxo-4-hexulose, N-acetyl-L-rhamnosamine, N-acetyl-D-fucosamine, N-acetyl-L-pneumosamine, N-acetylmuramic acid, N-acetyl-L-quinovosamine, N-acetylneuraminic acid, or N-glycolylneuraminic acid. In a more preferred embodiment, the yeast or fungal cell that excretes a di- or oligosaccharide comprising at least one monosaccharide unit chosen from the list comprising N-acetylglucosamine, N-acetylmannosamine, N-acetylgalactosamine, 2-acetamido-2,6-dideoxy-L-arabino-4-hexulose, 2-acetamido-2,6-dideoxy-L-lyxo-4-hexulose, N-acetyl-L-rhamnosamine, N-acetyl-D-fucosamine, N-acetyl-L-pneumosamine, N-acetylmuramic acid, N-acetyl-L-quinovosamine, N-acetylneuraminic acid, or N-glycolylneuraminic acid is a metabolically engineered cell as described herein.

In an additional embodiment, this disclosure provides a method for the excretion a di- or oligosaccharide by a cell. In a preferred additional embodiment, the method makes use of a metabolically engineered cell as described herein. In a more preferred additional embodiment, the method can be used for excretion of a di- or oligosaccharide comprising at least one monosaccharide unit chosen from the list comprising N-acetylglucosamine, N-acetylmannosamine, N-acetylgalactosamine, 2-acetamido-2,6-dideoxy-L-arabino-4-hexulose, 2-acetamido-2,6-dideoxy-L-lyxo-4-hexulose, N-acetyl-L-rhamnosamine, N-acetyl-D-fucosamine, N-acetyl-L-pneumosamine, N-acetylmuramic acid, N-acetyl-L-quinovosamine, N-acetylneuraminic acid, or N-glycolylneuraminic acid.

In another embodiment of the method and/or cell as described herein, the cell is using at least one precursor for the synthesis of the glycosylated product. In a preferred embodiment, the cell is using two or more precursors for the synthesis of the glycosylated product.

In another embodiment of the method and/or cell as described herein, the cell is producing a precursor for the synthesis of the glycosylated product. In a preferred embodiment, the cell is producing one or more precursors for the synthesis of the glycosylated product. In a more preferred embodiment, the cell is modified for optimized production of any one of the precursors for the synthesis of the glycosylated product.

In a preferred embodiment, this disclosure provides a method for the production of a glycosylated product with a cell wherein the cell completely converts any one of the precursors into the glycosylated product.

The term “precursor” should be understood as explained in the definitions as disclosed herein.

Another aspect of the disclosure provides for a method and a cell wherein a glycosylated product derived from UDP-GlcNAc and comprising a di- or oligosaccharide that is composed of at least two different monosaccharide subunits is produced in and/or by a fungal, yeast, bacterial, insect, animal or plant expression system or cell, or protozoan cell as described herein. The expression system or cell is chosen from the list comprising a bacterium, a yeast, or a fungus, or refers to a plant, animal or protozoan cell. The latter bacterium preferably belongs to the phylum of the Proteobacteria or the phylum of the Firmicutes or the phylum of the Cyanobacteria or the phylum Deinococcus-Thermus. The latter bacterium belonging to the phylum Proteobacteria belongs preferably to the family Enterobacteriaceae, preferably to the species Escherichia coli. The latter bacterium preferably relates to any strain belonging to the species Escherichia coli such as but not limited to Escherichia coli B, Escherichia coli C, Escherichia coli W, Escherichia coli K12, Escherichia coli Nissle. More specifically, the latter term relates to cultivated Escherichia coli strains—designated as E. coli K12 strains—that are well-adapted to the laboratory environment, and, unlike wild type strains, have lost their ability to thrive in the intestine. Well-known examples of the E. coli K12 strains are K12 Wild type, W3110, MG1655, M182, MC1000, MC1060, MC1061, MC4100, JM101, NZN111 and AA200. Hence, this disclosure specifically relates to a mutated and/or transformed Escherichia coli cell or strain as indicated above wherein the E. coli strain is a K12 strain. More preferably, the Escherichia coli K12 strain is E. coli MG1655. The latter bacterium belonging to the phylum Firmicutes belongs preferably to the Bacilli, preferably Lactobacilliales, with members such as Lactobacillus lactis, Leuconostoc mesenteroides, or Bacillales with members such as from the genus Bacillus, such as Bacillus subtilis or, B. amyloliquefaciens. The latter Bacterium belonging to the phylum Actinobacteria, preferably belonging to the family of the Corynebacteriaceae, with members Corynebacterium glutamicum or C. afermentans, or belonging to the family of the Streptomycetaceae with members Streptomyces griseus or S. fradiae. The latter yeast preferably belongs to the phylum of the Ascomycota or the phylum of the Basidiomycota or the phylum of the Deuteromycota or the phylum of the Zygomycetes. The latter yeast belongs preferably to the genus Saccharomyces (with members like e.g., Saccharomyces cerevisiae, S. bayanus, S. boulardii), Zygosaccharomyces, Pichia (with members like e.g., Pichia pastoris, P. anomala, P. kluyveri), Komagataella, Hansenula, Kluyveromyces (with members like e.g., Kluyveromyces lactis, K marxianus, K thermotolerans) or Debaromyces. The latter fungus belongs preferably to the genus Rhizopus, Dictyostelium, Penicillium, Mucor or Aspergillus. Plant cells include cells of flowering and non-flowering plants, as well as algal cells, for example, Chlamydomonas, Chlorella, etc. Preferably, the plant is a tobacco, alfalfa, rice, tomato, cotton, rapeseed, soy, maize, or corn plant. The latter animal cell is preferably derived from non-human mammals (e.g., cattle, buffalo, pig, sheep, mouse, rat), birds (e.g., chicken, duck, ostrich, turkey, pheasant), fish (e.g., swordfish, salmon, tuna, sea bass, trout, catfish), invertebrates (e.g., lobster, crab, shrimp, clams, oyster, mussel, sea urchin), reptiles (e.g., snake, alligator, turtle), amphibians (e.g., frogs) or insects (e.g., fly, nematode) or is a genetically modified cell line derived from human cells excluding embryonic stem cells. Both human and non-human mammalian cells are preferably chosen from the list comprising an epithelial cell like e.g., a mammary epithelial cell, an embryonic kidney cell (e.g., HEK293 or HEK 293T cell), a fibroblast cell, a COS cell, a Chinese hamster ovary (CHO) cell, a murine myeloma cell like e.g., an N20, SP2/0 or YB2/0 cell, an NIH-3T3 cell, a non-mammary adult stem cell or derivatives thereof such as described in WO21067641. The latter insect cell is preferably derived from Spodoptera frugiperda like e.g., Sf9 or Sf21 cells, Bombyx mori, Mamestra brassicae, Trichoplusia ni like e.g., BTI-TN-5B1-4 cells or Drosophila melanogaster like e.g., Drosophila S2 cells. The latter protozoan cell preferably is a Leishmania tarentolae cell.

The microorganism or cell as used herein is capable to grow on a monosaccharide, disaccharide, oligosaccharide, polysaccharide, polyol, a complex medium including molasses, corn steep liquor, peptone, tryptone, yeast extract or a mixture thereof like e.g., a mixed feedstock, preferably a mixed monosaccharide feedstock like e.g., hydrolyzed sucrose as the main carbon source. With the term main is meant the most important carbon source for the bioproducts of interest, biomass formation, carbon dioxide and/or by-products formation (such as acids and/or alcohols, such as acetate, lactate, and/or ethanol), i.e., 20, 30, 40, 50, 60, 70, 75, 80, 85, 90, 95, 98, 99% of all the required carbon is derived from the above-indicated carbon source. In one embodiment of the disclosure, the carbon source is the sole carbon source for the organism, i.e., 100% of all the required carbon is derived from the above-indicated carbon source. Common main carbon sources comprise but are not limited to glucose, glycerol, fructose, maltose, lactose, arabinose, malto-oligosaccharides, maltotriose, sorbitol, xylose, rhamnose, sucrose, galactose, mannose, methanol, ethanol, trehalose, starch, cellulose, hemi-cellulose, molasses, corn-steep liquor, high-fructose syrup, acetate, citrate, lactate and pyruvate. With the term complex medium is meant a medium for which the exact constitution is not determined. Examples are molasses, corn steep liquor, peptone, tryptone or yeast extract.

In a further preferred embodiment, the microorganism or cell described herein is using a split metabolism having a production pathway and a biomass pathway as described in WO 2012/007481, which is herein incorporated by reference. The organism can, for example, be genetically modified to accumulate fructose-6-phosphate by altering the genes selected from the phosphoglucoisomerase gene, phosphofructokinase gene, fructose-6-phosphate aldolase gene, fructose isomerase gene, and/or fructose:PEP phosphotransferase gene.

According to this disclosure, the method as described herein preferably comprises a step of separating the glycosylated product from the cultivation.

The term “separating from the cultivation” means harvesting, collecting, or retrieving the glycosylated product from the cell and/or the medium of its growth.

The glycosylated product can be separated in a conventional manner from the aqueous culture medium, in which the cell was grown. In case the glycosylated product is still present in the cells producing the glycosylated product, conventional manners to free or to extract the glycosylated product out of the cells can be used, such as cell destruction using high pH, heat shock, sonication, French press, homogenization, enzymatic hydrolysis, chemical hydrolysis, solvent hydrolysis, detergent, hydrolysis, . . . . The culture medium and/or cell extract together and separately can then be further used for separating the glycosylated product. This preferably involves clarifying the glycosylated product containing mixture to remove suspended particulates and contaminants, particularly cells, cell components, insoluble metabolites and debris produced by culturing the genetically modified cell. In this step, the glycosylated product containing mixture can be clarified in a conventional manner. Preferably, the glycosylated product containing mixture is clarified by centrifugation, flocculation, decantation and/or filtration. Another step of separating the glycosylated product from the glycosylated product containing mixture preferably involves removing substantially all the proteins, as well as peptides, amino acids, RNA and DNA and any endotoxins and glycolipids that could interfere with the subsequent separation step, from the glycosylated product containing mixture, preferably after it has been clarified. In this step, proteins and related impurities can be removed from the glycosylated product containing mixture in a conventional manner. Preferably, proteins, salts, by-products, color, endotoxins and other related impurities are removed from the glycosylated product containing mixture by ultrafiltration, nanofiltration, two-phase partitioning, reverse osmosis, microfiltration, activated charcoal or carbon treatment, treatment with non-ionic surfactants, enzymatic digestion, tangential flow high-performance filtration, tangential flow ultrafiltration, electrophoresis (e.g., using slab-polyacrylamide or sodium dodecyl sulphate-polyacrylamide gel electrophoresis (PAGE)), affinity chromatography (using affinity ligands including e.g., DEAE-Sepharose, poly-L-lysine and polymyxin-B, endotoxin-selective adsorber matrices), ion exchange chromatography (such as but not limited to cation exchange, anion exchange, mixed bed ion exchange, inside-out ligand attachment), hydrophobic interaction chromatography and/or gel filtration (i.e., size exclusion chromatography), particularly by chromatography, more particularly by ion exchange chromatography or hydrophobic interaction chromatography or ligand exchange chromatography or electrodialysis. With the exception of size exclusion chromatography, proteins and related impurities are retained by a chromatography medium or a selected membrane, the glycosylated product remains in the glycosylated product containing mixture.

In a further preferred embodiment, the methods as described herein also provide for a further purification of the glycosylated product. A further purification of the glycosylated product may be accomplished, for example, by use of (activated) charcoal or carbon, nanofiltration, ultrafiltration, electrophoresis, enzymatic treatment or ion exchange, temperature adjustment, pH adjustment or pH adjustment with an alkaline or acidic solution to remove any remaining DNA, protein, LPS, endotoxins, or other impurity. Alcohols, such as ethanol, and aqueous alcohol mixtures can also be used. Another purification step is accomplished by crystallization, evaporation or precipitation of the product. Another purification step is to dry, e.g., spray dry, lyophilize, spray freeze dry, freeze spray dry, band dry, belt dry, vacuum band dry, vacuum belt dry, drum dry, roller dry, vacuum drum dry or vacuum roller dry the produced glycosylated product.

In an exemplary embodiment, the separation and purification of the produced glycosylated product is made in a process, comprising the following steps in any order:
a) contacting the cultivation or a clarified version thereof with a nanofiltration membrane with a molecular weight cut-off (MWCO) of 600-3500 Da ensuring the retention of produced glycosylated product and allowing at least a part of the proteins, salts, by-products, color and other related impurities to pass,
b) conducting a diafiltration process on the retentate from step a), using the membrane, with an aqueous solution of an inorganic electrolyte, followed by optional diafiltration with pure water to remove excess of the electrolyte,
c) and collecting the retentate enriched in the glycosylated product in the form of a salt from the cation of the electrolyte.

In an alternative exemplary embodiment, the separation and purification of the produced glycosylated product is made in a process, comprising the following steps in any order: subjecting the cultivation or a clarified version thereof to two membrane filtration steps using different membranes, wherein
one membrane has a molecular weight cut-off of between about 300 to about 500 Dalton, and
the other membrane as a molecular weight cut-off of between about 600 to about 800 Dalton.

In an alternative exemplary embodiment, the separation and purification of the produced glycosylated product is made in a process, comprising treating the cultivation or a clarified version thereof with a strong cation exchange resin in H+-form in a step and with a weak anion exchange resin in free base form in another step, wherein the steps can be performed in any order.

In an alternative exemplary embodiment, the separation and purification of the produced glycosylated product is made in the following way. The cultivation comprising the produced glycosylated product, biomass, medium components and contaminants is applied to the following purification steps:
i) separation of biomass from the cultivation,
ii) cationic ion exchanger treatment for the removal of positively charged material,
iii) anionic ion exchanger treatment for the removal of negatively charged material,
iv) nanofiltration step and/or electrodialysis step,
wherein a purified solution comprising the produced glycosylated product at a purity of greater than or equal to 80 percent is provided. Optionally the purified solution is dried by any one or more drying steps chosen from the list comprising spray drying, lyophilization, spray freeze drying, freeze spray drying, band drying, belt drying, vacuum band drying, vacuum belt drying, drum drying, roller drying, vacuum drum drying and vacuum roller drying.

In an alternative exemplary embodiment, the separation and purification of the produced glycosylated product is made in a process, comprising the following steps in any order: enzymatic treatment of the cultivation; removal of the biomass from the cultivation; ultrafiltration; nanofiltration; and a column chromatography step. Preferably, such column chromatography is a single column or a multiple column. Further preferably, the column chromatography step is simulated moving bed chromatography. Such simulated moving bed chromatography preferably comprises i) at least 4 columns, wherein at least one column comprises a weak or strong cation exchange resin; and/or ii) four zones I, II, III and IV with different flow rates; and/or iii) an eluent comprising water; and/or iv) an operating temperature of 15 degrees to 60 degrees centigrade.

In a specific embodiment, this disclosure provides the produced glycosylated product that is dried to powder by any one or more drying steps chosen from the list comprising spray drying, lyophilization, spray freeze drying, freeze spray drying, band drying, belt drying, vacuum band drying, vacuum belt drying, drum drying, roller drying, vacuum drum drying and vacuum roller drying, wherein the dried powder contains <15 percent-wt. of water, preferably <10 percent-wt. of water, more preferably <7 percent-wt. of water, most preferably <5 percent-wt. of water.

In another aspect, this disclosure provides a vector comprising an isolated nucleic acid molecule encoding a variant yeast or fungal glutamine-fructose-6-phosphate aminotransferase as described herein.

In another aspect, this disclosure provides for the use of an isolated nucleic acid molecule encoding a variant yeast or fungal glutamine-fructose-6-phosphate aminotransferase as described herein for the production of a glycosylated product that is derived from UDP-GlcNAc and comprising a di- or oligosaccharide that is composed of at least two different monosaccharide subunits as described herein.

In another aspect, this disclosure provides for the use of a vector comprising an isolated nucleic acid molecule encoding a variant yeast or fungal glutamine-fructose-6-phosphate aminotransferase as described herein for the production of a glycosylated product as described herein.

In another aspect, this disclosure provides for the use of a metabolically engineered cell as described herein for the production of a glycosylated product as described herein.

For identification of the glycosylated product produced in the cell as described herein, the monomeric building blocks (e.g., the monosaccharide or glycan unit composition), the anomeric configuration of side chains, the presence and location of substituent groups, degree of polymerization/molecular weight and the linkage pattern can be identified by standard methods known in the art, such as, e.g., methylation analysis, reductive cleavage, hydrolysis, GC-MS (gas chromatography-mass spectrometry), MALDI-MS (Matrix-assisted laser desorption/ionization-mass spectrometry), ESI-MS (Electrospray ionization-mass spectrometry), HPLC (High-Performance Liquid chromatography with ultraviolet or refractive index detection), HPAEC-PAD (High-Performance Anion-Exchange chromatography with Pulsed Amperometric Detection), CE (capillary electrophoresis), IR (infrared)/Raman spectroscopy, and NMR (Nuclear magnetic resonance) spectroscopy techniques. The crystal structure can be solved using, e.g., solid-state NMR, FT-IR (Fourier transform infrared spectroscopy), and WAXS (wide-angle X-ray scattering). The degree of polymerization (DP), the DP distribution, and polydispersity can be determined by, e.g., viscosimetry and SEC (SEC-HPLC, high performance size-exclusion chromatography). To identify the monomeric components of the saccharide methods such as, e.g., acid-catalyzed hydrolysis, HPLC (high performance liquid chromatography) or GLC (gas-liquid chromatography) (after conversion to alditol acetates) may be used. To determine the glycosidic linkages, the saccharide is methylated with methyl iodide and strong base in DMSO, hydrolysis is performed, a reduction to partially methylated alditols is achieved, an acetylation to methylated alditol acetates is performed, and the analysis is carried out by GLC/MS (gas-liquid chromatography coupled with mass spectrometry). To determine the oligosaccharide sequence, a partial depolymerization is carried out using an acid or enzymes to determine the structures. To identify the anomeric configuration, the oligosaccharide is subjected to enzymatic analysis, e.g., it is contacted with an enzyme that is specific for a particular type of linkage, e.g., beta-galactosidase, or alpha-glucosidase, etc., and NMR may be used to analyze the products.

Products Comprising the Glycosylated Product

In some embodiments, a glycosylated product produced as described herein is incorporated into a food (e.g., human food or feed), dietary supplement, pharmaceutical ingredient, cosmetic ingredient or medicine. In some embodiments, the glycosylated product is mixed with one or more ingredients suitable for food, feed, dietary supplement, pharmaceutical ingredient, cosmetic ingredient or medicine.

In some embodiments, the dietary supplement comprises at least one prebiotic ingredient and/or at least one probiotic ingredient.

A “prebiotic” is a substance that promotes growth of microorganisms beneficial to the host, particularly microorganisms in the gastrointestinal tract. In some embodiments, a dietary supplement provides multiple prebiotics, including the glycosylated product being a prebiotic produced and/or purified by a process disclosed in this specification, to promote growth of one or more beneficial microorganisms. Examples of prebiotic ingredients for dietary supplements include other prebiotic molecules (such as HMOs) and plant polysaccharides (such as inulin, pectin, b-glucan and xylooligosaccharide). A “probiotic” product typically contains live microorganisms that replace or add to gastrointestinal microflora, to the benefit of the recipient. Examples of such microorganisms include Lactobacillus species (for example, L. acidophilus and L. bulgaricus), Bifidobacterium species (for example, B. animalis, B. longum and B. infantis (e.g., Bi-26)), and Saccharomyces boulardii. In some embodiments, a glycosylated product produced and/or purified by a process of this specification is orally administered in combination with such microorganism.

Examples of further ingredients for dietary supplements include disaccharides (such as lactose), monosaccharides (such as glucose and galactose), thickeners (such as gum arabic), acidity regulators (such as trisodium citrate), water, skimmed milk, and flavorings.

In some embodiments, the glycosylated product is incorporated into a human baby food (e.g., infant formula). Infant formula is generally a manufactured food for feeding to infants as a complete or partial substitute for human breast milk. In some embodiments, infant formula is sold as a powder and prepared for bottle- or cup-feeding to an infant by mixing with water. The composition of infant formula is typically designed to roughly mimic human breast milk. In some embodiments, a glycosylated product produced and/or purified by a process in this specification is included in infant formula to provide nutritional benefits similar to those provided by the oligosaccharides in human breast milk. In some embodiments, the glycosylated product is mixed with one or more ingredients of the infant formula. Examples of infant formula ingredients include non-fat milk, carbohydrate sources (e.g., lactose), protein sources (e.g., whey protein concentrate and casein), fat sources (e.g., vegetable oils—such as palm, high oleic safflower oil, rapeseed, coconut and/or sunflower oil; and fish oils), vitamins (such as vitamins A, Bb, Bi2, C and D), minerals (such as potassium citrate, calcium citrate, magnesium chloride, sodium chloride, sodium citrate and calcium phosphate) and possibly human milk oligosaccharides (HMOs). Such HMOs may include, for example, DiFL, lacto-N-triose II, LNT, LNnT, lacto-N-fucopentaose I, lacto-N-neofucopentaose, lacto-N-fucopentaose II, lacto-N-fucopentaose III, lacto-N-fucopentaose V, lacto-N-neofucopentaose V, lacto-N-difucohexaose I, lacto-N-difucohexaose II, 6′-galactosyllactose, 3′-galactosyllactose, lacto-N-hexaose and lacto-N-neohexaose.

In some embodiments, the one or more infant formula ingredients comprise non-fat milk, a carbohydrate source, a protein source, a fat source, and/or a vitamin and mineral.

In some embodiments, the one or more infant formula ingredients comprise lactose, whey protein concentrate and/or high oleic safflower oil.

In some embodiments, the glycosylated products concentration in the infant formula is approximately the same concentration as the glycosylated products concentration generally present in human breast milk.

In some embodiments, the glycosylated product is incorporated into a feed preparation, wherein the feed is chosen from the list comprising pet food, animal milk replacer, veterinary product, veterinary feed supplement, nutrition supplement, post weaning feed, or creep feed.

As will be shown in the examples herein, the methods and the cell of the disclosure preferably provide at least one of the following surprising advantages:
higher titers of the glycosylated product (g/L),
higher production rate r (g of the glycosylated product/L/h),
higher cell performance index CPI (g of the glycosylated product/g X),
higher specific productivity Qp (g of the glycosylated product/g X/h),
higher yield on sucrose Ys (g of the glycosylated product/g sucrose),
higher sucrose uptake/conversion rate Qs (g sucrose/g X/h),
higher lactose conversion/consumption rate rs (g lactose/h),
higher excretion of the glycosylated product, and/or
higher growth speed of the production host,
when compared to a method and a host for production of the glycosylated product lacking a variant yeast or fungal glutamine-fructose-6-phosphate aminotransferase as described herein.

Unless defined otherwise, all technical and scientific terms used herein generally have the same meaning as commonly understood by one of ordinary skill in the art to which this disclosure belongs. Generally, the nomenclature used herein and the laboratory procedures in cell culture, molecular genetics, organic chemistry and nucleic acid chemistry and hybridization described above and below are those well-known and commonly employed in the art. Standard techniques are used for nucleic acid and peptide synthesis. Generally, purification steps are performed according to the manufacturer's specifications.

Further advantages follow from the specific embodiments and the examples. It goes without saying that the abovementioned features and the features that are still to be explained below can be used not only in the respectively specified combinations, but also in other combinations or on their own, without departing from the scope of this disclosure.

This disclosure relates to following specific embodiments:
1. A metabolically engineered cell for production of a glycosylated product comprising a di- or oligosaccharide that is composed of at least two different monosaccharide subunits, the cell:
   (i) expressing a variant yeast or fungal glutamine-fructose-6-phosphate aminotransferase, and
   (ii) synthesizing UDP-N-acetylglucosamine (UDP-GlcNAc), and
   (iii) expressing a glycosyltransferase,
   wherein the cell uses the UDP-GlcNAc to produce the glycosylated product.
2. Cell according to embodiment 1, wherein the cell is a metabolically engineered cell modified with at least one gene expression module comprising the variant yeast or fungal glutamine-fructose-6-phosphate aminotransferase, preferably the expression from the expression module is constitutive or conditional upon non-chemical induction or repression.
3. Cell according to any one of previous embodiments, wherein the variant yeast or fungal glutamine-fructose-6-phosphate aminotransferase is a protein having glutamine-fructose-6-phosphate aminotransferase activity and that
   (i) comprises a polypeptide sequence according to any one of SEQ ID NOs: 39, 40, 41, 42, 43, 44, 45, 46, 47, 48, 49, 50, 51, 52 or 53 that differs from SEQ ID NO: 01 by a V12L, a Q96H, a Q157R and/or an E343V mutation, or
   (ii) is a functional homolog, variant or derivative of any one of SEQ ID NOs: 01, 02, 03, 04, 05, 06, 07, 08, 09, 10, 11, 12, 13, 14, 15, 16, 17, 18, 19, 20, 21, 22, 23, 24, 25, 26, 27, 28, 29, 30, 31, 32, 33, 34, 35, 36, 37 or 38 having at least 80% overall sequence identity to the full-length of any one of the polypeptides with SEQ ID NOs: 01, 02, 03, 04, 05, 06, 07, 08, 09, 10, 11, 12, 13, 14, 15, 16, 17, 18, 19, 20, 21, 22, 23, 24, 25, 26, 27, 28, 29, 30, 31, 32, 33, 34, 35, 36, 37 or 38, respectively, or
   (iii) is a functional homolog, variant or derivative of any one of SEQ ID NOs: 01, 02, 03, 04, 05, 06, 07, 08, 09, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52 or 53 having at least 80% overall sequence identity to the full-length of any one of the polypeptides with SEQ ID NOs: 01, 02, 03, 04, 05, 06, 07, 08, 09, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52 or 53, respectively, and differing from SEQ ID NO: 01 by a V12L, a Q96H, a Q157R and/or an E343V mutation.
4. Cell according to any one of previous embodiments, wherein at least one of the monosaccharide subunits is chosen from the list comprising N-acetylglucosamine, N-acetylmannosamine, N-acetylgalactosamine, 2-acetamido-2,6-dideoxy-L-arabino-4-hexulose, 2-acetamido-2,6-dideoxy-L-lyxo-4-hexulose, N-acetyl-L-rhamnosamine, N-acetyl-D-fucosamine, N-acetyl-L-pneumosamine, N-acetylmuramic acid, N-acetyl-L-quinovosamine, N-acetylneuraminic acid, and N-glycolylneuraminic acid.
5. Cell according to any one of previous embodiments, wherein the cell further synthesizes a nucleotide-sugar chosen from the list comprising UDP-N-acetylgalactosamine (UDP-GalNAc), UDP-N-acetylmannosamine (UDP-ManNAc), UDP-glucose (UDP-Glc), UDP-galactose (UDP-Gal), GDP-mannose (GDP-Man), UDP-glucuronate, UDP-galacturonate, UDP-2-acetamido-2,6-dideoxy-L-arabino-4-hexulose, UDP-2-acetamido-2,6-dideoxy-L-lyxo-4-hexulose, UDP-N-acetyl-L-rhamnosamine (UDP-L-RhaNAc or UDP-2-acetamido-2,6-dideoxy-L-mannose), dTDP-N-acetylfucosamine, UDP-N-acetylfucosamine (UDP-L-FucNAc or UDP-2-acetamido-2,6-dideoxy-L-galactose), UDP-N-acetyl-L-pneumosamine (UDP-L-PneNAC or UDP-2-acetamido-2,6-dideoxy-L-talose), UDP-N-acetylmuramic acid, UDP-N-acetyl-L-quinovosamine (UDP-L-QuiNAc or UDP-2-acetamido-2,6-dideoxy-L-glucose), CMP-sialic acid (CMP-Neu5Ac), CMP-N-glycolylneuraminic acid (CMP-Neu5Gc), GDP-fucose (GDP-Fuc), GDP-rhamnose and UDP-xylose.
6. Cell according to any one of previous embodiments, wherein the glycosyltransferase is chosen from the list comprising fucosyltransferases, sialyltransferases, galactosyltransferases, glucosyltransferases, mannosyltransferases, N-acetylglucosaminyltransferases, N-acetylgalactosaminyltransferases, N-acetylmannosaminyltransferases, xylosyltransferases, glucuronyltransferases, galacturonyltransferases, glucosaminyltransferases, N-glycolylneuraminyltransferases, rhamnosyltransferases, N-acetylrhamnosyltransferases, UDP-4-amino-4,6-dideoxy-N-acetyl-beta-L-altrosamine transaminases, UDP-N-acetylglucosamine enolpyruvyl transferases and fucosaminyltransferases,
preferably, wherein the cell is modified in the expression or activity of the glycosyltransferase.
7. Cell according to any one of previous embodiments, wherein the cell further expresses any one or more of the enzymes chosen from the list comprising glucosamine 6-phosphate N-acetyltransferase, phosphoacetylglucosamine mutase, UDP-N-acetylglucosamine pyrophosphorylase, galactoside beta-1,3-N-acetylglucosaminyltransferase, UTP-glucose-1-phosphate uridylyltransferase, UDP-glucose 4-epimerase, N-acetylglucosamine beta-1,3-galactosyltransferase, N-acetylglucosamine beta-1,4-galactosyltransferase, lactose permease, UDP-N-acetylglucosamine 2-epimerase, N-acetylneuraminate synthase, N-acylneuraminate cytidylyltransferase, glucose-6-phosphate isomerase or UDP-2-acetamido-2,6-dideoxy-L-talose 2-epimerase,
preferably, wherein the cell is modified in the expression or activity of at least one of the enzymes.
8. Cell according to any one of previous embodiments, wherein the glycosylated product is chosen from the list comprising a mammalian milk di- or oligosaccharide, 0-antigen, enterobacterial common antigen (ECA), capsular polysaccharides, peptidoglycan (PG) and antigens of the human ABO blood group system.
9. Cell according to any one of previous embodiments, wherein the cell uses at least one precursor for the synthesis of the glycosylated product, preferably the cell uses two or more precursors for the synthesis of the glycosylated product.
10. Cell according to any one of previous embodiments, wherein the cell is producing at least one precursor for the synthesis of the glycosylated product.
11. Cell according to any one of embodiment 9 or 10, wherein the precursor for the synthesis of the glycosylated product is completely converted into the glycosylated product.
12. Cell according to any one of previous embodiments, wherein the cell excretes at least one di- or oligosaccharide over the cytoplasm membrane.
13. Cell according to any one of previous embodiments, wherein the cell is selected from the group comprising microorganism, plant, or animal cells, preferably the microorganism is a bacterium, fungus or a yeast, preferably the plant is a rice, cotton, rapeseed, soy, maize or corn plant, preferably the animal is an insect, fish, bird or non-human mammal, preferably the animal cell is a mammalian cell line.
14. Cell according to embodiment 13, wherein the cell is a cell of a bacterium, preferably of an Escherichia coli strain, more preferably of an Escherichia coli strain that is a K-12 strain, even more preferably the Escherichia coli K-12 strain is E. coli MG1655.
15. Cell according to embodiment 13, wherein the cell is a yeast, preferably a yeast cell belonging to a genus chosen from the group comprising Saccharomyces, Zygosaccharomyces, Pichia, Komagataella, Hansenula, Kluyveromyces or Debaromyces.
16. Cell according to embodiment 13, wherein the cell is a fungus, preferably a fungus belonging to the genus Rhizopus, Dictyostelium, Penicillium, Mucor or Aspergillus.
17. A method to produce a glycosylated product comprising a di- or oligosaccharide that is composed of at least two different monosaccharide subunits by a cell, the method comprising the steps of:
   (a) providing a cell according to any one of embodiments 1 to 16, and
   (b) cultivating the cell under conditions permissive to produce the glycosylated product,
   (c) preferably, separating the glycosylated product from the cultivation.
18. Method according to embodiment 17, wherein during the cultivation the cell excretes the glycosylated product in the fermentation broth over the cytoplasm membrane.
19. Method according to any one of embodiments 17 or 18, wherein the separation comprises at least one of the following steps: clarification, ultrafiltration, nanofiltration, reverse osmosis, microfiltration, activated charcoal or carbon treatment, tangential flow high-performance filtration, tangential flow ultrafiltration, affinity chromatography, ion exchange chromatography, hydrophobic interaction chromatography and/or gel filtration, ligand exchange chromatography.
20. Method according to any one of embodiments 17 to 19, further comprising purification of the glycosylated product from the cell.
21. Method according to any one of embodiments 17 to 20, wherein the purification comprises at least one of the following steps: use of activated charcoal or carbon, use of charcoal, nanofiltration, ultrafiltration or ion exchange, use of alcohols, use of aqueous alcohol mixtures, crystallization, evaporation, precipitation, drying, spray drying or lyophilization.
22. A yeast or fungal cell that excretes a di- or oligosaccharide over the cytoplasm membrane.
23. Yeast or fungal cell according to embodiment 22 wherein the di- or oligosaccharide comprises at least one monosaccharide unit chosen from the list comprising N-acetylglucosamine, N-acetylmannosamine, N-acetylgalactosamine, 2-acetamido-2,6-dideoxy-L-arabino-4-hexulose, 2-acetamido-2,6-dideoxy-L-lyxo-4-hexulose, N-acetyl-L-rhamnosamine, N-acetyl-D-fucosamine, N-acetyl-L-pneumosamine, N-acetylmuramic acid, N-acetyl-L-quinovosamine, N-acetylneuraminic acid, or N-glycolylneuraminic acid.
24. Yeast or fungal cell according to any one of embodiment 22 or 23, wherein the cell is a metabolically engineered cell according to any one of embodiments 1 to 16.
25. Use of a cell according to any one of embodiments 1 to 16 and 22 to 24 for the production of a glycosylated product.
26. Use of a method according to any one of embodiments 17 to 21 for the production of a glycosylated product.

Moreover, this disclosure relates to following preferred specific embodiments:
1. A metabolically engineered cell for production of a glycosylated product comprising a di- or oligosaccharide that is composed of at least two different monosaccharide subunits, wherein the cell:
   (i) is capable to express, preferably expresses a variant yeast or fungal glutamine-fructose-6-phosphate aminotransferase, and
   (ii) is capable to synthesize, preferably synthesizes UDP-N-acetylglucosamine (UDP-GlcNAc), and
   (iii) is capable to express, preferably expresses a glycosyltransferase, and
   wherein the cell uses the UDP-GlcNAc to produce the glycosylated product.
2. Cell according to preferred embodiment 1, wherein the cell is a metabolically engineered cell modified with at least one gene expression module comprising the variant yeast or fungal glutamine-fructose-6-phosphate aminotransferase, preferably the expression from the expression module is constitutive or conditional upon non-chemical induction or repression.
3. Cell according to any one of previous preferred embodiments, wherein the variant yeast or fungal glutamine-fructose-6-phosphate aminotransferase is a protein having glutamine-fructose-6-phosphate aminotransferase activity and that:
   (i) comprises a polypeptide sequence according to any one of SEQ ID NOs: 39, 40, 41, 42, 43, 44, 45, 46, 47, 48, 49, 50, 51, 52 or 53 that differs from SEQ ID NO: 01 by a V12L, a Q96H, a Q157R and/or an E343V mutation, or
   (ii) is a polypeptide comprising or consisting of an amino acid sequence that is at least 80.0% sequence identical over a stretch of at least 200 amino acid residues to the amino acid sequence of any one of SEQ ID NOs: 01, 02, 03, 04, 05, 06, 07, 08, 09, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52 or 53, respectively, and that differs from SEQ ID NO: 01 by a V12L, a Q96H, a Q157R and/or an E343V mutation.
4. Cell according to any one of previous preferred embodiments, wherein at least one of the monosaccharide subunits is chosen from the list comprising N-acetylglucosamine, N-acetylmannosamine, N-acetylgalactosamine, 2-acetamido-2,6-dideoxy-L-arabino-4-hexulose, 2-acetamido-2,6-dideoxy-L-lyxo-4-hexulose, N-acetyl-L-rhamnosamine, N-acetyl-D-fucosamine, N-acetyl-L-pneumosamine, N-acetylmuramic acid, N-acetyl-L-quinovosamine, N-acetylneuraminic acid, and N-glycolylneuraminic acid.
5. Cell according to any one of previous preferred embodiments, wherein the cell further synthesizes a nucleotide-sugar chosen from the list comprising UDP-N-acetylgalactosamine (UDP-GalNAc), UDP-N-acetylmannosamine (UDP-ManNAc), UDP-glucose (UDP-Glc), UDP-galactose (UDP-Gal), GDP-mannose (GDP-Man), UDP-glucuronate, UDP-galacturonate, UDP-2-acetamido-2,6-dideoxy-L-arabino-4-hexulose, UDP-2-acetamido-2,6-dideoxy-L-lyxo-4-hexulose, UDP-N-acetyl-L-rhamnosamine (UDP-L-RhaNAc or UDP-2-acetamido-2,6-dideoxy-L-mannose), dTDP-N-acetylfucosamine, UDP-N-acetylfucosamine (UDP-L-FucNAc or UDP-2-acetamido-2,6-dideoxy-L-galactose), UDP-N-acetyl-L-pneumosamine (UDP-L-PneNAC or UDP-2-acetamido-2,6-dideoxy-L-talose), UDP-N-acetylmuramic acid, UDP-N-acetyl-L-quinovosamine (UDP-L-QuiNAc or UDP-2-acetamido-2,6-dideoxy-L-glucose), CMP-sialic acid (CMP-Neu5Ac), CMP-N-glycolylneuraminic acid (CMP-Neu5Gc), GDP-fucose (GDP-Fuc), GDP-rhamnose and UDP-xylose.
6. Cell according to any one of previous preferred embodiments, wherein the glycosyltransferase is chosen from the list comprising fucosyltransferases, sialyltransferases, galactosyltransferases, glucosyltransferases, mannosyltransferases, N-acetylglucosaminyltransferases, N-acetylgalactosaminyltransferases, N-acetylmannosaminyltransferases, xylosyltransferases, glucuronyltransferases, galacturonyltransferases, glucosaminyltransferases, N-glycolylneuraminyltransferases, rhamnosyltransferases, N-acetylrhamnosyltransferases, UDP-4-amino-4,6-dideoxy-N-acetyl-beta-L-altrosamine transaminases, UDP-N-acetylglucosamine enolpyruvyl transferases and fucosaminyltransferases,
   preferably, the fucosyltransferase is chosen from the list comprising alpha-1,2-fucosyltransferase, alpha-1,3-fucosyltransferase, alpha-1,3/4-fucosyltransferase, alpha-1,4-fucosyltransferase and alpha-1,6-fucosyltransferase,
   preferably, the sialyltransferase is chosen from the list comprising alpha-2,3-sialyltransferase, alpha-2,6-sialyltransferase and alpha-2,8-sialyltransferase,
   preferably, the galactosyltransferase is chosen from the list comprising beta-1,3-galactosyltransferase, N-acetylglucosamine beta-1,3-galactosyltransferase, beta-1,4-galactosyltransferase, N-acetylglucosamine beta-1,4-galactosyltransferase, alpha-1,3-galactosyltransferase and alpha-1,4-galactosyltransferase,
   preferably, the glucosyltransferase is chosen from the list comprising alpha-glucosyltransferase, beta-1,2-glucosyltransferase, beta-1,3-glucosyltransferase and beta-1,4-glucosyltransferase,
   preferably, the mannosyltransferase is chosen from the list comprising alpha-1,2-mannosyltransferase, alpha-1,3-mannosyltransferase and alpha-1,6-mannosyltransferase,
   preferably, the N-acetylglucosaminyltransferase is chosen from the list comprising galactoside beta-1,3-N-acetylglucosaminyltransferase and beta-1,6-N-acetylglucosaminyltransferase,
   preferably, the N-acetylgalactosaminyltransferase is an alpha-1,3-N-acetylgalactosaminyltransferase,
   preferably, wherein the cell is modified in the expression or activity of the glycosyltransferase.
7. Cell according to any one of previous preferred embodiments, wherein the cell further expresses any one or more of the enzymes chosen from the list comprising glucosamine 6-phosphate N-acetyltransferase, phosphoacetylglucosamine mutase, UDP-N-acetylglucosamine pyrophosphorylase, galactoside beta-1,3-N-acetylglucosaminyltransferase, UTP-glucose-1-phosphate uridylyltransferase, UDP-glucose 4-epimerase, N-acetylglucosamine beta-1,3-galactosyltransferase, N-acetylglucosamine beta-1,4-galactosyltransferase, lactose permease, UDP-N-acetylglucosamine 2-epimerase, N-acetylneuraminate synthase, N-acylneuraminate cytidylyltransferase, glucose-6-phosphate isomerase or UDP-2-acetamido-2,6-dideoxy-L-talose 2-epimerase,
preferably, wherein the cell is modified in the expression or activity of at least one of the enzymes.
8. Cell according to any one of previous preferred embodiments, wherein the glycosylated product is chosen from the list comprising a mammalian milk di- or oligosaccharide, preferably a human milk di- or oligosaccharide, 0-antigen, enterobacterial common antigen (ECA), the oligosaccharide repeats present in capsular polysaccharides, peptidoglycan (PG) and antigens of the human ABO blood group system.
9. Cell according to any one of previous preferred embodiments, wherein the cell uses at least one precursor for the synthesis of the glycosylated product, preferably the cell uses two or more precursors for the synthesis of the glycosylated product.
10. Cell according to any one of previous preferred embodiments, wherein the cell is producing at least one precursor for the synthesis of the glycosylated product.
11. Cell according to any one of preferred embodiment 9 or 10, wherein the precursor for the synthesis of the glycosylated product is completely converted into the glycosylated product.
12. Cell according to any one of previous preferred embodiments, wherein the cell excretes at least one di- or oligosaccharide out of the cell.
13. Cell according to any one of previous preferred embodiments, wherein the cell is a bacterium, fungus, yeast, a plant cell, an animal cell, or a protozoan cell,
   preferably the bacterium is an Escherichia coli strain, more preferably an Escherichia coli strain that is a K-12 strain, even more preferably the Escherichia coli K-12 strain is E. coli MG1655,
   preferably the fungus belongs to a genus chosen from the group comprising Rhizopus, Dictyostelium, Penicillium, Mucor or Aspergillus,
   preferably the yeast belongs to a genus chosen from the group comprising Saccharomyces, Zygosaccharomyces, Pichia, Komagataella, Hansenula, Kluyveromyces or Debaromyces,
   preferably the plant cell is an algal cell or is derived from tobacco, alfalfa, rice, tomato, cotton, rapeseed, soy, maize, or corn plant,
   preferably the animal cell is derived from non-human mammals, birds, fish, invertebrates, reptiles, amphibians or insects or is a genetically modified cell line derived from human cells excluding embryonic stem cells, more preferably the human and non-human mammalian cell is an epithelial cell, an embryonic kidney cell, a fibroblast cell, a COS cell, a Chinese hamster ovary (CHO) cell, a murine myeloma cell, an NIH-3T3 cell, a non-mammary adult stem cell or derivatives thereof, more preferably the insect cell is derived from Spodoptera frugiperda, Bombyx mori, Mamestra brassicae, Trichoplusia ni or Drosophila melanogaster,
   preferably the protozoan cell is a Leishmania tarentolae cell.
14. A method to produce a glycosylated product comprising a di- or oligosaccharide that is composed of at least two different monosaccharide subunits by a cell, the method comprising the steps of:
   (a) providing a cell according to any one of preferred embodiments 1 to 13, and
   (b) cultivating the cell under conditions permissive to produce the glycosylated product,
   (c) preferably, separating the glycosylated product from the cultivation.
15. Method according to preferred embodiment 14, wherein during the cultivation the cell excretes the glycosylated product out of the cell.
16. Method according to any one of preferred embodiments 14 or 15, wherein the separation comprises at least one of the following steps: clarification, ultrafiltration, nanofiltration, two-phase partitioning, reverse osmosis, microfiltration, activated charcoal or carbon treatment, treatment with non-ionic surfactants, enzymatic digestion, tangential flow high-performance filtration, tangential flow ultrafiltration, affinity chromatography, ion exchange chromatography, hydrophobic interaction chromatography and/or gel filtration, ligand exchange chromatography, electrodialysis.
17. Method according to any one of preferred embodiments 14 to 16, further comprising purification of the glycosylated product from the cell.
18. Method according to preferred embodiment 17, wherein the purification comprises at least one of the following steps: use of activated charcoal or carbon, use of charcoal, nanofiltration, ultrafiltration, electrophoresis, enzymatic treatment or ion exchange, temperature adjustment, pH adjustment, pH adjustment with an alkaline or acidic solution, use of alcohols, use of aqueous alcohol mixtures, crystallization, evaporation, precipitation, drying, spray drying, lyophilization, spray freeze drying, freeze spray drying, band drying, belt drying, vacuum band drying, vacuum belt drying, drum drying, roller drying, vacuum drum drying or vacuum roller drying.
19. A yeast or fungal cell that excretes a di- or oligosaccharide out of the cell.
20. Yeast or fungal cell according to preferred embodiment 19 wherein the di- or oligosaccharide comprises at least one monosaccharide unit chosen from the list comprising N-acetylglucosamine, N-acetylmannosamine, N-acetylgalactosamine, 2-acetamido-2,6-dideoxy-L-arabino-4-hexulose, 2-acetamido-2,6-dideoxy-L-lyxo-4-hexulose, N-acetyl-L-rhamnosamine, N-acetyl-D-fucosamine, N-acetyl-L-pneumosamine, N-acetylmuramic acid, N-acetyl-L-quinovosamine, N-acetylneuraminic acid, or N-glycolylneuraminic acid.
21. Yeast or fungal cell according to any one of preferred embodiment 19 or 20, wherein the cell is a metabolically engineered cell according to any one of preferred embodiments 1 to 13.
22. A vector comprising an isolated nucleic acid molecule encoding a variant yeast or fungal glutamine-fructose-6-phosphate aminotransferase of any one of preferred embodiments 1 and 3.
23. Use of a cell according to any one of preferred embodiments 1 to 13 and 19 to 21 for the production of a glycosylated product that is derived from UDP-GlcNAc and comprising a di- or oligosaccharide that is composed of at least two different monosaccharide subunits.
24. Use of a method according to any one of preferred embodiments 14 to 18 for the production of a glycosylated product that is derived from UDP-GlcNAc and comprising a di- or oligosaccharide that is composed of at least two different monosaccharide subunits.
25. Use of an isolated nucleic acid molecule encoding a variant yeast or fungal glutamine-fructose-6-phosphate aminotransferase of any one of preferred embodiments 1 and 3 for the production of a glycosylated product that is derived from UDP-GlcNAc and comprising a di- or oligosaccharide that is composed of at least two different monosaccharide subunits.
26. Use of a vector according to preferred embodiment 22 for the production of a glycosylated product that is derived from UDP-GlcNAc and comprising a di- or oligosaccharide that is composed of at least two different monosaccharide subunits.

The disclosure will be described in more detail in the examples.

The following examples will serve as further illustration and clarification of this disclosure and are not intended to be limiting.

### EXAMPLES

### Example 1. Materials and Methods Saccharomyces cerevisiae

Media

S. cerevisiae strains were cultured in Synthetic Defined yeast medium with Complete Supplement mixture (SD CSM) or CSM drop-out (e.g., CSM-HIS or CSM-LEU) containing 6.7 g/L Yeast Nitrogen Base without amino acids (YNB w/o AA, Difco), 22 g/L glucose monohydrate (Riedel-De Haen) and the appropriate selective amino acid mixture (e.g., 0.79 g/L CSM or 0.77 g/L CSM-HIS, MP Biomedicals). Production experiments for 6′-sialyllactose (6′SL), lacto-N-triose II (LNT II) and lacto-N-neotetraose (LNnT) also contained 5 g/L lactose. Solid medium was obtained by adding 20 g/L agar noble (Difco).

Fermentation runs were also performed in Synthetic Defined yeast medium with Complete Supplement mixture (SD CSM) or in CSM drop-out medium containing 2% glucose as carbon source.

One Shot TOP10 Chemically Competent™ Escherichia coli (C404003, ThermoFisher Scientific), used for cloning procedures and for maintaining plasmids, were cultured using Lysogeny Broth (LB) comprising 10 g/L tryptone (Difco), 5 g/L yeast extract (Difco) and 5 g/L sodium chloride (VWR) at 37° C. while shaking at 200 rpm. Twelve g/L agar (Biokar Diagnostics) was added if solid medium was required. If necessary, the required antibiotic (100 μg/mL ampicillin or 25 μg/mL chloramphenicol) was added after autoclaving. Solid LB without sodium chloride and supplemented with 50 g/L sucrose (filter sterilized using a 0.22 μm PTFE filter) was used when counterselection for SacBR was desired.

All components were autoclaved separately at 121° C. for 21 min.

Plasmids

Expression plasmids for the expression of the native GFA1 from S. cerevisiae BY4742 (SEQ ID NO: 01), its variants with the double mutations Q96H and Q157R (SEQ ID NO: 42), with the quadruple mutations V12L, Q96H, Q157R and E343V (SEQ ID NO: 39) or with other adaptations (SEQ ID NOs: 40, 41, 43, 44, 45, 46, 47, 48, 49, 50, 51, 52, 53) and/or GFA1 orthologs chosen from SEQ ID NOs: 02 to 38, the lactose permease (LAC12) from Kluyveromyces lactis NRRL Y-1140 (UniProt ID P07921), the N-acetylglucosamine-6-phosphate 2-epimerase (neuC) from Campylobacter jejuni (UniProt ID AAK91727.1), the N-acetylneuraminate synthase (neuB) from E. coli (UniProt ID Q46675), the N-acylneuraminate cytidylyltransferase (neuA) from Campylobacter jejuni (UniProt ID Q93MP7), a polypeptide having alpha-2,6-sialyltransferase activity and composed of amino acid residues 108 to 497 of the alpha-2,6-sialyltransferase (a26ST) from Photobacterium damselae (UniProt ID O66375), the beta-1,3-N-acetylglucosaminyltransferase (lgtA) from Neisseria meningitidis (SEQ ID NO: 54) and the N-acetylglucosamine beta-1,4-galactosyltransferase (lgtB) from N. meningitidis (UniProt ID Q51116) were cloned via Golden Gate cloning or via a modified version of the Versatile Genetic Assembly System (VEGAS) (Kuijpers et al., Microb. Cell Fact. 12, 47 (2013); Mitchell et al., Nucleic Acids Res. 43, 6620-6630 (2015)). Table 1 shows an overview of the proteins used. Yeast promoters (pCCW12, pFBA1, pPAB1, pPGK1, pTEF, pTDH3) and terminators (tADH1, tENO1, tGuo1, tSynth14, tSynth17, tSynth18) were selected based on existing literature (Curran et al., ACS Synth. Biol. 4, 824-832 (2015); Lee et al., ACS Synth. Biol. 4, 975-986 (2015)). Auxotrophic markers HISS and LEU2 were obtained from pUG27 (Euroscarf, P30115) and pUG73 (Euroscarf, P30118), respectively. CEN6/ARS4 (pSH47, Euroscarf, P30119) or 211. (pEX2, BCCM, p2890) was selected as origin of replication to maintain plasmids in yeast. All parts were stored on carrier vectors in One Shot TOP10 Chemically Competent™ E. coli. Different antibiotic resistance markers were used on the distinct carrier or expression plasmids. Oligonucleotides and gBlocks were obtained from Integrated DNA Technologies (IDT). The codon usage was adapted to the expression host S. cerevisiae using the tools of the supplier. Expression plasmids and linear DNA fragments for in vivo assembly were transformed into S. cerevisiae according to the method of Gietz and Woods (Gietz and Woods, Methods Enzymol. 350, 87-96 (2002)). An overview of the expression plasmids used in distinct examples is given in Table 2.

TABLE 1
Overview of proteins with corresponding SEQ ID NOs or UniProt IDs used in this disclosure

| | | | | Country of origin of |
|---|---|---|---|---|
| | | | | digital sequence |
| | Name | Organism | Origin | information |
| SEQ ID NO | | | | |
| 01 | GFA1 | S. cerevisiae S288c | Synthetic | Unknown |
| 02 | GFA1 ortholog | S. pastorianus strain CBS 1483 | Synthetic | The Netherlands |
| 03 | GFA1 ortholog | S. cerevisiae YJM693 | Synthetic | USA |
| 04 | GFA1 ortholog | S. cerevisiae YJM1307 | Synthetic | USA |
| 05 | GFA1 ortholog | S. cerevisiae Pf-1 | Synthetic | Japan |
| 06 | GFA1 ortholog | S. cerevisiae YJM456 | Synthetic | USA |
| 07 | GFA1 ortholog | S. cerevisiae NYR20 | Synthetic | Japan |
| 08 | GFA1 ortholog | S. paradoxus CBS432 | Synthetic | Unknown |
| 09 | GFA1 ortholog | S. cerevisiae YJM1311 | Synthetic | USA |
| 10 | GFA1 ortholog | S. cerevisiae P-684 | Synthetic | Japan |
| 11 | GFA1 ortholog | S. cerevisiae strain S288C | Synthetic | Unknown |
| 12 | GFA1 ortholog | S. kudriavzevii IFO 1802 | Synthetic | Unknown |
| 13 | GFA1 ortholog | S. cerevisiae × S. kudriavzevii VIN7 | Synthetic | Unknown |
| 14 | GFA1 ortholog | S. pastorianus strain CBS 1483 | Synthetic | The Netherlands |
| 15 | GFA1 ortholog | Naumovozyma castellii CBS 4309 | Synthetic | Finland |
| 16 | GFA1 ortholog | Candida glabrata strain DSY562 | Synthetic | Switzerland |
| 17 | GFA1 ortholog | Candida glabrata strain BG2 | Synthetic | USA |
| 18 | GFA1 ortholog | Naumovozyma dairenensis CBS 421 | Synthetic | Japan |
| 19 | GFA1 ortholog | Torulaspora delbrueckii CBS 1146 | Synthetic | Unknown |
| 20 | GFA1 ortholog | Torulaspora globosa strain CBS764 | Synthetic | USA |
| 21 | GFA1 ortholog | Zygosaccharomyces rouxii BRC 110957 | Synthetic | Japan |
| 22 | GFA1 ortholog | Ashbya gossypii FDAG1 | Synthetic | USA |
| 23 | GFA1 ortholog | Lachancea quebecensis LAQU0 | Synthetic | Canada |
| 24 | GFA1 ortholog | Kazachstania naganishii CBS 8797 | Synthetic | Japan |
| 25 | GFA1 ortholog | Saccharomycesceae sp. ‘Ashbya aceri’ | Synthetic | USA |
| 26 | GFA1 ortholog | Lachancea fermentati | Synthetic | Unknown |
| 27 | GFA1 ortholog | Tetrapisispora blattae CBS 6284 | Synthetic | Germany |
| 28 | GFA1 ortholog | Zygosaccharomyces parabailii strain ATCC 60483 | Synthetic | The Netherlands |
| 29 | GFA1 ortholog | Zygosaccharomyces bailii ISA1307 | Synthetic | Unknown |
| 30 | GFA1 ortholog | Zygosaccharomyces parabailii strain ATCC 60483 | Synthetic | The Netherlands |
| 31 | GFA1 ortholog | Zygosaccharomyces mellis Ca-7 | Synthetic | Japan |
| 32 | GFA1 ortholog | Eremothecium cymbalariae DBVPG#7215 | Synthetic | Unknown |
| 33 | GFA1 ortholog | Kluyveromyces dobzhanskii CBS 2104 | Synthetic | USA |
| 34 | GFA1 ortholog | Kluyveromyces lactis strain NRRL Y-1140 | Synthetic | USA |
| 35 | GFA1 ortholog | Eremothecium sinecaudum strain ATCC 58844 | Synthetic | Canada |
| 36 | GFA1 ortholog | Kluyveromyces lactis strain CBS 2105 | Synthetic | USA |
| 37 | GFA1 ortholog | Kluyveromyces marxianus DMKU3-1042 | Synthetic | Japan |
| 38 | GFA1 ortholog | Tetrapisispora blattae CBS 6284 | Synthetic | Germany |
| 39 | GFA1 variant | Artificial sequence | Synthetic | N.A. |
| 40 | GFA1 variant | Artificial sequence | Synthetic | N.A. |
| 41 | GFA1 variant | Artificial sequence | Synthetic | N.A. |
| 42 | GFA1 variant | Artificial sequence | Synthetic | N.A. |
| 43 | GFA1 variant | Artificial sequence | Synthetic | N.A. |
| 44 | GFA1 variant | Artificial sequence | Synthetic | N.A. |
| 45 | GFA1 variant | Artificial sequence | Synthetic | N.A. |
| 46 | GFA1 variant | Artificial sequence | Synthetic | N.A. |
| 47 | GFA1 variant | Artificial sequence | Synthetic | N.A. |
| 48 | GFA1 variant | Artificial sequence | Synthetic | N.A. |
| 49 | GFA1 variant | Artificial sequence | Synthetic | N.A. |
| 50 | GFA1 variant | Artificial sequence | Synthetic | N.A. |
| 51 | GFA1 variant | Artificial sequence | Synthetic | N.A. |
| 52 | GFA1 variant | Artificial sequence | Synthetic | N.A. |
| 53 | GFA1 variant | Artificial sequence | Synthetic | N.A. |
| 54 | lgtA | Neisseria meningitidis | Synthetic | United Kingdom |
| 55 | neuA | Pasteurella multocida | Synthetic | USA |
| 56 | NmeniST3 | Neisseria meningitidis | Synthetic | United Kingdom |
| 57 | PmultST2 | Pasteurella multocida subsp. Multocida str. Pm70 | Synthetic | Unknown |
| UniProt ID | | | | |
| AAK91727.1 | neuC | Campylobacter jejuni | Synthetic | Canada |
| Q46675 | neuB | E. coli K-12 MG1655 | Synthetic | USA |
| Q93MP7 | neuA | Campylobacter jejuni | Synthetic | Canada |
| O66375 | a26ST | Photobacterium damselae | Synthetic | Japan |
| P07921 | lac12 | Kluyveromyces lactis NRRL Y-1140 | Synthetic | USA |
| Q51116 | lgtB | Neisseria meningitidis | Synthetic | United Kingdom |
| P43577 | GNA1 | S. cerevisiae | Synthetic | USA |
| P38628 | PCM1 | S. cerevisiae | Synthetic | USA |
| P43123 | QRI1 | S. cerevisiae | Synthetic | USA |
| E0NCD4 | neuB | Neisseria meningitidis | Synthetic | United Kingdom |
| P31120 | glmM | E. coli K-12 MG1655 | Synthetic | USA |
| P0ACC7 | glmU | E. coli K-12 MG1655 | Synthetic | USA |
| Q9CLP3 | PmultST3 | Pasteurella multocida | Synthetic | USA |
| A8QYL1 | P-JT-ISH-224-ST6 | Photobacterium sp. JT-ISH-224 | Synthetic | Japan |
| P02920 | LacY | E. coli K-12 MG1655 | Synthetic | USA |
| E0IXR1 | cscB | E. coli W | Synthetic | USA |
| Q03417 | Frk | Zymomonas mobilis | Synthetic | United Kingdom |
| A0ZZH6 | BaSP | Bifidobacterium adolescentis | Synthetic | Germany |
| D3QY14 | wbgO | E. coli O55:H7 | Synthetic | Germany |
| P09147 | galE | E. coli K-12 MG1655 | Synthetic | USA |

TABLE 2
Overview of plasmids used in distinct examples of this disclosure

| Plasmid | Description |
|---|---|
| pMan01 | Centromeric plasmid, HIS5, pPGK1-neuC-tADH1 |
| pMan02 | Centromeric plasmid, HIS5, pPGK1-neuC-tADH1, pCCW12-GFA1-tSynth14 |
| pMan03 | Centromeric plasmid, HIS5, pPGK1-neuC-tADH1, pCCW12-GFA1^{Q96H Q157R}-tSynth14 |
| pMan04 | Centromeric plasmid, HIS5, pPGK1-neuC-tADH1, pCCW12-GFA1^{V12L Q96H Q157R 3343V}- |
| | tSynth14 |
| pMan05 | Centromeric plasmid, HIS5, pCCW12-GFA1-tSynth14 |
| pMan06 | Centromeric plasmid, HIS5, negative control plasmid |
| pNeu5Ac01 | Centromeric plasmid, HIS5, pPGK1-neuC-tADH1, pTDH3-neuB-tSynth18 |
| pNeu5Ac02 | Centromeric plasmid, HIS5, pPGK1-neuC-tADH1, pTDH3-neuB-tSynth18, pCCW12-GFA1- |
| | tSynth14 |
| pNeu5Ac03 | Centromeric plasmid, HIS5, pPGK1-neuC-tADH1, pTDH3-neuB-tSynth18, pCCW12- |
| | GFA1^{Q96H Q157R}-tSynth14 |
| pNeu5Ac04 | Centromeric plasmid, HIS5, pPGK1-neuC-tADH1, pTDH3-neuB-tSynth18, pCCW12- |
| | GFA1^{V12L Q96H Q157R 3343V}-tSynth14 |
| pSL6 | Centromeric plasmid, LEU2, pTEF-neuA-tGuo1, pFBA1-a26ST-tENO1, pPAB1-LAC12- |
| | tSynth14 |
| pTri01 | 2μ plasmid, HIS5, pTDH3-lgtA-tSynth18 |
| pTri02 | Centromeric plasmid, LEU2, pPAB1-LAC12-tSynth17 |
| pTri03 | Centromeric plasmid, LEU2, pCCW12-GFA1-tSynth14, pPAB1-LAC12-tSynth17 |
| pTri04 | Centromeric plasmid, LEU2, pCCW12-GFA1^{Q96H Q157R}-tSynth14, pPAB1-LAC12-tSynth17 |
| pTri05 | Centromeric plasmid, LEU2, pCCW12-GFA1^{V12L Q96H Q157R 3343V}-tSynth14, pPAB1-LAC12- |
| | tSynth17 |
| pTri06 | 2μ plasmid, HIS5, negative control plasmid |
| pTri07 | Centromeric plasmid, LEU2, negative control plasmid |
| pLNnT01 | 2μ plasmid, HIS5, pTDH3-lgtA-tSynth18, pTEF-lgtB-tADH1 |

Yeast Strains

Saccharomyces cerevisiae BY4742 (MATα, his3Δ1, leu2Δ0, lys2Δ0, ura3Δ0), derived from S. cerevisiae S288c, was obtained from the Euroscarf culture collection (Y10000, Euroscarf, University of Frankfurt, Germany) and was used as expression host. All S. cerevisiae strains were stored at −80° C. in cryovials with 30% sterile glycerol in a 1:1 ratio mixture.

TABLE 3
Overview of native and engineered S. cerevisiae strains used

| Strain | Genotype | Expression plasmid* | Expressed genes from plasmids |
|---|---|---|---|
| BY4742 | MATa, his3Δ1, | None | None |
| | leu2Δ0, lys2Δ0, | | |
| | ura3Δ0 | | |
| sMan01 | BY4742 | pMan01 | neuC |
| sMan02 | BY4742 | pMan02 | neuC + GFA1 |
| sMan03 | BY4742 | pMan03 | neuC + GFA1^{Q96H Q157R} |
| sMan04 | BY4742 | pMan04 | neuC + GFA1^{V12L Q96H Q157R E343V} |
| sMan05 | BY4742 | pMan05 | GFA1 |
| sMan06 | BY4742 | pMan06 | Empty plasmid |
| sNeu5Ac01 | BY4742 | pNeu5Ac01 | neuC + neuB |
| sNeu5Ac02 | BY4742 | pNeu5Ac02 | neuC + neuB + GFA1 |
| sNeu5Ac03 | BY4742 | pNeu5Ac03 | neuC + neuB + GFA1^{Q96H Q157R} |
| sNeu5Ac04 | BY4742 | pNeu5Ac04 | neuC + neuB + GFA1^{V12L Q96H Q157R E343V} |
| sSL06 | BY4742 | pNeu5Ac04 + pSL6 | neuC + neuB + GFA1^{V12L Q96H Q157R E343V} + |
| | | | neuA + a26ST + LAC12 |
| sLNTII_01 | BY4742 | pTri01 + pTri02 | lgtA + LAC12 |
| sLNTII_02 | BY4742 | pTri01 + pTri03 | lgtA + LAC12 + GFA1 |
| sLNTII_03 | BY4742 | pTri01 + pTri04 | lgtA + LAC12 + GFA1^{Q96H Q157R} |
| sLNTII_04 | BY4742 | pTri01 + pTri05 | lgtA + LAC12 + GFA1^{V12L Q96H Q157R E343V} |
| sLNTII_05 | BY4742 | pTri06 + pTri07 | Empty plasmids |
| sLNnT01 | BY4742 | pLNnT01 + pTri02 | lgtA + lgtB + LAC12 |
| sLNnT02 | BY4742 | pLNnT01 + pTri03 | lgtA + lgtB + LAC12 + GFA1 |
| sLNnT03 | BY4742 | pLNnT01 + pTri04 | lgtA + lgtB + LAC12 + GFA1^{Q96H Q157R} |
| sLNnT04 | BY4742 | pLNnT01 + pTri05 | lgtA + lgtB + LAC12 + GFA1^{V12L Q96H Q157R E343V} |

*See Table 2

Cultivation Conditions

Yeast cultures were inoculated from cryovial or plate in 5 mL of the appropriate medium using an inoculation needle and incubated overnight at 30° C. and 200 rpm. To obtain single colonies, required for growth and production experiments, strains were plated on (selective) SD CSM and incubated for 3 days at 30° C. Afterwards replicates were selected, cultured as pre-culture overnight in 5 mL and used to inoculate growth and production experiments at an optical density (OD) of 0.1. Growth and production experiments were performed in 250 mL shake flasks containing 50 mL (ManNAc, Neu5Ac and 6′ SL production) or 500 mL shake flasks containing 100 mL (LNT II and LNnT production) of fresh preheated (30° C.) appropriate medium and incubated at 30° C. and 200 rpm. Samples were collected at regular time points to evaluate growth and production.

A shake flask culture grown for 16 hours could also be used as inoculum for a bioreactor. Fermentations were carried out in a 5 L Biostat Dcu-B with a 4 L working volume, controlled by MFCS control software (Sartorius Stedim Biotech, Melsungen, Germany). Fermenters were inoculated with 4% inoculum. The temperature of the fermenters was maintained at 30° C. and pH was controlled between 5.5 and 6.5 with 20% ammonium hydroxide throughout the entire fermentations. During the initial hours of fermentation, aeration was controlled at 0.4 L/min, and dissolved oxygen controlled at 20% by agitation. During fed-batch the aeration was adjusted in a stepwise manner up to 1.5 L/min to maintain the dissolved oxygen levels. The exhaust gas was cooled. 10% solution of silicone antifoaming agent was added when foaming raised during the fermentation. The use of an inducer is not required since all genes are constitutively expressed. The fermentations were performed using a glucose feed; an additional lactose feed was used for production of LNT II, LNnT or 6′ SL. Regular samples were taken during fermentations.

To evaluate extracellular production, a 0.5 mL sample was centrifugated (11 000 rpm, 10 min) and supernatant was filtered through a PTFE filter (Novolab). To evaluate intracellular production, a 10 mL sample was collected and processed as described (Hollands et al., Metab. Eng. 52, 232-242 (2019). Herein, a 2 mL sample was centrifugated and the pellet was washed with dH₂O. The appropriate amount of Cellytic Y Cell Lysis Reagent (Sigma Aldrich) and acid-washed glass beads (425-600 μm; Sigma Aldrich) were added, whereupon the samples were vortexed in 10 cycles of 1 min vortexing at 4° C. and then put on ice for at least 30 sec. Last, the cells with beads were pelleted by centrifugating (10 000 rpm, 10 min) and supernatant was filtered through a PTFE filter.

Optical Density and pH

Cell density of the yeast cultures was monitored by measuring optical densities at 600 nm using a V-630 Bio Spectrophotometer (Jasco).

The pH of the filtered supernatant was measured to monitor potential changes during the growth and production experiment.

Analytical Analysis

Standards such as but not limited to sucrose, lactose, sialic acid, 6′-sialyllactose, LNT II, LNT and LNnT were purchased from Carbosynth (UK), Elicityl (France) and IsoSep (Sweden). Other compounds were analyzed with in-house made standards. N-acetylmannosamine (ManNAc), sialic acid (Neu5Ac) and 6′-sialyllactose (6′ SL) content in the filtered supernatant was quantified with a Waters Acquity UPLC H-class system connected to a UV-detector. A Rezex ROA-Organic Acid H+ column (100×4.6 mm ID, 8%) was used at 65° C. with 5 mM sulphuric acid as mobile phase. The flow rate was set to 0.1 mL/min and ManNAc, Neu5Ac and 6′ SL were measured at 200 nm using an ACQUITY TUV detector. Quantification was done based on quantified dilution ranges of standards. Lacto-N-triose II (LNT II) and lacto-N-neotetraose (LNnT) in the filtered supernatant were both derivatized prior to analysis. Derivatization was performed by adding anthranilamide (100 μL, 2.5 M), 2-picoline borane (100 μL, 0.6 M) and acetic acid (50 μL) to 250 μL of the sample, after which an incubation of 3 hours at 40° C. followed. Derivatized samples were quantified with a Waters Acquity UPLC H-class system connected to a UV-detector. An Acquity UPLC BEH Amide 1.7 μm column (21×100 mm) was used at 60° C. with a flow rate of 0.6 mL/min, following the gradient reported in Table 4. LNT II and LNnT were measured using an ACQUITY TUV detector using light of 254 nm. Quantifications were done based on quantified dilution ranges of standards.

TABLE 4
Gradient used for LNT II and LNnT separation. Eluent
A constitutes of 100 mM ammonium formate pH 4.4,
eluent B constitutes of 100% acetonitrile.

| Time (min) | Eluent A | Eluent B |
|---|---|---|
| 0 | 12 | 88 |
| 5 | 12 | 88 |
| 13 | 17 | 83 |
| 14 | 17 | 83 |
| 15.5 | 70 | 30 |
| 17.5 | 70 | 30 |
| 19 | 12 | 88 |

### Example 2. Evaluation of Engineered S. cerevisiae Strains for the Production of N-Acetylmannosamine (ManNAc)

The wild type S. cerevisiae BY4742 strain expressing the native genes GFA1 (SEQ ID NO: 01), GNA1 (UniProt ID P43577), PCM1 (UniProt ID P38628) and QRI1 (UniProt ID P43123) from its genome was transformed with expression plasmids comprising a constitutive transcriptional unit for neuC from C. jejuni (UniProt ID AAK91727.1) or an additional copy of the native GFA1 from S. cerevisiae BY4742 with SEQ ID NO: 01 or comprising constitutive transcriptional units for both neuC (UniProt ID AAK91727.1) and WT GFA1 with SEQ ID NO: 01, a variant GFA1 with SEQ ID NO: 42 (adapted by Q96H and Q157R compared to SEQ ID NO: 01) or a variant GFA1 with SEQ ID NO: 39 (adapted by V12L, Q96H, Q157R and E343V compared to SEQ ID NO: 01), resulting in strains sMan01 to sMan05 as enlisted in Table 3. The novel strains were evaluated and compared to a reference strain sMan06 (see Table 3) in a 7-days growth experiment according to the culture conditions provided in Example 1 using 250 mL shake flasks containing 50 mL of appropriate selective medium.

As shown in Table 5, ManNAc production could be confirmed in all engineered S. cerevisiae strains expressing neuC (sMan01, sMan02, sMan03 and sMan04). Strains sMan05 and sMan06 lacking neuC did not produce ManNAc (data not shown). In addition, Table 5 shows that an extra copy of WT GFA1 with SEQ ID NO: 01 presented on an expression plasmid additional to the native GFA1 present on the genome as is for sMan02 improved ManNAc production in the engineered strain and this production was about five times higher compared to a strain lacking the additional GFA1 as is for sMan01. Also, the expression of a variant GFA1 additional to the native GFA1 as is for sMan03 and sMan04 improved ManNAc production compared to the sMan01 strain lacking any GFA1 gene on plasmid. Herein, the GFA1 variant with the V12L, Q96H, Q157R and E343V mutations showed the highest and significant increase in production in this experiment at days 2, 3 and 4 compared to the GFA1 variant with the Q96H and Q157R mutations. The additional copy of WT GFA1 or a variant GFA1 (having the Q96H and Q157R mutations or having the V12L, Q96H, Q157R and E343V mutations) also had a significant positive effect on ManNAc productivity as shown in Table 6.

TABLE 5
Production of ManNAc (g/L) during a 7-days production experiment using
engineered S. cerevisiae strains sMan01 to sMan04 (see Table 3).

| Strain | Day 0 | Day 1 | Day 2 | Day 3 | Day 4 | Day 7 |
|---|---|---|---|---|---|---|
| sMan01 | 0.00 | 0.00 | 0.05 ± 0.01 | 0.08 ± 0.01 | 0.11 ± 0.01 | 0.24 ± 0.07 |
| sMan02 | 0.00 | 0.18 ± 0.01 | 0.44 ± 0.01 | 0.61 ± 0.02 | 0.79 ± 0.06 | 1.26 ± 0.10 |
| sMan03 | 0.00 | 0.18 ± 0.01 | 0.44 ± 0.01 | 0.58 ± 0.02 | 0.76 ± 0.04 | 1.13 ± 0.03 |
| sMan04 | 0.00 | 0.22 ± 0.01 | 0.61 ± 0.02 | 0.80 ± 0.06 | 0.93 ± 0.02 | 1.33 ± 0.06 |

TABLE 6
Productivity of ManNAc (g/L/h) during a 7-days
production experiment using engineered S. cerevisiae
strains sMan01 to sMan04 (see Table 3).

| Strain | Day 0-1 | Day 1-2 | Day 2-3 | Day 3-4 | Day 4-7 | Total |
|---|---|---|---|---|---|---|
| sMan01 | 0.000 | 0.002 | 0.001 | 0.001 | 0.002 | 0.001 |
| sMan02 | 0.007 | 0.011 | 0.007 | 0.007 | 0.006 | 0.008 |
| sMan03 | 0.007 | 0.011 | 0.006 | 0.007 | 0.005 | 0.007 |
| sMan04 | 0.009 | 0.016 | 0.008 | 0.005 | 0.006 | 0.008 |

### Example 3. Evaluation of Engineered S. cerevisiae Strains for the Production of Sialic Acid (Neu5Ac)

The wild type S. cerevisiae BY4742 strain expressing the native genes GFA1 (SEQ ID NO: 01), GNA1 (UniProt ID P43577), PCM1 (UniProt ID P38628) and QRI1 (UniProt ID P43123) from its genome was transformed with an expression plasmid comprising constitutive transcriptional units for neuC from C. jejuni (UniProt ID AAK91727.1) and neuB from E. co/i(UniProt ID Q46675) or comprising constitutive transcriptional units for neuC (UniProt ID AAK91727.1), neuB (UniProt ID Q46675) and either an additional copy of WT GFA1 from S. cerevisiae BY4742 with SEQ ID NO: 01 or a variant GFA1 with SEQ ID NO: 42 (adapted by Q96H and Q157R compared to SEQ ID NO: 01) or a variant GFA1 with SEQ ID NO: 39 (adapted by V12L, Q96H, Q157R and E343V compared to SEQ ID NO: 01), resulting in strains sNeu5Ac01 to sNeu5Ac04 (see Table 3). The novel strains were evaluated and compared to a reference strain sMan01 lacking neuB and the additional GFA1 copy in a 3-days growth experiment according to the culture conditions provided in Example 1 using 250 mL shake flasks containing 50 mL of appropriate selective medium.

As shown in Table 7, all newly created S. cerevisiae strains produced ManNAc with about 4.38 to 5.65 mg/L of ManNAc detected in the intracellular fractions, and between 114 and 240 mg/L ManNAc excreted from the producing cells to the cultivation. The presence of an additional copy of an GFA1 polypeptide (either native version or a variant containing two or four mutations) improved the extracellular production of ManNAc significantly, being more than two times higher compared to a strain lacking the additional GFA1 copy as is for sNeu5Ac01. Also, intracellular production of Neu5Ac ranging between 10.41 to 13.48 mg/L could be measured in the strains having an additional copy of native GFA1 or a variant GFA1. No Neu5Ac production could be detected in the sNeu5Ac01 strain only expressing one (native) GFA1 copy without an additional wild-type or variant GFA1.

TABLE 7
Production of ManNAc and Neu5Ac (mg/L) after 72 hours of
a production experiment using engineered S. cerevisiae strains
sNeu5Ac01 to sNeu5Ac04 (see Table 3). ManNAc was measured
both intracellularly (INTRA) as well as extracellularly
(EXTRA), Neu5Ac was only measured intracellularly.

| | ManNAc | ManNAc | Neu5Ac |
|---|---|---|---|
| Strain | (INTRA) | (EXTRA) | (INTRA) |
| sNeu5Ac01 | 4.38 ± 0.04 | 113.93 ± 0.67 | 0.00 |
| sNeu5Ac02 | 5.67 ± 0.06 | 239.52 ± 1.18 | 11.27 ± 1.16 |
| sNeu5Ac03 | 5.58 ± 0.07 | 228.23 ± 4.27 | 10.41 ± 0.25 |
| sNeu5Ac04 | 5.65 ± 0.09 | 238.99 ± 3.16 | 13.48 ± 0.78 |

### Example 4. Evaluation of Engineered S. cerevisiae Strains for the Production of 6′-Sialyllactose (6′ SL)

In a next step, the engineered S. cerevisiae strain sNeu5Ac04 expressing neuC (UniProt ID AAK91727.1), neuB (UniProt ID Q46675) and the GFA1 variant with SEQ ID NO: 39 (adapted by V12L, Q96H, Q157R and E343V compared to SEQ ID NO: 01) in addition to its native GFA1 with SEQ ID NO: 01 was transformed with an extra plasmid (pSL6) having constitutive transcriptional units for additional expression of neuA from C. jejuni (UniProt ID Q93MP7), LAC12 from K. lactis (UniProt ID P07921) and a polypeptide having alpha-2,6-sialyltransferase activity and composed of amino acid residues 108 to 497 of the alpha-2,6-sialyltransferase (a26ST) from Photobacterium damselae (UniProt ID O66375), resulting in strain sSL06 (see Table 3). When evaluated in a 3-days growth experiment according to the culture conditions provided in Example 1 using 250 mL shake flasks containing 50 mL of appropriate selective medium, the novel strain demonstrated to synthesize 13.71±0.26 mg/L 6′ SL extracellularly and 10.19±0.08 mg/L 6′ SL intracellularly after 72 hours of cultivation.

### Example 5. Evaluation of Engineered S. cerevisiae Strains for the Production of Lacto-N-Triose (LNT II) and/or Lacto-N-Neotetraose (LNnT)

The wild type S. cerevisiae BY4742 strain expressing the native genes GFA1 (SEQ ID NO: 01), GNA1 (UniProt ID P43577), PCM1 (UniProt ID P38628) and QRI1 (UniProt ID P43123) from its genome was transformed with expression plasmids comprising constitutive transcriptional units for lgtA from N. meningitidis with SEQ ID NO: 54 and LAC12 from K. lactis (UniProt ID P07921) or comprising constitutive transcriptional units for lgtA with SEQ ID NO: 54, LAC12 (UniProt ID P07921) and either an additional copy of the WT GFA1 from S. cerevisiae BY4742 with SEQ ID NO: 01 or a variant GFA1 with SEQ ID NO: 42 (adapted by Q96H and Q157R compared to SEQ ID NO: 01) or SEQ ID NO: 39 (adapted by V12L, Q96H, Q157R and E343V compared to SEQ ID NO: 01), resulting in strains sLNTII_01 to sLNTII_04 (see Table 3). In an alternative step, the wild type S. cerevisiae BY4742 strain was transformed with expression plasmids comprising constitutive transcriptional units for lgtA with SEQ ID NO: 54 and lgtB (UniProt ID Q51116), both originating from N. meningitidis, and LAC12 from K. lactis (UniProt ID P07921) or with expression plasmids comprising constitutive transcriptional units for lgtA with SEQ ID NO: 54, lgtB (UniProt ID Q51116), LAC12 (UniProt ID P07921) and either an additional copy of WT GFA1 from S. cerevisiae BY4742 with SEQ ID NO: 01 or a variant GFA1 with SEQ ID NO: 42 or SEQ ID NO: 39, resulting in strains sLNnT01 to sLNnT04 (see Table 3). As such, the sLNnT strains additionally expressed lgtB compared to the sLNTII strains.

All novel sLNTII_01 to sLNTII_04 and sLNnT01 to sLNnT04 strains were evaluated and compared to a reference strain sLNTII_05 (see Table 3) in a 3-days growth experiment according to the culture conditions provided in Example 1 using 500 mL shake flasks containing 100 mL of appropriate selective medium.

As shown in Table 8, all newly created S. cerevisiae strains produced and excreted LNT II or LNnT. Surprisingly, the presence of an additional copy of an GFA1 polypeptide (either native or a variant containing two or four mutations) improved the production of both LNT II and LNnT significantly. Herein, the sLNnT03 strain expressing the GFA1 variant with SEQ ID NO: 42 demonstrated to have the highest LNnT production.

TABLE 8
Extracellular production of LNT II or LNnT (mg/L)
after a 3-days production experiment using engineered S.
cerevisiae strains sLNTII_01 to sLNTII_04 and
sLNnT01 to sLNnT04, respectively (see Table 3).

| Strain | LNT II | Strain | LNnT |
|---|---|---|---|
| sLNTII_01 | 6.91 ± 16.60 | sLNnT01 | 0.85 ± 0.49 |
| sLNTII_02 | 140.82 ± 1.68 | sLNnT02 | 10.49 ± 0.09 |
| sLNTII_03 | 132.44 ± 4.69 | sLNnT03 | 22.94 ± 0.54 |
| sLNTII_04 | 132.27 ± 3.44 | sLNnT04 | 7.90 ± 0.08 |

### Example 6. Excretion of LNT II, LNnT and/or 6′ SL Using Engineered S. cerevisiae Strains

The wild type S. cerevisiae BY4742 strain expressing the native genes GFA1 (SEQ ID NO: 01), GNA1 (UniProt ID P43577), PCM1 (UniProt ID P38628) and QRI1 (UniProt ID P43123) from its genome was transformed with an expression plasmid comprising constitutive transcriptional units for neuC (UniProt ID AAK91727.1), neuB (UniProt ID Q46675) and a variant GFA1 with SEQ ID NO: 39 (adapted by V12L, Q96H, Q157R and E343V compared to SEQ ID NO: 01), resulting in strain sNeu5Ac04 (Table 3). In a next step, the engineered S. cerevisiae strain sNeu5Ac04 was transformed with an extra plasmid (pSL6) having constitutive transcriptional units for additional expression of neuA from C. jejuni (UniProt ID Q93MP7), LAC12 from K. lactis (UniProt ID P07921) and a polypeptide having alpha-2,6-sialyltransferase activity and composed of amino acid residues 108 to 497 of the alpha-2,6-sialyltransferase (a26ST) from Photobacterium damselae (UniProt ID O66375), resulting in strain sSL06 (see Table 3).

Furthermore, the wild type S. cerevisiae BY4742 was transformed with expression plasmids comprising constitutive transcriptional units for lgtA from N. meningitidis with SEQ ID NO: 54 and LAC12 from K. lactis (UniProt ID P07921) or comprising constitutive transcriptional units for lgtA with SEQ ID NO: 54, LAC12 (UniProt ID P07921) and either an additional copy of the WT GFA1 from S. cerevisiae BY4742 with SEQ ID NO: 01 or a variant GFA1 with SEQ ID NO: 42 (adapted by Q96H and Q157R compared to SEQ ID NO: 01) or a variant GFA1 with SEQ ID NO: 39 (adapted by V12L, Q96H, Q157R and E343V compared to SEQ ID NO: 01), resulting in strains sLNTII_01 to sLNTII_04 (see Table 3). In an alternative step, the wild type S. cerevisiae BY4742 strain was transformed with either expression plasmids comprising constitutive transcriptional units for lgtA with SEQ ID NO: 54 and lgtB (UniProt ID Q51116), both originating from N. meningitidis, and LAC12 from K. lactis (UniProt ID P07921) or with expression plasmids comprising constitutive transcriptional units for lgtA with SEQ ID NO: 54, lgtB (UniProt ID Q51116), LAC12 (UniProt ID P07921) and either an additional copy of WT GFA1 from S. cerevisiae BY4742 with SEQ ID NO: 01 or a variant GFA1 with SEQ ID NO: 42 or SEQ ID NO: 39, resulting in strains sLNnT01 to sLNnT04 (see Table 3).

All novel yeast strains were evaluated in a 3-days growth experiment according to the culture conditions provided in Example 1 using shake flasks containing appropriate selective medium.

As shown in Table 9, all newly created S. cerevisiae strains produced and excreted 6′ SL, LNT II or LNnT into the cultivation broth. Surprisingly, the presence of an additional copy of an GFA1 form improved the production of both LNT II and LNnT in the extracellular fraction significantly.

TABLE 9
Extracellular production of 6′SL, LNT II or LNnT
(mg/L) after a 3-days production experiment using engineered
S. cerevisiae strains (see Table 3).

| Strain | Product | Concentration (mg/L) |
|---|---|---|
| sSL06 | 6′SL | 13.71 ± 0.26 |
| sLNTII_01 | LNT II | 6.91 ± 16.60 |
| sLNTII_02 | LNT II | 140.82 ± 1.68 |
| sLNTII_03 | LNT II | 132.44 ± 4.69 |
| sLNTII_04 | LNT II | 132.27 ± 3.44 |
| sLNnT01 | LNnT | 0.85 ± 0.49 |
| sLNnT02 | LNnT | 10.49 ± 0.09 |
| sLNnT03 | LNnT | 22.94 ± 0.54 |
| sLNnT04 | LNnT | 7.90 ± 0.08 |

### Example 7. Evaluation of Engineered S. cerevisiae Strains for Production of ManNAc, Neu5Ac and 6′-Sialyllactose in a Batch Fermentation

Batch fermentations at bioreactor scale are performed to evaluate engineered S. cerevisiae strains expressing neuC from C. jejuni (UniProt ID AAK91727.1) and a variant GFA1, chosen from SEQ ID NO: 42 (adapted by Q96H and Q157R compared to SEQ ID NO: 01) or SEQ ID NO: 39 (adapted by V12L, Q96H, Q157R and E343V compared to SEQ ID NO: 01) and producing ManNAc like strains sManNAc03 and sManNAc04, or strains additionally expressing neuB from E. coli (UniProt ID Q46675) and producing ManNAc and Neu5Ac like strains sNeu5Ac03 and sNeu5Ac04, or strains additionally expressing neuB from E. coli (UniProt ID Q46675) and neuA from C. jejuni (UniProt ID Q93MP7), LAC12 from K. lactis (UniProt ID P07921) and a polypeptide having alpha-2,6-sialyltransferase activity and composed of amino acid residues 108 to 497 of the alpha-2,6-sialyltransferase (a26ST) from Photobacterium damselae (UniProt ID O66375) and producing ManNAc, Neu5Ac and 6′ SL like strain sSL06. Details of the engineered strains are summarized in Table 3. The bioreactor runs are performed as described in Example 1. In these examples, glucose is used as a carbon source. In the runs with the 6′ SL production strain lactose is added in the batch medium at a concentration ranging from 50 to 150 g/L as a precursor for 6′ SL formation. Regular samples are taken and the production of ManNAc, Neu5Ac and 6′ SL is measured as described in Example 1.

### Example 8. Evaluation of Engineered S. cerevisiae Strains for Production of LNT II and LNnT in a Batch Fermentation

In another example, batch fermentations at bioreactor scale are performed to evaluate engineered S. cerevisiae strains expressing lgtA with SEQ ID NO: 54 from N. meningitidis, LAC12 from K. lactis (UniProt ID P07921) and a variant GFA1, chosen from SEQ ID NO: 42 (adapted by Q96H and Q157R compared to SEQ ID NO: 01) or SEQ ID NO: 39 (adapted by V12L, Q96H, Q157R and E343V compared to SEQ ID NO: 01) and producing LNT II like strains sLNTII_03 and sLNTII_04, or strains additionally expressing lgtB from N. meningitidis (UniProt ID Q51116) and producing LNT II and LNnT like strains sLNnT03 and sLNnT04. Details of the engineered strains are summarized in Table 3. The bioreactor runs are performed as described in Example 1. In these examples, glucose is used as a carbon source. Lactose is added in the batch medium at a concentration ranging from 50 to 150 g/L as a precursor for LNT II and LNnT formation. Regular samples are taken and the production of LNT II and LNnT is measured as described in Example 1.

### Example 9. Evaluation of Engineered S. cerevisiae Strains for the Production of 3′-Sialyllactose (3′ SL)

Alternatively to Example 4, the engineered S. cerevisiae strain sNeu5Ac04 expressing neuC (UniProt ID AAK91727.1), neuB (UniProt ID Q46675) and the GFA1 variant with SEQ ID NO: 39 (adapted by V12L, Q96H, Q157R and E343V compared to SEQ ID NO: 01) in addition to its native GFA1 with SEQ ID NO: 01 is transformed with an extra plasmid having constitutive transcriptional units for additional expression of neuA from C. jejuni (UniProt ID Q93MP7), LAC12 from K. lactis (UniProt ID P07921) and a polypeptide having alpha-2,3-sialyltransferase activity and composed of amino acid residues 1 to 268 of the alpha-2,3-sialyltransferase (PmultST3) from Pasteurella multocida (UniProt ID Q9CLP3) or an alpha-2,3-sialyltransferase from N. meningitidis (NmeniST3) with SEQ ID NO: 56. The novel strain is evaluated for production of 3′SL when evaluated in a 3-days growth experiment according to the culture conditions provided in Example 1 using appropriate selective medium comprising lactose.

### Example 10. Evaluation of Engineered S. cerevisiae Strains for the Production of LNT II and Lacto-N-Tetraose (LNT)

Alternatively to Example 5, the wild type S. cerevisiae BY4742 strain expressing the native genes GFA1 (SEQ ID NO: 01), GNA1 (UniProt ID P43577), PCM1 (UniProt ID P38628) and QRI1 (UniProt ID P43123) from its genome is transformed with expression plasmids comprising constitutive transcriptional units for the beta-1,3-N-acetylglucosaminyltransferase lgtA from N. meningitidis with SEQ ID NO: 54, the N-acetylglucosamine beta-1,3-galactosyltransferase wbgO from E. coli O55:H7 (UniProt ID D3QY14), the lactose permease LAC12 from K. lactis NRRL Y-1140 (UniProt ID P07921) and the variant GFA1 with SEQ ID NO: 39 (adapted by V12L, Q96H, Q157R and E343V compared to SEQ ID NO: 01). The novel strain is evaluated for production of LNT II and lacto-N-tetraose (LNT) when evaluated in a 3-days growth experiment according to the culture conditions provided in Example 1 using appropriate selective medium comprising lactose.

### Example 11. Evaluation of Engineered S. cerevisiae Strains for the Production of an Oligosaccharide Mixture Comprising LNT II, Sialylated LNT II, LNT, 3′ SL and LSTa

A wild-type S. cerevisiae BY4742 strain expressing the native genes GFA1 (SEQ ID NO: 01), GNA1 (UniProt ID P43577), PCM1 (UniProt ID P38628) and QRI1 (UniProt ID P43123) from its genome is modified with genomic knock-ins of constitutive transcriptional units for the beta-1,3-N-acetylglucosaminyltransferase lgtA from N. meningitidis with SEQ ID NO: 54, the N-acetylglucosamine beta-1,3-galactosyltransferase wbgO from E. coli O55:H7 (UniProt ID D3QY14), N-acetylglucosamine-6-phosphate 2-epimerase (neuC) from C. jejuni (UniProt ID AAK91727.1), the N-acetylneuraminate synthase (neuB) from E. coli (UniProt ID Q46675)) and an GFA1 ortholog, chosen from SEQ ID NOs: 02 to 38 and/or a variant GFA1, chosen from SEQ ID NOs: 39 to 53. In a next step, the engineered strains are transformed with an expression plasmid having constitutive transcriptional units for additional expression of N-acylneuraminate cytidylyltransferase (neuA) from C. jejuni (UniProt ID Q93MP7), lactose permease LAC12 from K. lactis (UniProt ID P07921) and a polypeptide having alpha-2,3-sialyltransferase activity and composed of amino acid residues 1 to 268 of the alpha-2,3-sialyltransferase (PmultST3) from Pasteurella multocida (UniProt ID Q9CLP3) or an alpha-2,3-sialyltransferase from N. meningitidis (NmeniST3) with SEQ ID NO: 56. The novel strains are evaluated for production of an oligosaccharide mixture comprising LNT II, 3′-sialylated LNT II (Neu5Ac-a2,3-GlcNAc-b1,3-Gal-b1,4-Glc), LNT, 3′SL and LSTa (Neu5Ac-a2,3-Gal-b1,3-GlcNAc-b1,3-Gal-b1,4-Glc) when evaluated in a 3-days growth experiment according to the culture conditions provided in Example 1 using appropriate selective medium comprising lactose.

### Example 12. Evaluation of Engineered S. cerevisiae Strains for the Production of an Oligosaccharide Mixture Comprising LNT II, Sialylated LNT II, LNnT, 6′ SL and LSTc

A wild-type S. cerevisiae BY4742 strain expressing the native genes GFA1 (SEQ ID NO: 01), GNA1 (UniProt ID P43577), PCM1 (UniProt ID P38628) and QRI1 (UniProt ID P43123) from its genome is modified with genomic knock-ins of constitutive transcriptional units for the beta-1,3-N-acetylglucosaminyltransferase lgtA from N. meningitidis with SEQ ID NO: 54, the N-acetylglucosamine beta-1,4-galactosyltransferase (lgtB) from N. meningitidis (UniProt ID Q51116), N-acetylglucosamine-6-phosphate 2-epimerase (neuC) from C. jejuni (UniProt ID AAK91727.1), the N-acetylneuraminate synthase (neuB) from E. coli (UniProt ID Q46675) and an GFA1 ortholog, chosen from SEQ ID NOs: 02 to 38 and/or a variant GFA1, chosen from SEQ ID NOs: 39 to 53. In a next step, the engineered strains are transformed with an expression plasmid having constitutive transcriptional units for additional expression of N-acylneuraminate cytidylyltransferase (neuA) from C. jejuni (UniProt ID Q93MP7), lactose permease LAC12 from K. lactis (UniProt ID P07921) and a polypeptide having alpha-2,6-sialyltransferase activity and composed of amino acid residues 108 to 497 of the alpha-2,6-sialyltransferase (a26ST) from Photobacterium damselae (UniProt ID O66375) or a polypeptide having alpha-2,6-sialyltransferase activity and composed of amino acid residues 18 to 514 of the alpha-2,6-sialyltransferase from Photobacterium sp. JT-ISH-224 (UniProt ID A8QYL1). The novel strains are evaluated for production of an oligosaccharide mixture comprising LNT II, 6′-sialylated LNT II (Neu5Ac-a2,6-[GlcNAc-b1,3]-Gal-b1,4-Glc), LNnT, 6′SL and LSTc (Neu5Ac-a2,6-Gal-b1,4-GlcNAc-b1,3-Gal-b1,4-Glc) when evaluated in a 3-days growth experiment according to the culture conditions provided in Example 1 using appropriate selective medium comprising lactose.

### Example 13. Evaluation of Engineered S. cerevisiae Strains for the Production of an Oligosaccharide Mixture Comprising LNT II, Sialylated LNT II, LNnT, 3′ SL and LSTd

A wild-type S. cerevisiae BY4742 strain expressing the native genes GFA1 (SEQ ID NO: 01), GNA1 (UniProt ID P43577), PCM1 (UniProt ID P38628) and QRI1 (UniProt ID P43123) from its genome is modified with genomic knock-ins of constitutive transcriptional units for the beta-1,3-N-acetylglucosaminyltransferase lgtA from N. meningitidis with SEQ ID NO: 54, the N-acetylglucosamine beta-1,4-galactosyltransferase (lgtB) from N. meningitidis (UniProt ID Q51116), N-acetylglucosamine-6-phosphate 2-epimerase (neuC) from C. jejuni (UniProt ID AAK91727.1), the N-acetylneuraminate synthase (neuB) from E. coli (UniProt ID Q46675) and an GFA1 ortholog, chosen from SEQ ID NOs: 02 to 38 and/or a variant GFA1, chosen from SEQ ID NOs: 39 to 53. In a next step, the engineered strains are transformed with an expression plasmid having constitutive transcriptional units for additional expression of N-acylneuraminate cytidylyltransferase (neuA) from C. jejuni (UniProt ID Q93MP7), lactose permease LAC12 from K. lactis (UniProt ID P07921) and a polypeptide having alpha-2,3-sialyltransferase activity and composed of amino acid residues 1 to 268 of the alpha-2,3-sialyltransferase (PmultST3) from Pasteurella multocida (UniProt ID Q9CLP3) or an alpha-2,3-sialyltransferase from N. meningitidis (NmeniST3) with SEQ ID NO: 56. The novel strains are evaluated for production of an oligosaccharide mixture comprising LNT II, 3′-sialylated LNT II (Neu5Ac-a2,3-GlcNAc-b1,3-Gal-b1,4-Glc), LNnT, 3′ SL and LSTd (Neu5Ac-a2,3-Gal-b1,4-GlcNAc-b1,3-Gal-b1,4-Glc) when evaluated in a 3-days growth experiment according to the culture conditions provided in Example 1 using appropriate selective medium comprising lactose.

### Example 14. Materials and Methods Escherichia coli

Media

Two media were used to cultivate E. coli: i.e., Luria Broth (LB) and minimal medium. The LB medium comprised 1% tryptone peptone (Difco), 0.5% yeast extract (Difco) and 0.5% sodium chloride (VWR). The minimal medium used in the cultivation experiments in 96-well plates or in shake flasks contained 2.00 g/L NH₄Cl, 5.00 g/L (NH₄)₂SO₄, 2.993 g/L KH₂PO₄, 7.315 g/L K₂HPO₄, 8.372 g/L MOPS, 0.5 g/L NaCl, 0.5 g/L MgSO₄·7H₂O, 30 g/L sucrose or 30 g/L glycerol, 1 mL/L vitamin solution, 100 μl/L molybdate solution, and 1 mL/L selenium solution. As specified in the respective examples for production of 3′ SL, 6′ SL, LNT II and/or LNnT, 20 g/L lactose was additionally added to the medium as precursor. The minimal medium was set to a pH of 7.0 with 1M KOH. Vitamin solution comprised 3.6 g/L FeCl₂·4H₂O, 5 g/L CaCl₂·2H₂O, 1.3 g/L MnCl₂·2H₂O, 0.38 g/L CuCl₂·2H₂O, 0.5 g/L CoCl₂·6H₂O, 0.94 g/L ZnCl₂, 0.0311 g/L H₃BO₄, 0.4 g/L Na₂EDTA·2H₂O and 1.01 g/L thiamine·HCl. The molybdate solution contained 0.967 g/L NaMoO₄·2H₂O. The selenium solution contained 42 g/L Seo₂.

The minimal medium for fermentations contained 6.75 g/L NH₄Cl, 1.25 g/L (NH₄)₂SO₄, 2.93 g/L KH₂PO₄ and 7.31 g/L KH₂PO₄, 0.5 g/L NaCl, 0.5 g/L MgSO₄·7H₂O, 30 g/L sucrose or 30 g/L glycerol, 1 mL/L vitamin solution, 100 μL/L molybdate solution, and 1 mL/L selenium solution with the same composition as described above. As specified in the respective examples, 0.30 g/L sialic acid, 20 g/L lactose, 20 g/L LacNAc and/or 20 g/L LNB were additionally added to the medium as precursor(s).

Complex medium was sterilized by autoclaving (121° C., 21 min) and minimal medium by filtration (0.22 μm Sartorius). When necessary, the medium was made selective by adding an antibiotic: e.g., chloramphenicol (20 mg/L), carbenicillin (100 mg/L), spectinomycin (40 mg/L) and/or kanamycin (50 mg/L).

Plasmids

pKD46 (Red helper plasmid, Ampicillin resistance), pKD3 (contains an FRT-flanked chloramphenicol resistance (cat) gene), pKD4 (contains an FRT-flanked kanamycin resistance (kan) gene), and pCP20 (expresses FLP recombinase activity) plasmids were obtained from Prof. R. Cunin (Vrije Universiteit Brussel, Belgium in 2007). Plasmids were maintained in the host E. coli DH5alpha (F⁻, phi80dlacZΔM15, Δ(lacZYA-argF) U169, deoR, recA1, endA1, hsdR17(rk⁻, mk⁺), phoA, supE44, lambda⁻, thi-1, gyrA96, relA1) bought from Invitrogen.

Strains and Mutations

Escherichia coli K12 MG1655 [λ⁻, F⁻, rph-1] was obtained from the Coli Genetic Stock Center (US), CGSC Strain #: 7740, in March 2007. Gene disruptions, gene introductions and gene replacements were performed using the technique published by Datsenko and Wanner (PNAS 97 (2000), 6640-6645). This technique is based on antibiotic selection after homologous recombination performed by lambda Red recombinase. Subsequent catalysis of a flippase recombinase ensures removal of the antibiotic selection cassette in the final production strain. Transformants carrying a Red helper plasmid pKD46 were grown in 10 mL LB media with ampicillin, (100 mg/L) and L-arabinose (10 mM) at 30° C. to an OD₆₀₀ₙₘ of 0.6. The cells were made electrocompetent by washing them with 50 mL of ice-cold water, a first time, and with 1 mL ice cold water, a second time. Then, the cells were resuspended in 50 μL of ice-cold water. Electroporation was done with 50 μL of cells and 10-100 ng of linear double-stranded-DNA product by using a Gene Pulser™ (BioRad) (600 Ω, 25 μFD, and 250 volts). After electroporation, cells were added to 1 mL LB media incubated 1 h at 37° C., and finally spread onto LB-agar containing 25 mg/L of chloramphenicol or 50 mg/L of kanamycin to select antibiotic resistant transformants. The selected engineered strains were verified by PCR with primers upstream and downstream of the modified region and were grown in LB-agar at 42° C. for the loss of the helper plasmid. The engineered strains were tested for ampicillin sensitivity. The linear ds-DNA amplicons were obtained by PCR using pKD3, pKD4 and their derivates as template. The primers used had a part of the sequence complementary to the template and another part complementary to the side on the chromosomal DNA where the recombination must take place. For the genomic knockout, the region of homology was designed 50-nt upstream and 50-nt downstream of the start and stop codon of the gene of interest. For the genomic knock-in, the transcriptional starting point (+1) had to be respected. PCR products were PCR-purified, digested with Dpnl, re-purified from an agarose gel, and suspended in elution buffer (5 mM Tris, pH 8.0). Selected engineered strains were transformed with pCP20 plasmid, which is an ampicillin and chloramphenicol resistant plasmid that shows temperature-sensitive replication and thermal induction of FLP synthesis. The ampicillin-resistant transformants were selected at 30° C., after which a few were colony purified in LB at 42° C. and then tested for loss of all antibiotic resistance and of the FLP helper plasmid. The gene knock outs and knock ins are checked with control primers.

In an example for sialic acid (Neu5Ac) production, the engineered strain was derived from E. coli K12 MG1655 comprising knockouts of the E. coli nagA and nagB genes and genomic knock-ins of constitutive transcriptional units containing a phosphoglucosamine mutase like e.g., glmM from E. coli (UniProt ID P31120), a N-acetylglucosamine-1-phosphate uridyltransferase/glucosamine-1-phosphate acetyltransferase like e.g., glmU from E. coli (UniProt ID P0ACC7), an UDP-N-acetylglucosamine 2-epimerase like e.g., neuC from C. jejuni (UniProt ID AAK91727.1) and an N-acetylneuraminate synthase like e.g., neuB from N. meningitidis (UniProt ID E0NCD4). Sialic acid production can further be optimized in the engineered E. coli strain with genomic knockouts of the E. coli genes comprising nagC, nagD, nagE, nanA, nanE, nanK, manX, manY and manZ as described in WO18122225 and with genomic knock-ins of constitutive transcriptional units comprising an GFA1 ortholog, chosen from SEQ ID NOs: 02 to 38 and/or a variant GFA1, chosen from SEQ ID NOs: 39 to 53.

For sialylated oligosaccharide production, the sialic acid production strains further need to express an N-acylneuraminate cytidylyltransferase like e.g., neuA from Pasteurella multocida with SEQ ID NO: 55, and (i) a beta-galactoside alpha-2,3-sialyltransferase like e.g., PmultST3 from P. multocida (UniProt ID Q9CLP3), a PmultST3-like polypeptide comprising amino acid residues 1 to 268 of UniProt ID Q9CLP3 having beta-galactoside alpha-2,3-sialyltransferase activity, NmeniST3 from N. meningitidis (SEQ ID NO: 56) or PmultST2 from P. multocida subsp. Multocida str. Pm70 (SEQ ID NO: 57), (ii) a beta-galactoside alpha-2,6-sialyltransferase like e.g., PdST6 from Photobacterium damselae (UniProt ID O66375), a PdST6-like polypeptide comprising amino acid residues 108 to 497 of UniProt ID O66375 having beta-galactoside alpha-2,6-sialyltransferase activity, P-JT-ISH-224-ST6 from Photobacterium sp. JT-ISH-224 (UniProt ID A8QYL1) or a P-JT-ISH-224-ST6-like polypeptide comprising amino acid residues 18 to 514 of UniProt ID A8QYL1 having beta-galactoside alpha-2,6-sialyltransferase activity, and/or (iii) an alpha-2,8-sialyltransferase like e.g., from M. musculus (UniProt ID Q64689). Constitutive transcriptional units of the N-acylneuraminate cytidylyltransferase and the sialyltransferases can be delivered to the engineered strain either via genomic knock-in or via expression plasmids. If the engineered strains producing sialic acid and CMP-sialic acid were intended to make sialylated lactose structures, the strains were additionally modified with genomic knockouts of the E. coli LacZ, LacY and LacA genes and with a genomic knock-in of a constitutive transcriptional unit for a lactose permease like e.g., LacY from E. coli (UniProt ID P02920). All engineered strains producing sialic acid, CMP-sialic acid and/or sialylated oligosaccharides could optionally be adapted for growth on sucrose via genomic knock-ins of constitutive transcriptional units containing a sucrose transporter like e.g., CscB from E. coli W (UniProt ID E0IXR1), a fructose kinase like e.g., Frk originating from Zymomonas mobilis (UniProt ID Q03417) and a sucrose phosphorylase like e.g., originating from Bifidobacterium adolescentis (UniProt ID A0ZZH6).

In an example to produce LNT II and oligosaccharides originating thereof comprising lacto-N-tetraose (LNT) and lacto-N-neotetraose (LNnT), the engineered strain was derived from E. coli K12 MG1655 and modified with a knockout of the E. coli LacZ and nagB genes and with a genomic knock-in of a constitutive transcriptional unit for a galactoside beta-1,3-N-acetylglucosaminyltransferase like e.g., lgtA from N. meningitidis with SEQ ID NO: 54. For LNT or LNnT production, the engineered strain is further modified with constitutive transcriptional units for an N-acetylglucosamine beta-1,3-galactosyltransferase like e.g., wbgO from E. coli O55:H7 (UniProt ID D3QY14) or an N-acetylglucosamine beta-1,4-galactosyltransferase like e.g., lgtB from N. meningitidis (UniProt ID Q51116), respectively, that can be delivered to the strain either via genomic knock-in or from an expression plasmid. Optionally, multiple copies of the galactoside beta-1,3-N-acetylglucosaminyltransferase, N-acetylglucosamine beta-1,3-galactosyltransferase and/or N-acetylglucosamine beta-1,4-galactosyltransferase genes could be added to the engineered E. coli strains. Also, LNT and/or LNnT production can be enhanced by improved UDP-GlcNAc production by modification of the strains with one or more genomic knock-ins of a constitutive transcriptional unit for an GFA1 ortholog, chosen from SEQ ID NOs: 02 to 38 and/or a variant GFA1, chosen from SEQ ID NOs: 39 to 53. In addition, the strains can optionally be modified for enhanced UDP-galactose production with genomic knockouts of the E. coli ushA and galT genes. The engineered E. coli strains can also optionally be adapted with a genomic knock-in of a constitutive transcriptional unit for an UDP-glucose-4-epimerase like e.g., galE from E. coli (UniProt ID P09147), a phosphoglucosamine mutase like e.g., glmM from E. coli (UniProt ID P31120) and an N-acetylglucosamine-1-phosphate uridyltransferase/glucosamine-1-phosphate acetyltransferase like e.g., glmU from E. coli (UniProt ID P0ACC7). The engineered strains could also optionally be adapted for growth on sucrose via genomic knock-ins of constitutive transcriptional units containing a sucrose transporter like e.g., CscB from E. coli W (UniProt ID E0IXR1), a fructose kinase like e.g., Frk originating from Z. mobilis (UniProt ID Q03417) and a sucrose phosphorylase like e.g., originating from B. adolescentis (UniProt ID A0ZZH6).

Preferably but not necessarily, any one or more of the glycosyltransferases and the proteins involved in nucleotide-activated sugar synthesis are N- and/or C-terminally fused to a solubility enhancer tag like e.g., a SUMO-tag, an MBP-tag, His, FLAG, Strep-II, Halo-tag, NusA, thioredoxin, GST and/or the Fh8-tag to enhance their solubility (Costa et al., Front. Microbiol. 2014, doi.org/10.3389/fmicb.2014.00063; Fox et al., Protein Sci. 2001, 10(3), 622-630; Jia and Jeaon, Open Biol. 2016, 6: 160196).

Optionally, the engineered E. coli strains were modified with a genomic knock-ins of a constitutive transcriptional unit encoding a chaperone protein like e.g., DnaK, DnaJ, GrpE or the GroEL/ES chaperonin system (Baneyx F., Palumbo J. L. (2003) Improving Heterologous Protein Folding via Molecular Chaperone and Foldase Co-Expression. In: Vaillancourt P. E. (eds) E. coli Gene Expression Protocols. Methods in Molecular Biology™, vol 205. Humana Press).

All constitutive promoters, UTRs and terminator sequences originated from the libraries described by Cambray et al. (Nucleic Acids Res. 2013, 41(9), 5139-5148), Dunn et al. (Nucleic Acids Res. 1980, 8, 2119-2132), Edens et al. (Nucleic Acids Res. 1975, 2, 1811-1820), Kim and Lee (FEBS Letters 1997, 407, 353-356) and Mutalik et al. (Nat. Methods 2013, No. 10, 354-360). All genes were ordered synthetically at Twist Bioscience (twistbioscience.com) or IDT (eu.idtdna.com) and the codon usage was adapted using the tools of the supplier.

All strains were stored in cryovials at −80° C. (overnight LB culture mixed in a 1:1 ratio with 70% glycerol).

Cultivation Conditions

A preculture of 96-well microtiter plate experiments was started from a cryovial, in 150 μL LB and was incubated overnight at 37° C. on an orbital shaker at 800 rpm. This culture was used as inoculum for a 96 well square microtiter plate, with 400 μL minimal medium by diluting 400×. These final 96-well culture plates were then incubated at 37° C. on an orbital shaker at 800 rpm for 72h, or shorter, or longer. To measure sugar concentrations at the end of the cultivation experiment whole broth samples were taken from each well by boiling the culture broth for 15 min at 60° C. before spinning down the cells (=average of intra- and extracellular sugar concentrations).

A preculture for the bioreactor was started from an entire 1 mL cryovial of a certain strain, inoculated in 250 mL or 500 mL minimal medium in a 1 L or 2.5 L shake flask and incubated for 24 h at 37° C. on an orbital shaker at 200 rpm. A 5 L bioreactor was then inoculated (250 mL inoculum in 2 L batch medium); the process was controlled by MFCS control software (Sartorius Stedim Biotech, Melsungen, Germany). Culturing condition were set to 37° C., and maximal stirring; pressure gas flow rates were dependent on the strain and bioreactor. The pH was controlled at 6.8 using 0.5 M H₂SO₄ and 20% NH₄OH. The exhaust gas was cooled. 10% solution of silicone antifoaming agent was added when foaming raised during the fermentation.

Optical Density, pH and Analytical Analysis

The determination of the optical density and the pH of the bacterial cultures as well as the analytical analysis were performed as described in Example 1.

### Example 15. Evaluation of Engineered E. coli Strains for the Production of ManNAc and Neu5Ac and Either 3′-Sialyllactose (3′SL) or 6′-Sialyllactose (6′SL)

A wild-type E. coli K-12 MG1655 strain is modified with genomic knockouts of the E. coli genes nagA, nagB, lacY, lacZ, nanA, nanE and nanK together with genomic knock-ins of constitutive transcriptional units for an GFA1 ortholog, chosen from SEQ ID NOs: 02 to 38 and/or a variant GFA1, chosen from SEQ ID NOs: 39 to 53, glmM from E. coli (UniProt ID P31120), glmU from E. coli (UniProt ID P0ACC7), neuC from C. jejuni (UniProt ID AAK91727.1), neuB from N. meningitidis (UniProt ID E0NCD4) and the lactose permease (lacY) from E. coli (UniProt ID P02920). In a next step, the novel strains are transformed with an expression plasmid comprising constitutive transcriptional units for the N-acylneuraminate cytidylyltransferase (neuA) with SEQ ID NO: 55 from P. multocida and either (i) a beta-galactoside alpha-2,3-sialyltransferase like e.g., PmultST3 from P. multocida (UniProt ID Q9CLP3), a PmultST3-like polypeptide comprising amino acid residues 1 to 268 of UniProt ID Q9CLP3 having beta-galactoside alpha-2,3-sialyltransferase activity, NmeniST3 from N. meningitidis (SEQ ID NO: 56) or PmultST2 from P. multocida subsp. Multocida str. Pm70 (SEQ ID NO: 57), or (ii) a beta-galactoside alpha-2,6-sialyltransferase like e.g., PdST6 from Photobacterium damselae (UniProt ID O66375), a PdST6-like polypeptide comprising amino acid residues 108 to 497 of UniProt ID O66375 having beta-galactoside alpha-2,6-sialyltransferase activity, P-JT-ISH-224-ST6 from Photobacterium sp. JT-ISH-224 (UniProt ID A8QYL1) or a P-JT-ISH-224-ST6-like polypeptide comprising amino acid residues 18 to 514 of UniProt ID A8QYL1 having beta-galactoside alpha-2,6-sialyltransferase activity). The novel strains expressing a polypeptide having alpha-2,3-sialyltransferase activity are evaluated for production of ManNAc, Neu5Ac and 3′ SL when evaluated in a 3-days growth experiment according to the culture conditions provided in Example 14 using appropriate selective medium comprising lactose. The novel strains expressing a polypeptide having alpha-2,6-sialyltransferase activity are evaluated for production of ManNAc, Neu5Ac and 6′ SL when evaluated in a 3-days growth experiment according to the culture conditions provided in Example 14 using appropriate selective medium comprising lactose.

### Example 16. Evaluation of Engineered E. coli Strains for the Production of LNT II, LNT or LNnT

A wild-type E. coli K-12 MG1655 strain is modified with genomic knockouts of the E. coli genes LacZ and nagB together with a genomic knock-in of a constitutive transcriptional unit for an GFA1 ortholog, chosen from SEQ ID NOs: 02 to 38 and/or a variant GFA1, chosen from SEQ ID NOs: 39 to 53, and lgtA from N. meningitidis with SEQ ID NO: 54. The novel strains are evaluated for production of LNT II when evaluated in a 3-days growth experiment according to the culture conditions provided in Example 14 using appropriate selective medium comprising lactose.

In a next step for LNT or LNnT production, the LNT II producing E. coli strains are further modified with constitutive transcriptional units for either the N-acetylglucosamine beta-1,3-galactosyltransferase (wbgO) from E. coli O55:H7 (UniProt ID D3QY14) or the N-acetylglucosamine beta-1,4-galactosyltransferase (lgtB) from N. meningitidis (UniProt ID Q51116), respectively. The novel strains expressing wbgO are evaluated for production of LNT II and LNT, when evaluated in a 3-days growth experiment according to the culture conditions provided in Example 14 using appropriate selective medium comprising lactose. The novel strains expressing lgtB are evaluated for production of LNT II and LNnT, when evaluated in a 3-days growth experiment according to the culture conditions provided in Example 14 using appropriate selective medium comprising lactose.

### Example 17. Evaluation of Engineered E. coli Strains for the Production of an Oligosaccharide Mixture Comprising LNT II, Sialylated LNT II, LNT, 3′SL and LSTa

A wild-type E. coli K-12 MG1655 strain is modified with genomic knockouts of the E. coli genes nagA, nagB, lacY, lacZ, nanA, nanE and nanK together with genomic knock-ins of constitutive transcriptional units for an GFA1 ortholog, chosen from SEQ ID NOs: 02 to 38 and/or a variant GFA1, chosen from SEQ ID NOs: 39 to 53, glmM from E. coli (UniProt ID P31120), glmU from E. coli (UniProt ID P0ACC7), neuC from C. jejuni (UniProt ID AAK91727.1), neuB from N. meningitidis (UniProt ID E0NCD4), the lactose permease (lacY) from E. coli (UniProt ID P02920), the beta-1,3-N-acetylglucosaminyltransferase lgtA from N. meningitidis with SEQ ID NO: 54 and the N-acetylglucosamine beta-1,3-galactosyltransferase wbgO from E. coli O55:H7 (UniProt ID D3QY14). In a next step, the novel strains are transformed with an expression plasmid comprising constitutive transcriptional units for neuA with SEQ ID NO: 55 from P. multocida and a beta-galactoside alpha-2,3-sialyltransferase like e.g., PmultST3 from P. multocida (UniProt ID Q9CLP3), a PmultST3-like polypeptide comprising amino acid residues 1 to 268 of UniProt ID Q9CLP3 having beta-galactoside alpha-2,3-sialyltransferase activity or NmeniST3 from N. meningitidis (SEQ ID NO: 56). The novel strains are evaluated for the production of an oligosaccharide mixture comprising LNT II, 3′-sialylated LNT II (Neu5Ac-a2,3-GlcNAc-b1,3-Gal-b1,4-Glc), LNT, 3′SL and LSTa (Neu5Ac-a2,3-Gal-b 1,3-GlcNAc-b 1,3-Gal-b 1,4-Glc) when evaluated in a 3-days growth experiment according to the culture conditions provided in Example 14 using appropriate selective medium comprising lactose.

### Example 18. Evaluation of Engineered E. coli Strains for the Production of an Oligosaccharide Mixture Comprising LNT II, Sialylated LNT II, LNnT, 6′ SL and LSTc

A wild-type E. coli K-12 MG1655 strain is modified with genomic knockouts of the E. coli genes nagA, nagB, lacY, lacZ, nanA, nanE and nanK together with genomic knock-ins of constitutive transcriptional units for an GFA1 ortholog, chosen from SEQ ID NOs: 02 to 38 and/or a variant GFA1, chosen from SEQ ID NOs: 39 to 53, glmM from E. coli (UniProt ID P31120), glmU from E. coli (UniProt ID P0ACC7), neuC from C. jejuni (UniProt ID AAK91727.1), neuB from N. meningitidis (UniProt ID E0NCD4), the lactose permease (lacY) from E. coli (UniProt ID P02920), the beta-1,3-N-acetylglucosaminyltransferase lgtA from N. meningitidis with SEQ ID NO: 54 and the N-acetylglucosamine beta-1,4-galactosyltransferase lgtB from N. meningitidis (UniProt ID Q51116). In a next step, the novel strains are transformed with an expression plasmid comprising constitutive transcriptional units for the N-acylneuraminate cytidylyltransferase (neuA) with SEQ ID NO: 55 from P. multocida and a beta-galactoside alpha-2,6-sialyltransferase like e.g., PdST6 from Photobacterium damselae (UniProt ID O66375), a PdST6-like polypeptide comprising amino acid residues 108 to 497 of UniProt ID O66375 having beta-galactoside alpha-2,6-sialyltransferase activity, P-JT-ISH-224-ST6 from Photobacterium sp. JT-ISH-224 (UniProt ID A8QYL1) or a P-JT-ISH-224-ST6-like polypeptide comprising amino acid residues 18 to 514 of UniProt ID A8QYL1 having beta-galactoside alpha-2,6-sialyltransferase activity. The novel strains are evaluated for production of an oligosaccharide mixture comprising LNT II, 6′-sialylated LNT II (Neu5Ac-a2,6-[GlcNAc-b1,3]-Gal-b1,4-Glc), LNnT, 6′ SL and LSTc (Neu5Ac-a2,6-Gal-b1,4-GlcNAc-b1,3-Gal-b1,4-Glc) when evaluated in a 3-days growth experiment according to the culture conditions provided in Example 14 using appropriate selective medium comprising lactose.

### Example 19. Evaluation of Engineered E. coli Strains for the Production of an Oligosaccharide Mixture Comprising LNT II, Sialylated LNT II, LNnT, 3′ SL and LSTd

A wild-type E. coli K-12 MG1655 strain is modified with genomic knockouts of the E. coli genes nagA, nagB, lacY, lacZ, nanA, nanE and nanK together with genomic knock-ins of constitutive transcriptional units for an GFA1 ortholog, chosen from SEQ ID NOs: 02 to 38 and/or a variant GFA1, chosen from SEQ ID NOs: 39 to 53, glmM from E. coli (UniProt ID P31120), glmU from E. coli (UniProt ID P0ACC7), neuC from C. jejuni (UniProt ID AAK91727.1), neuB from N. meningitidis (UniProt ID E0NCD4), the lactose permease (lacY) from E. coli (UniProt ID P02920), the beta-1,3-N-acetylglucosaminyltransferase lgtA from N. meningitidis with SEQ ID NO: 54 and the N-acetylglucosamine beta-1,4-galactosyltransferase lgtB from N. meningitidis (UniProt ID Q51116). In a next step, the novel strains are transformed with an expression plasmid comprising constitutive transcriptional units for the N-acylneuraminate cytidylyltransferase (neuA) with SEQ ID NO: 55 from P. multocida and a beta-galactoside alpha-2,3-sialyltransferase like e.g., PmultST3 from P. multocida (UniProt ID Q9CLP3), a PmultST3-like polypeptide comprising amino acid residues 1 to 268 of UniProt ID Q9CLP3 having beta-galactoside alpha-2,3-sialyltransferase activity or NmeniST3 from N. meningitidis (SEQ ID NO: 56). The novel strains are evaluated for production of an oligosaccharide mixture comprising LNT II, 3′-sialylated LNT II (Neu5Ac-a2,3-GlcNAc-b1,3-Gal-b1,4-Glc), LNnT, 3′ SL and LSTd (Neu5Ac-a2,3-Gal-b1,4-GlcNAc-b1,3-Gal-b1,4-Glc) when evaluated in a 3-days growth experiment according to the culture conditions provided in Example 14 using appropriate selective medium comprising lactose.

### Example 20. Materials and Methods Bacillus subtilis

Media

Two media are used to cultivate B. subtilis: i.e., a rich Luria Broth (LB) and a minimal medium for shake flask cultures. The LB medium comprised 1% tryptone peptone (Difco), 0.5% yeast extract (Difco) and 0.5% sodium chloride (VWR). Luria Broth agar (LBA) plates comprised the LB media, with 12 g/L agar (Difco) added. The minimal medium contained 2.00 g/L (NH₄)₂SO₄, 7.5 g/L KH₂PO₄, 17.5 g/L K₂HPO₄, 1.25 g/L Na-citrate, 0.25 g/L MgSO₄·7H₂O, 0.05 g/L tryptophan, from 10 up to 30 g/L glucose (or another carbon source including but not limited to fructose, maltose, sucrose, glycerol and maltotriose), 10 mL/L trace element mix and 10 mL/L Fe-citrate solution. The medium was set to a pH of 7.0 with 1 M KOH. Depending on the experiment lactose is added as a precursor. The trace element mix comprised 0.735 g/L CaCl₂·2H₂O, 0.1 g/L MnCl₂·2H₂O, 0.033 g/L CuCl₂·2H₂O, 0.06 g/L CoCl₂·6H₂O, 0.17 g/L ZnCl₂, 0.0311 g/L H₃BO₄, 0.4 g/L Na₂EDTA·2H₂O and 0.06 g/L Na₂MoO₄. The Fe-citrate solution contained 0.135 g/L FeCl₃·6H₂O, 1 g/L Na-citrate (Hoch 1973 PMC1212887).

Complex medium, e.g., LB, was sterilized by autoclaving (121° C., 21 min) and minimal medium by filtration (0.22 μm Sartorius). When necessary, the medium was made selective by adding an antibiotic (e.g., zeocin (20 mg/L)).

Strains, Plasmids and Mutations

Bacillus subtilis 168 is used as available at the Bacillus Genetic Stock Center (Ohio, USA).

Plasmids for gene deletion via Cre/lox are constructed as described by Yan et al. (Appl & Environm microbial, September 2008, p 5556-5562). Gene disruption is done via homologous recombination with linear DNA and transformation via the electroporation as described by Xue et al. (J. Microb. Meth. 34 (1999) 183-191). The method of gene knockouts is described by Liu et al. (Metab. Engine. 24 (2014) 61-69). This method uses 1000 bp homologies up- and downstream of the target gene.

Integrative vectors as described by Popp et al. (Sci. Rep., 2017, 7, 15158) are used as expression vector and could be further used for genomic integrations if necessary. A suitable promoter for expression can be derived from the part repository (iGem): sequence id: Bba_K143012, Bba_K823000, Bba_K823002 or Bba_K823003. Cloning can be performed using Gibson Assembly, Golden Gate assembly, Cliva assembly, LCR or restriction ligation.

In an example for sialic acid (Neu5Ac) production, the engineered strain was derived from B. subtilis comprising knockouts of the B. subtilis nagA, nagB, glmS and gamA genes and genomic knock-ins of constitutive transcriptional units containing one or more variant GFA1 polypeptides chosen from the list comprising SEQ ID NOs: 39 to 53 and/or GFA1 orthologs chosen from the list comprising SEQ ID NOs: 02 to 38, a phosphoglucosamine mutase like e.g., glmM from E. coli (UniProt ID P31120), an N-acetylglucosamine-1-phosphate uridyltransferase/glucosamine-1-phosphate acetyltransferase like e.g., glmU from E. coli (UniProt ID P0ACC7), an UDP-N-acetylglucosamine 2-epimerase like e.g., neuC from C. jejuni (UniProt ID AAK91727.1) and an N-acetylneuraminate synthase like e.g., neuB from E. coli (UniProt ID Q46675).

In an example for sialylated oligosaccharide production, the sialic acid production strains further need to express an N-acylneuraminate cytidylyltransferase like e.g., neuA from P. multocida with SEQ ID NO: 55, and (i) a beta-galactoside alpha-2,3-sialyltransferase like e.g., PmultST3 from P. multocida (UniProt ID Q9CLP3), a PmultST3-like polypeptide comprising amino acid residues 1 to 268 of UniProt ID Q9CLP3 having beta-galactoside alpha-2,3-sialyltransferase activity, NmeniST3 from N. meningitidis (SEQ ID NO: 56) or PmultST2 from P. multocida subsp. Multocida str. Pm70 (SEQ ID NO: 57), (ii) a beta-galactoside alpha-2,6-sialyltransferase like e.g., PdST6 from Photobacterium damselae (UniProt ID O66375), a PdST6-like polypeptide comprising amino acid residues 108 to 497 of UniProt ID O66375 having beta-galactoside alpha-2,6-sialyltransferase activity, an alpha-2,6-sialyltransferase from Photobacterium sp. JT-ISH-224 (UniProt ID A8QYL1) or a polypeptide comprising amino acid residues 18 to 514 of UniProt ID A8QYL1 having beta-galactoside alpha-2,6-sialyltransferase activity, and/or (iii) an alpha-2,8-sialyltransferase like e.g., from M. musculus (UniProt ID Q64689). Constitutive transcriptional units of the N-acylneuraminate cytidylyltransferase and the sialyltransferases can be delivered to the engineered strain either via genomic knock-in or via expression plasmids. If the engineered strains producing sialic acid and CMP-sialic acid were intended to make sialylated lactose structures, the strains were additionally modified with a genomic knock-in of a constitutive transcriptional unit for a lactose permease like e.g., the E. coli LacY (UniProt ID P02920).

In an example for LNT II production, the engineered strain was derived from B. subtilis comprising knockouts of the B. subtilis nagB, glmS and gamA genes and genomic knock-ins of constitutive transcriptional units containing one or more variant GFA1 polypeptides chosen from the list comprising SEQ ID NOs: 39 to 53 and/or GFA1 orthologs chosen from the list comprising SEQ ID NOs: 02 to 38, a galactoside beta-1,3-N-acetylglucosaminyltransferase like e.g., lgtA with SEQ ID NO: 54 from N. meningitidis and a lactose permease like e.g., LacY from E. coli (UniProt ID P02920). For LNT or LNnT production, the LNT II producing strain was further transformed with constitutive transcriptional units for either an N-acetylglucosamine beta-1,3-galactosyltransferase like e.g., wbgO from E. coli O55:H7 (UniProt ID D3QY14) or an N-acetylglucosamine beta-1,4-galactosyltransferase like e.g., lgtB from N. meningitidis (UniProt ID Q51116), respectively.

Heterologous and Homologous Expression

Genes that needed to be expressed, be it from a plasmid or from the genome were synthetically synthetized with one of the following companies: DNA2.0, Gen9, Twist Biosciences or IDT.

Expression could be further facilitated by optimizing the codon usage to the codon usage of the expression host. Genes were optimized using the tools of the supplier.

Cultivation Conditions

A preculture of 96-well microtiter plate experiments was started from a cryovial or a single colony from an LB plate, in 150 μL LB and was incubated overnight at 37° C. on an orbital shaker at 800 rpm. This culture was used as inoculum for a 96-well square microtiter plate, with 400 μL minimal medium by diluting 400×. Each strain was grown in multiple wells of the 96-well plate as biological replicates. These final 96-well culture plates were then incubated at 37° C. on an orbital shaker at 800 rpm for 72h, or shorter, or longer. At the end of the cultivation experiment samples were taken from each well to measure the supernatant concentration (extracellular sugar concentrations, after 5 min. spinning down the cells), or by boiling the culture broth for 15 min at 90° C. or for 60 min at 60° C. before spinning down the cells (=whole broth concentration, intra- and extracellular sugar concentrations, as defined herein).

Also, a dilution of the cultures was made to measure the optical density at 600 nm. The cell performance index or CPI was determined by dividing the oligosaccharide concentrations by the biomass, in relative percentages compared to a reference strain. The biomass is empirically determined to be approximately ⅓rd of the optical density measured at 600 nm.

Optical Density, pH and Analytical Analysis

The determination of the optical density and the pH of the bacterial cultures as well as the analytical analysis were performed as described in Example 1.

### Example 21. Evaluation of Engineered B. subtilis Strains for the Production of ManNAc and Neu5Ac and Either 3′-Sialyllactose (3′SL) or 6′-Sialyllactose (6′SL)

A wild-type B. subtilis strain is first modified with genomic knockouts of the B. subtilis genes nagA, nagB, glmS and gamA together with genomic knock-ins of constitutive transcriptional units for an GFA1 ortholog chosen from SEQ ID NOs: 02 to 38 and/or a variant GFA1 chosen from SEQ ID NOs: 39 to 53, glmM from E. coli (UniProt ID P31120), glmU from E. coli (UniProt ID P0ACC7), neuC from C. jejuni (UniProt ID AAK91727.1), neuB from E. coli (UniProt ID Q46675) and LacY from E. coli (UniProt ID P02920). In a next step, the novel strains are transformed with an expression plasmid comprising constitutive transcriptional units for neuA with SEQ ID NO: 55 from P. multocida and either (i) a beta-galactoside alpha-2,3-sialyltransferase like e.g., PmultST3 from P. multocida (UniProt ID Q9CLP3), a PmultST3-like polypeptide comprising amino acid residues 1 to 268 of UniProt ID Q9CLP3 having beta-galactoside alpha-2,3-sialyltransferase activity or NmeniST3 from N. meningitidis (SEQ ID NO: 56) or (ii) a beta-galactoside alpha-2,6-sialyltransferase like e.g., PdST6 from Photobacterium damselae (UniProt ID O66375), a PdST6-like polypeptide comprising amino acid residues 108 to 497 of UniProt ID O66375 having beta-galactoside alpha-2,6-sialyltransferase activity, an alpha-2,6-sialyltransferase from Photobacterium sp. JT-ISH-224 (UniProt ID A8QYL1) or a polypeptide comprising amino acid residues 18 to 514 of UniProt ID A8QYL1 having beta-galactoside alpha-2,6-sialyltransferase activity. The novel strains expressing a polypeptide having alpha-2,3-sialyltransferase activity are evaluated for production of ManNAc, Neu5Ac and 3′SL when evaluated in a 3-days growth experiment according to the culture conditions provided in Example 20 using appropriate selective medium comprising lactose. The novel strains expressing a polypeptide having alpha-2,6-sialyltransferase activity are evaluated for production of ManNAc, Neu5Ac and 6′SL when evaluated in a 3-days growth experiment according to the culture conditions provided in Example 20 using appropriate selective medium comprising lactose.

### Example 22. Evaluation of Engineered B. subtilis Strains for the Production of LNT II, LNT or LNnT

A wild-type B. subtilis strain is first modified with genomic knockouts of the B. subtilis genes nagB, glmS and gamA together with genomic knock-ins of constitutive transcriptional units for an GFA1 ortholog chosen from SEQ ID NOs: 02 to 38 and/or a variant GFA1 chosen from SEQ ID NOs: 39 to 53, the lactose permease LacY from E. coli (UniProt ID P02920) and lgtA from N. meningitidis with SEQ ID NO: 54. The novel strains are evaluated for the production of LNT II when evaluated in a 3-days growth experiment according to the culture conditions provided in Example 20 using appropriate selective medium comprising lactose.

In a next step for LNT or LNnT production, the LNT II producing B. subtilis strains are further modified with constitutive transcriptional units for either wbgO from E. coli O55:H7 (UniProt ID D3QY14) or lgtB from N. meningitidis (UniProt ID Q51116), respectively. The novel strains expressing wbgO are evaluated for the production of LNT II and LNT, when evaluated in a 3-days growth experiment according to the culture conditions provided in Example 20 using appropriate selective medium comprising lactose. The novel strains expressing lgtB are evaluated for the production of LNT II and LNnT, when evaluated in a 3-days growth experiment according to the culture conditions provided in Example 20 using appropriate selective medium comprising lactose.

### Example 23. Evaluation of Engineered B. subtilis Strains for the Production of an Oligosaccharide Mixture Comprising LNT II, Sialylated LNT II, LNnT, 6′ SL and LSTc

The engineered B. subtilis strains producing LNnT as described in Example 22 are further transformed with an expression plasmid comprising constitutive transcriptional units for the N-acylneuraminate cytidylyltransferase (neuA) from P. multocida (SEQ ID NO: 55) and a beta-galactoside alpha-2,6-sialyltransferase like e.g., PdST6 from Photobacterium damselae (UniProt ID O66375), a PdST6-like polypeptide comprising amino acid residues 108 to 497 of UniProt ID O66375 having beta-galactoside alpha-2,6-sialyltransferase activity, P-JT-ISH-224-ST6 from Photobacterium sp. JT-ISH-224 (UniProt ID A8QYL1) or a P-JT-ISH-224-ST6-like polypeptide comprising amino acid residues 18 to 514 of UniProt ID A8QYL1 having beta-galactoside alpha-2,6-sialyltransferase activity. The novel strains are evaluated for production of an oligosaccharide mixture comprising LNT II, 6′-sialylated LNT II (Neu5Ac-a2,6-[GlcNAc-b1,3]-Gal-b1,4-Glc), LNnT, 6′ SL and LSTc (Neu5Ac-a2,6-Gal-b1,4-GlcNAc-b1,3-Gal-b1,4-Glc) when evaluated in a 3-days growth experiment according to the culture conditions provided in Example 20 using appropriate selective medium comprising lactose.

### Example 24. Materials and Methods Corynebacterium glutamicum

Media

Two different media are used to cultivate C. glutamicum: i.e., a rich tryptone-yeast extract (TY) medium and a minimal medium. The TY medium comprised 1.6% tryptone (Difco), 1% yeast extract (Difco) and 0.5% sodium chloride (VWR). TY agar (TYA) plates comprised the TY media, with 12 g/L agar (Difco) added. The minimal medium for the shake flask experiments contained 20 g/L (NH₄)₂SO₄, 5 g/L urea, 1 g/L KH₂PO₄, 1 g/L K₂HPO₄, 0.25 g/L MgSO₄·7H₂O, 42 g/L MOPS, from 10 up to 30 g/L glucose (or another carbon source including but not limited to fructose, maltose, sucrose, glycerol and maltotriose) and 1 mL/L trace element mix. Depending on the experiment lactose is added as a precursor. The trace element mix comprised 10 g/L CaCl₂), 10 g/L FeSO₄·7H₂O, 10 g/L MnSO₄·H₂O, 1 g/L ZnSO₄·7H₂O, 0.2 g/L CuSO₄, 0.02 g/L NiCl₂·6H₂O, 0.2 g/L biotin (pH 7.0) and 0.03 g/L protocatechuic acid.

Complex medium, e.g., TY, was sterilized by autoclaving (121° C., 21 min) and minimal medium by filtration (0.22 μm Sartorius). When necessary, the medium was made selective by adding an antibiotic (e.g., kanamycin, ampicillin).

Strains and Mutations

Corynebacterium glutamicum ATCC 13032 was used as available at the American Type Culture Collection.

Integrative plasmid vectors based on the Cre/loxP technique as described by Suzuki et al. (Appl. Microbiol. Biotechnol., 2005 April, 67(2):225-33) and temperature-sensitive shuttle vectors as described by Okibe et al. (J. Microbiol. Meth. 85, 2011, 155-163) are constructed for gene deletions, mutations and insertions. Suitable promoters for (heterologous) gene expression can be derived from Yim et al. (Biotechnol. Bioeng., 2013 November, 110(11):2959-69). Cloning can be performed using Gibson Assembly, Golden Gate assembly, Cliva assembly, LCR or restriction ligation.

For Neu5Ac production, the engineered strain was derived from C. glutamicum comprising knockouts of the C. glutamicum ldh, cgl2645, nagB, glmS and nanA genes and genomic knock-ins of constitutive transcriptional units containing one or more variant GFA1 polypeptides chosen from the list comprising SEQ ID NOs: 39 to 53 and/or GFA1 orthologs chosen from the list comprising SEQ ID NOs: 02 to 38, a phosphoglucosamine mutase like e.g., glmM from E. coli (UniProt ID P31120), an N-acetylglucosamine-1-phosphate uridyltransferase/glucosamine-1-phosphate acetyltransferase like e.g., glmU from E. coli (UniProt ID P0ACC7), an UDP-N-acetylglucosamine 2-epimerase like e.g., neuC from C. jejuni (UniProt ID AAK91727.1) and an N-acetylneuraminate synthase like e.g., neuB from E. coli (UniProt ID Q46675).

In an example for sialylated oligosaccharide production, the sialic acid production strains further need to express an N-acylneuraminate cytidylyltransferase like e.g., neuA from P. multocida with SEQ ID NO: 55, and (i) a beta-galactoside alpha-2,3-sialyltransferase like e.g., PmultST3 from P. multocida (UniProt ID Q9CLP3), a PmultST3-like polypeptide comprising amino acid residues 1 to 268 of UniProt ID Q9CLP3 having beta-galactoside alpha-2,3-sialyltransferase activity, NmeniST3 from N. meningitidis (SEQ ID NO: 56) or PmultST2 from P. multocida subsp. Multocida str. Pm70 (SEQ ID NO: 57), (ii) a beta-galactoside alpha-2,6-sialyltransferase like e.g., PdST6 from Photobacterium damselae (UniProt ID O66375), a PdST6-like polypeptide comprising amino acid residues 108 to 497 of UniProt ID O66375 having beta-galactoside alpha-2,6-sialyltransferase activity, an alpha-2,6-sialyltransferase from Photobacterium sp. JT-ISH-224 (UniProt ID A8QYL1) or a polypeptide comprising amino acid residues 18 to 514 of UniProt ID A8QYL1 having beta-galactoside alpha-2,6-sialyltransferase activity, and/or (iii) an alpha-2,8-sialyltransferase like e.g., from M. musculus (UniProt ID Q64689). Constitutive transcriptional units of the N-acylneuraminate cytidylyltransferase and the sialyltransferases can be delivered to the engineered strain either via genomic knock-in or via expression plasmids. If the engineered strains producing sialic acid and CMP-sialic acid were intended to make sialylated lactose structures, the strains were additionally modified with a genomic knock-in of a constitutive transcriptional unit for a lactose permease like e.g., the E. coli LacY (UniProt ID P02920).

In an example for LNT II production, the engineered strain was derived from C. glutamicum comprising knockouts of the C. glutamicum ldh, cgl2645, nagB and glmS genes and genomic knock-ins of constitutive transcriptional units containing one or more variant GFA1 polypeptides chosen from the list comprising SEQ ID NOs: 39 to 53 and/or GFA1 orthologs chosen from the list comprising SEQ ID NOs: 02 to 38, a galactoside beta-1,3-N-acetylglucosaminyltransferase like e.g., lgtA with SEQ ID NO: 54 from N. meningitidis and a lactose permease like e.g., LacY from E. coli (UniProt ID P02920). In an example for LNT or LNnT production, the LNT II producing strains were further transformed with constitutive transcriptional units for either an N-acetylglucosamine beta-1,3-galactosyltransferase like e.g., wbgO from E. coli O55:H7 (UniProt ID D3QY14) or an N-acetylglucosamine beta-1,4-galactosyltransferase like e.g., lgtB from N. meningitidis (UniProt ID Q51116), respectively.

Heterologous and Homologous Expression

Genes that needed to be expressed, be it from a plasmid or from the genome were synthetically synthetized with one of the following companies: DNA2.0, Gen9, Twist Biosciences or IDT.

Expression could be further facilitated by optimizing the codon usage to the codon usage of the expression host. Genes were optimized using the tools of the supplier.

Cultivation Conditions

A preculture of 96-well microtiter plate experiments was started from a cryovial or a single colony from a TY plate, in 150 μL TY and was incubated overnight at 37° C. on an orbital shaker at 800 rpm. This culture was used as inoculum for a 96-well square microtiter plate, with 400 μL minimal medium by diluting 400×. Each strain was grown in multiple wells of the 96-well plate as biological replicates. These final 96-well culture plates were then incubated at 37° C. on an orbital shaker at 800 rpm for 72h, or shorter, or longer. At the end of the cultivation experiment samples were taken from each well to measure the supernatant concentration (extracellular sugar concentrations, after 5 min. spinning down the cells), or by boiling the culture broth for 15 min at 60° C. before spinning down the cells (=whole broth concentration, intra- and extracellular sugar concentrations, as defined herein).

Also, a dilution of the cultures was made to measure the optical density at 600 nm. The cell performance index or CPI was determined by dividing the oligosaccharide concentrations, e.g., sialyllactose concentrations, measured in the whole broth by the biomass, in relative percentages compared to the reference strain. The biomass is empirically determined to be approximately ⅓rd of the optical density measured at 600 nm.

Optical Density, pH and Analytical Analysis

The determination of the optical density and the pH of the bacterial cultures as well as the analytical analysis were performed as described in Example 1.

### Example 25. Evaluation of Engineered C. glutamicum Strains for the Production of ManNAc and Neu5Ac and Either 3′-Sialyllactose (3′SL) or 6′-Sialyllactose (6′SL)

A wild-type C. glutamicum strain is first modified with genomic knockouts of the C. glutamicum genes ldh, cgl2645, nagB, glmS and nanA, together with genomic knock-ins of constitutive transcriptional units for an GFA1 ortholog chosen from SEQ ID NOs: 02 to 38 and/or a variant GFA1 chosen from SEQ ID NOs: 39 to 53, glmM from E. coli (UniProt ID P31120), glmU from E. coli (UniProt ID P0ACC7), neuC from C. jejuni (UniProt ID AAK91727.1), neuB from E. coli (UniProt ID Q46675) and LacY from E. coli (UniProt ID P02920). In a next step, the novel strain is transformed with an expression plasmid comprising constitutive transcriptional units for neuA with SEQ ID NO: 55 from P. multocida and either (i) a beta-galactoside alpha-2,3-sialyltransferase like e.g., PmultST3 from P. multocida (UniProt ID Q9CLP3), a PmultST3-like polypeptide comprising amino acid residues 1 to 268 of UniProt ID Q9CLP3 having beta-galactoside alpha-2,3-sialyltransferase activity or NmeniST3 from N. meningitidis (SEQ ID NO: 56) or (ii) a beta-galactoside alpha-2,6-sialyltransferase like e.g., PdST6 from Photobacterium damselae (UniProt ID O66375), a PdST6-like polypeptide comprising amino acid residues 108 to 497 of UniProt ID O66375 having beta-galactoside alpha-2,6-sialyltransferase activity, an alpha-2,6-sialyltransferase from Photobacterium sp. JT-ISH-224 (UniProt ID A8QYL1) or a polypeptide comprising amino acid residues 18 to 514 of UniProt ID A8QYL1 having beta-galactoside alpha-2,6-sialyltransferase activity. The novel strains expressing a polypeptide having alpha-2,3-sialyltransferase activity are evaluated for production of ManNAc, Neu5Ac and 3′ SL when evaluated in a 3-days growth experiment according to the culture conditions provided in Example 24 using appropriate selective medium comprising lactose. The novel strains expressing a polypeptide having alpha-2,6-sialyltransferase activity are evaluated for production of ManNAc, Neu5Ac and 6′ SL when evaluated in a 3-days growth experiment according to the culture conditions provided in Example 24 using appropriate selective medium comprising lactose.

### Example 26. Evaluation of Engineered C. glutamicum Strains for the Production of LNT II, LNT or LNnT

A wild-type C. glutamicum strain is first modified with genomic knockouts of the C. glutamicum genes ldh, cgl2645, nagB and glmS together with genomic knock-ins of constitutive transcriptional units for an GFA1 ortholog chosen from SEQ ID NOs: 02 to 38 and/or a variant GFA1 chosen from SEQ ID NOs: 39 to 53, the lactose permease LacY from E. coli (UniProt ID P02920) and lgtA from N. meningitidis with SEQ ID NO: 54. The novel strains are evaluated for production of LNT II when evaluated in a 3-days growth experiment according to the culture conditions provided in Example 23 using appropriate selective medium comprising lactose.

In a next step for LNT or LNnT production, the LNT II producing C. glutamicum strains are further modified with constitutive transcriptional units for either wbgO from E. coli O55:H7 (UniProt ID D3QY14) or lgtB from N. meningitidis (UniProt ID Q51116), respectively. The novel strains expressing wbgO are evaluated for the production of LNT II and LNT, when evaluated in a 3-days growth experiment according to the culture conditions provided in Example 24 using appropriate selective medium comprising lactose. The novel strains expressing lgtB are evaluated for the production of LNT II and LNnT, when evaluated in a 3-days growth experiment according to the culture conditions provided in Example 24 using appropriate selective medium comprising lactose.

### Example 27. Evaluation of Engineered C. glutamicum Strains for the Production of an Oligosaccharide Mixture Comprising LNT II, Sialylated LNT II, LNT, 3′SL and LSTa

The engineered C. glutamicum strains producing LNT as described in Example 26 are further transformed with an expression plasmid comprising constitutive transcriptional units for neuA from P. multocida (SEQ ID NO: 55) and a beta-galactoside alpha-2,3-sialyltransferase like e.g., PmultST3 from P. multocida (UniProt ID Q9CLP3), a PmultST3-like polypeptide comprising amino acid residues 1 to 268 of UniProt ID Q9CLP3 having beta-galactoside alpha-2,3-sialyltransferase activity or NmeniST3 from N. meningitidis (SEQ ID NO: 56). The novel strains are evaluated for the production of an oligosaccharide mixture comprising LNT II, 3′-sialylated LNT II (Neu5Ac-a2,3-GlcNAc-b1,3-Gal-b1,4-Glc), LNT, 3′SL and LSTa (Neu5Ac-a2,3-Gal-b1,3-GlcNAc-b1,3-Gal-b1,4-Glc) when evaluated in a 3-days growth experiment according to the culture conditions provided in Example 24 using appropriate selective medium comprising lactose.

### Example 28. Materials and Methods Chlamydomonas reinhardtii

Media

C. reinhardtii cells were cultured in Tris-acetate-phosphate (TAP) medium (pH 7.0). The TAP medium uses a 1000× stock Hutner's trace element mix. Hutner's trace element mix comprised 50 g/L Na₂EDTA·H₂O (Titriplex III), 22 g/L ZnSO₄·7H₂O, 11.4 g/L H₃BO₃, 5 g/L MnCl₂·4H₂O, 5 g/L FeSO₄·7H₂O, 1.6 g/L CoCl₂·6H₂O, 1.6 g/L CuSO₄·5H₂O and 1.1 g/L (NH₄)₆MoO₃.

The TAP medium contained 2.42 g/L Tris (tris(hydroxymethyl)aminomethane), 25 mg/L salt stock solution, 0.108 g/L K₂HPO₄, 0.054 g/L KH₂PO₄ and 1.0 mL/L glacial acetic acid. The salt stock solution comprised 15 g/L NH₄Cl, 4 g/L MgSO₄·7H₂O and 2 g/L CaCl₂·2H₂O. As precursor for saccharide synthesis, precursors like e.g., galactose, glucose, fructose, fucose, GlcNAc could be added. Medium was sterilized by autoclaving (121° C., 21 min). For stock cultures on agar slants TAP medium was used containing 1% agar (of purified high strength, 1000 g/cm²).

Strains, Plasmids and Mutations

C. reinhardtii wild-type strains 21 gr (CC-1690, wild-type, mt+), 6145C (CC-1691, wild-type, mt−), CC-125 (137c, wild-type, mt+), CC-124 (137c, wild-type, mt−) as available from Chlamydomonas Resource Center (www.chlamycollection.org), University of Minnesota, U.S.A.

Expression plasmids originated from pSI103, as available from Chlamydomonas Resource Center. Cloning can be performed using Gibson Assembly, Golden Gate assembly, Cliva assembly, LCR or restriction ligation. Suitable promoters for (heterologous) gene expression can be derived from e.g., Scranton et al. (Algal Res. 2016, 15: 135-142). Targeted gene modification (like gene knock-out or gene replacement) can be carried using the Crispr-Cas technology as described e.g., by Jiang et al. (Eukaryotic Cell 2014, 13(11): 1465-1469).

Transformation via electroporation was performed as described by Wang et al. (Biosci. Rep. 2019, 39: BSR2018210). Cells were grown in liquid TAP medium under constant aeration and continuous light with a light intensity of 8000 Lx until the cell density reached 1.0-2.0×10⁷ cells/mL. Then, the cells were inoculated into fresh liquid TAP medium in a concentration of 1.0×10⁶ cells/mL and grown under continuous light for 18-20 h until the cell density reached 4.0×10⁶ cells/mL. Next, cells were collected by centrifugation at 1250 g for 5 min at room temperature, washed and resuspended with pre-chilled liquid TAP medium containing 60 mM sorbitol (Sigma, U.S.A.), and iced for 10 min. Then, 250 μL of cell suspension (corresponding to 5.0×10⁷ cells) were placed into a pre-chilled 0.4 cm electroporation cuvette with 100 ng plasmid DNA (400 ng/mL). Electroporation was performed with 6 pulses of 500 V each having a pulse length of 4 ms and pulse interval time of 100 ms using a BTX ECM830 electroporation apparatus (1575 Ω, 50 μFD). After electroporation, the cuvette was immediately placed on ice for 10 min. Finally, the cell suspension was transferred into a 50 mL conical centrifuge tube containing 10 mL of fresh liquid TAP medium with 60 mM sorbitol for overnight recovery at dim light by slowly shaking. After overnight recovery, cells were recollected and plated with starch embedding method onto selective 1.5% (w/v) agar-TAP plates containing ampicillin (100 mg/L) or chloramphenicol (100 mg/L). Plates were then incubated at 23+−0.5° C. under continuous illumination with a light intensity of 8000 Lx. Cells were analyzed 5-7 days later.

In an example for production of UDP-galactose, C. reinhardtii cells are modified with transcriptional units comprising the gene encoding the galactokinase from Arabidopsis thaliana (KIN, UniProt ID Q9SEE5) and the gene encoding the UDP-sugar pyrophosphorylase (USP) from A. thaliana (UniProt ID Q9C51I).

In an example for enhanced production of UDP-GlcNAc, C. reinhardtii cells are modified with a transcriptional unit comprising a gene encoding a variant GFA1 polypeptide chosen from the list comprising SEQ ID NOs: 39 to 53 and/or a GFA1 ortholog chosen from the list comprising SEQ ID NOs: 02 to 38.

In an example for LNT II production, C. reinhardtii cells are modified with a constitutive transcriptional unit comprising a galactoside beta-1,3-N-acetylglucosaminyltransferase like e.g., lgtA from N. meningitidis (UniProt ID Q9JXQ6). In an example for LNT production, the LNT II producing strain is further modified with a constitutive transcriptional unit comprising an N-acetylglucosamine beta-1,3-galactosyltransferase like e.g., WbgO from E. coli O55:H7 (UniProt ID D3QY14). In an example for LNnT production, the LNT II producing strain is further modified with a constitutive transcriptional unit comprising an N-acetylglucosamine beta-1,4-galactosyltransferase like e.g., lgtB from N. meningitidis (UniProt ID Q51116).

In an example for CMP-sialic acid synthesis, C. reinhardtii cells are modified with constitutive transcriptional units for a UDP-N-acetylglucosamine-2-epimerase/N-acetylmannosamine kinase like e.g., GNE from Homo sapiens (UniProt ID Q9Y223) or a mutant form of the human GNE polypeptide comprising the R263L mutation, an N-acylneuraminate-9-phosphate synthetase like e.g., NANS from Homo sapiens (UniProt ID Q9NR45) and an N-acylneuraminate cytidylyltransferase like e.g., CMAS from Homo sapiens (UniProt ID Q8NFW8). In an example for production of sialylated oligosaccharides, C. reinhardtii cells are modified with a CMP-sialic acid transporter like e.g., CST from Mus musculus (UniProt ID Q61420), and a Golgi-localized sialyltransferase chosen from species like e.g., Homo sapiens, Mus musculus, Rattus norvegicus.

Heterologous and Homologous Expression

Genes that needed to be expressed, be it from a plasmid or from the genome were synthetically synthetized with one of the following companies: DNA2.0, Gen9, Twist Biosciences or IDT.

Expression could be further facilitated by optimizing the codon usage to the codon usage of the expression host. Genes were optimized using the tools of the supplier.

Cultivation Conditions

Cells of C. reinhardtii were cultured in selective TAP-agar plates at 23+/−0.5° C. under 14/10 h light/dark cycles with a light intensity of 8000 Lx. Cells were analyzed after 5 to 7 days of cultivation.

For high-density cultures, cells could be cultivated in closed systems like e.g., vertical or horizontal tube photobioreactors, stirred tank photobioreactors or flat panel photobioreactors as described by Chen et al. (Bioresour. Technol. 2011, 102: 71-81) and Johnson et al. (Biotechnol. Prog. 2018, 34: 811-827).

### Example 29. Production of an Oligosaccharide Mixture Comprising LNT II, Sialylated LNT II, LNnT, 6′ SL and LSTc in Modified C. reinhardtii Cells

C. reinhardtii cells are engineered as described in Example 28 for production of UDP-Gal with genomic knock-ins of constitutive transcriptional units comprising the galactokinase from A. thaliana (KIN, UniProt ID Q9SEE5) and the UDP-sugar pyrophosphorylase (USP) from A. thaliana (UniProt ID Q9C5I1). In a next step, the engineered cells are modified for CMP-sialic acid synthesis with genomic knock-ins of constitutive transcriptional units comprising a variant GFA1 polypeptide chosen from the list comprising SEQ ID NOs: 39 to 53 and/or a GFA1 ortholog chosen from the list comprising SEQ ID NOs: 02 to 38, a mutant form of the UDP-N-acetylglucosamine-2-epimerase/N-acetylmannosamine kinase GNE from Homo sapiens (UniProt ID Q9Y223) differing from the native polypeptide with a R263L mutation, the N-acylneuraminate-9-phosphate synthetase NANS from Homo sapiens (UniProt ID Q9NR45), the N-acylneuraminate cytidylyltransferase CMAS from Homo sapiens (UniProt ID Q8NFW8) and the CMP-sialic acid transporter CST from Mus musculus (UniProt ID Q61420). In a final step, the engineered cells are modified with an expression plasmid comprising constitutive transcriptional units comprising the alpha-2,6-sialyltransferase (UniProt ID P13721) from Rattus norvegicus, the alpha-2,6-sialyltransferase PdST6 from Photobacterium damselae (UniProt ID O66375), the galactoside beta-1,3-N-acetylglucosaminyltransferase lgtA from N. meningitis (UniProt ID Q9JXQ6) and the N-acetylglucosamine beta-1,4-galactosyltransferase lgtB from N. meningitidis (UniProt ID Q51116). The novel strains are evaluated for production of an oligosaccharide mixture comprising LNT II, 6′-sialylated LNT II (Neu5Ac-a2,6-[GlcNAc-b1,3]-Gal-b1,4-Glc), LNnT, 6′SL and LSTc (Neu5Ac-a2,6-Gal-b1,4-GlcNAc-b1,3-Gal-b1,4-Glc) in a cultivation experiment on TAP-agar plates comprising galactose, glucose and GlcNAc as precursors according to the culture conditions provided in Example 28. After 5 days of incubation, the cells are harvested, and the saccharide production is analyzed on UPLC.

### Example 30. Materials and Methods Animal Cells

Isolation of Mesenchymal Stem Cells from Adipose Tissue of Different Mammals

Fresh adipose tissue is obtained from slaughterhouses (e.g., cattle, pigs, sheep, chicken, ducks, catfish, snake, frogs) or liposuction (e.g., in case of humans, after informed consent) and kept in phosphate buffer saline supplemented with antibiotics. Enzymatic digestion of the adipose tissue is performed followed by centrifugation to isolate mesenchymal stem cells. The isolated mesenchymal stem cells are transferred to cell culture flasks and grown under standard growth conditions, e.g., 37° C., 5% CO2. The initial culture medium includes DMEM-F12, RPMI, and Alpha-MEM medium (supplemented with 15% fetal bovine serum), and 1% antibiotics. The culture medium is subsequently replaced with 10% FBS (fetal bovine serum)-supplemented media after the first passage. For example, Ahmad and Shakoori (2013, Stem Cell Regen. Med. 9(2): 29-36), which is incorporated herein by reference in its entirety for all purposes, describes certain variation(s) of the method(s) described herein in this example.

Isolation of Mesenchymal Stem Cells from Milk

This example illustrates isolation of mesenchymal stem cells from milk collected under aseptic conditions from human or any other mammal(s) such as described herein. An equal volume of phosphate buffer saline is added to diluted milk, followed by centrifugation for 20 min. The cell pellet is washed thrice with phosphate buffer saline and cells are seeded in cell culture flasks in DMEM-F12, RPMI, and Alpha-MEM medium supplemented with 10% fetal bovine serum and 1% antibiotics under standard culture conditions. For example, Hassiotou et al. (2012, Stem Cells. 30(10): 2164-2174), which is incorporated herein by reference in its entirety for all purposes, describes certain variation(s) of the method(s) described herein in this example.

Differentiation of Stem Cells Using 2D and 3D Culture Systems

The isolated mesenchymal cells can be differentiated into mammary-like epithelial and luminal cells in 2D and 3D culture systems. See, for example, Huynh et al. 1991. Exp Cell Res. 197(2): 191-199; Gibson et al. 1991, In Vitro Cell Dev Biol Anim. 27(7): 585-594; Blatchford et al. 1999; Animal Cell Technology’: Basic & Applied Aspects, Springer, Dordrecht. 141-145; Williams et al. 2009, Breast Cancer Res 11(3): 26-43; and Arevalo et al. 2015, Am J Physiol Cell Physiol. 310(5): C348-C356; each of which is incorporated herein by reference in their entireties for all purposes.

For 2D culture, the isolated cells were initially seeded in culture plates in growth media supplemented with 10 ng/mL epithelial growth factor and 5 pg/mL insulin. At confluence, cells were fed with growth medium supplemented with 2% fetal bovine serum, 1% penicillin-streptomycin (100 U/mL penicillin, 100 ug/mL streptomycin), and 5 pg/mL insulin for 48h. To induce differentiation, the cells were fed with complete growth medium containing 5 pg/mL insulin, 1 pg/mL hydrocortisone, 0.65 ng/mL triiodothyronine, 100 nM dexamethasone, and 1 pg/mL prolactin. After 24h, serum is removed from the complete induction medium.

For 3D culture, the isolated cells were trypsinized and cultured in Matrigel, hyaluronic acid, or ultra-low attachment surface culture plates for six days and induced to differentiate and lactate by adding growth media supplemented with 10 ng/mL epithelial growth factor and 5 pg/mL insulin. At confluence, cells were fed with growth medium supplemented with 2% fetal bovine serum, 1% penicillin-streptomycin (100 U/mL penicillin, 100 ug/mL streptomycin), and 5 pg/mL insulin for 48h. To induce differentiation, the cells were fed with complete growth medium containing 5 pg/mL insulin, 1 pg/mL hydrocortisone, 0.65 ng/mL triiodothyronine, 100 nM dexamethasone, and 1 pg/mL prolactin. After 24h, serum is removed from the complete induction medium.

Method of Making Mammary-Like Cells

Mammalian cells are brought to induced pluripotency by reprogramming with viral vectors encoding for Oct4, Sox2, Klf4, and c-Myc. The resultant reprogrammed cells are then cultured in Mammocult media (available from Stem Cell Technologies), or mammary cell enrichment media (DMEM, 3% FBS, estrogen, progesterone, heparin, hydrocortisone, insulin, EGF) to make them mammary-like, from which expression of select milk components can be induced. Alternatively, epigenetic remodeling is performed using remodeling systems such as CRISPR/Cas9, to activate select genes of interest, such as casein, a-lactalbumin to be constitutively on, to allow for the expression of their respective proteins, and/or to down-regulate and/or knock-out select endogenous genes as described e.g., in WO21067641, which is incorporated herein by reference in its entirety for all purposes.

Cultivation

Completed growth media includes high glucose DMEM/F12, 10% FBS, 1% NEAA, 1% pen/strep, 1% ITS-X, 1% F-Glu, 10 ng/mL EGF, and 5 pg/mL hydrocortisone. Completed lactation media includes high glucose DMEM/F12, 1% NEAA, 1% pen/strep, 1% ITS-X, 1% F-Glu, 10 ng/mL EGF, 5 pg/mL hydrocortisone, and 1 pg/mL prolactin (5 ug/mL in Hyunh 1991). Cells are seeded at a density of 20,000 cells/cm² onto collagen coated flasks in completed growth media and left to adhere and expand for 48 hours in completed growth media, after which the media is switched out for completed lactation media. Upon exposure to the lactation media, the cells start to differentiate and stop growing. Within about a week, the cells start secreting lactation product(s) such as milk lipids, lactose, casein and whey into the media. A desired concentration of the lactation media can be achieved by concentration or dilution by ultrafiltration. A desired salt balance of the lactation media can be achieved by dialysis, for example, to remove unwanted metabolic products from the media. Hormones and other growth factors used can be selectively extracted by resin purification, for example, the use of nickel resins to remove His-tagged growth factors, to further reduce the levels of contaminants in the lactated product.

### Example 31. Production of an Oligosaccharide Mixture Comprising Sialylated Oligosaccharides in a Non-Mammary Adult Stem Cell

Isolated mesenchymal cells and re-programmed into mammary-like cells as described in Example 30 are modified via CRISPR-CAS to express a variant GFA1 polypeptide chosen from the list comprising SEQ ID NOs: 39 to 53 and/or a GFA1 ortholog chosen from the list comprising SEQ ID NOs: 02 to 38, the GlcN6P synthase from Homo sapiens (UniProt ID Q06210), the glucosamine 6-phosphate N-acetyltransferase from Homo sapiens (UniProt ID Q96EK6), the phosphoacetylglucosamine mutase from Homo sapiens (UniProt ID O95394), the UDP-N-acetylhexosamine pyrophosphorylase (UniProt ID Q16222), the galactoside beta-1,3-N-acetylglucosaminyltransferase LgtA from N. meningitidis (UniProt ID Q9JXQ6), the N-acetylglucosamine beta-1,3-galactosyltransferase WbgO from E. coli O55:H7 (UniProt ID D3QY14), the N-acylneuraminate cytidylyltransferases from Mus musculus (UniProt ID Q99KK2), the CMP-N-acetylneuraminate-beta-1,4-galactoside alpha-2,3-sialyltransferase ST3GAL3 from Homo sapiens (UniProt ID Q11203) and the alpha-2,6-sialyltransferase (UniProt ID P13721) from Rattus norvegicus. Cells are seeded at a density of 20,000 cells/cm 2 onto collagen coated flasks in completed growth media and left to adhere and expand for 48 hours in completed growth media, after which the media is switched out for completed lactation media for about 7 days. After cultivation as described in Example 30, cells are subjected to UPLC to analyze for production of an oligosaccharide mixture comprising LNT, 3′ SL, 6′ SL, sialylated LNT II and LSTa.

1.-26. (canceled)

27. A metabolically engineered cell for producing a glycosylated product comprising a disaccharide or oligosaccharide that comprises at least two different monosaccharide subunits selected from the group consisting of a mammalian milk di- or oligosaccharide, a human milk di- or oligosaccharide, 0-antigen, enterobacterial common antigen (ECA), the oligosaccharide repeats present in capsular polysaccharides, peptidoglycan (PG), and antigens of the human ABO blood group system, wherein the cell:
(i) is capable of expressing a variant yeast or fungal glutamine:fructose-6-phosphate aminotransferase that differs from SEQ ID NO:1 by a V12L, a Q96H, a Q157R and/or an E343V mutation and having glutamine:fructose-6-phosphate aminotransferase activity, and
(ii) is capable of synthesizing UDP-N-acetylglucosamine (UDP-GlcNAc), and
(iii) is capable of expressing a glycosyltransferase,
wherein the cell utilizes UDP-GlcNAc to produce the glycosylated product.

28. The cell of claim 27, wherein the cell is modified with at least one gene expression module comprising the variant yeast or fungal glutamine-fructose-6-phosphate aminotransferase.

29. The cell of claim 27, wherein the variant yeast or fungal glutamine-fructose-6-phosphate aminotransferase is a protein that:
(i) comprises the polypeptide sequence of any one of SEQ ID NOs: 39, 40, 41, 42, 43, 44, 45, 46, 47, 48, 49, 50, 51, 52 or 53, or
(ii) comprises an amino acid sequence that is at least 80.0% sequence identical over a stretch of at least 200 amino acid residues to the amino acid sequence of any one of SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52 or 53, respectively.

30. The cell of claim 27, wherein at least one of the monosaccharide subunits is selected from the group consisting of N-acetylglucosamine, N-acetylmannosamine, N-acetylgalactosamine, 2-acetamido-2,6-dideoxy-L-arabino-4-hexulose, 2-acetamido-2,6-dideoxy-L-lyxo-4-hexulose, N-acetyl-L-rhamnosamine, N-acetyl-D-fucosamine, N-acetyl-L-pneumosamine, N-acetylmuramic acid, N-acetyl-L-quinovosamine, N-acetylneuraminic acid, and N-glycolylneuraminic acid.

31. The cell of claim 27, wherein the cell further synthesizes a nucleotide-sugar selected from the group consisting of UDP-N-acetylgalactosamine (UDP-GalNAc), UDP-N-acetylmannosamine (UDP-ManNAc), UDP-glucose (UDP-Glc), UDP-galactose (UDP-Gal), GDP-mannose (GDP-Man), UDP-glucuronate, UDP-galacturonate, UDP-2-acetamido-2,6-dideoxy-L-arabino-4-hexulose, UDP-2-acetamido-2,6-dideoxy-L-lyxo-4-hexulose, UDP-N-acetyl-L-rhamnosamine (UDP-L-RhaNAc or UDP-2-acetamido-2,6-dideoxy-L-mannose), dTDP-N-acetylfucosamine, UDP-N-acetylfucosamine (UDP-L-FucNAc or UDP-2-acetamido-2,6-dideoxy-L-galactose), UDP-N-acetyl-L-pneumosamine (UDP-L-PneNAC or UDP-2-acetamido-2,6-dideoxy-L-talose), UDP-N-acetylmuramic acid, UDP-N-acetyl-L-quinovosamine (UDP-L-QuiNAc or UDP-2-acetamido-2,6-dideoxy-L-glucose), CMP-sialic acid (C1VIP-Neu5Ac), CMP-N-glycolylneuraminic acid (CMP-Neu5Gc), GDP-fucose (GDP-Fuc), GDP-rhamnose, and UDP-xylose.

32. The cell of claim 27, wherein the glycosyltransferase is selected from the group consisting of fucosyltransferases, sialyltransferases, galactosyltransferases, glucosyltransferases, mannosyltransferases, N-acetylglucosaminyltransferases, N-acetylgalactosaminyltransferases, N-acetylmannosaminyltransferases, xylosyltransferases, glucuronyltransferases, galacturonyltransferases, glucosaminyltransferases, N-glycolylneuraminyltransferases, rhamnosyltransferases, N-acetylrhamnosyltransferases, UDP-4-amino-4,6-dideoxy-N-acetyl-beta-L-altrosamine transaminases, UDP-N-acetylglucosamine enolpyruvyl transferases, and fucosaminyltransferases.

33. The cell of claim 27, wherein the cell further expresses at least one enzyme selected from the group consisting of glucosamine 6-phosphate N-acetyltransferase, phosphoacetylglucosamine mutase, UDP-N-acetylglucosamine pyrophosphorylase, galactoside beta-1,3-N-acetylglucosaminyltransferase, UTP-glucose-1-phosphate uridylyltransferase, UDP-glucose 4-epimerase, N-acetylglucosamine beta-1,3-galactosyltransferase, N-acetylglucosamine beta-1,4-galactosyltransferase, lactose permease, UDP-N-acetylglucosamine 2-epimerase, N-acetylneuraminate synthase, N-acylneuraminate cytidylyltransferase, glucose-6-phosphate isomerase, and UDP-2-acetamido-2,6-dideoxy-L-talose 2-epimerase.

34. The cell of claim 27, wherein the cell utilizes at least one precursor for the synthesis of the glycosylated product.

35. The cell of claim 27, wherein the cell produces at least one precursor for the synthesis of the glycosylated product.

36. The cell of claim 34, wherein the precursor for the synthesis of the glycosylated product is completely converted into the glycosylated product.

37. The cell of claim 27, wherein the cell excretes at least one disaccharide or oligosaccharide out of the cell.

38. The cell of claim 27, wherein the cell is a bacterium, fungus, yeast, a plant cell, an animal cell, or a protozoan cell.

39. A method of producing a glycosylated product comprising a disaccharide or oligosaccharide comprising at least two different monosaccharide subunits selected from the group consisting of a mammalian milk di- or oligosaccharide, a human milk di- or oligosaccharide, O-antigen, enterobacterial common antigen (ECA), the oligosaccharide repeats present in capsular polysaccharides, peptidoglycan (PG), and antigens of the human ABO blood group system by a cell, the method comprising the steps of:
(a) cultivating the cell of claim 27 under conditions permissive to produce the glycosylated product, and
(b) optionally separating the glycosylated product from the cultivation.

40. The method according to claim 39, wherein during cultivation the cell excretes the glycosylated product out of the cell.

41. The method according to claim 39, including a separating step comprising at least one of the following steps: clarification, ultrafiltration, nanofiltration, two-phase partitioning, reverse osmosis, microfiltration, activated charcoal or carbon treatment, treatment with non-ionic surfactants, enzymatic digestion, tangential flow high-performance filtration, tangential flow ultrafiltration, affinity chromatography, ion exchange chromatography, hydrophobic interaction chromatography and/or gel filtration, ligand exchange chromatography, electrodialysis.

42. The method according to claim 39, further comprising purification of the glycosylated product from the cell.

43. The method according to claim 42, wherein the purification comprises at least one of the following steps: use of activated charcoal or carbon, use of charcoal, nanofiltration, ultrafiltration, electrophoresis, enzymatic treatment or ion exchange, temperature adjustment, pH adjustment, pH adjustment with an alkaline or acidic solution, use of alcohols, use of aqueous alcohol mixtures, crystallization, evaporation, precipitation, drying, spray drying, lyophilization, spray freeze drying, freeze spray drying, band drying, belt drying, vacuum band drying, vacuum belt drying, drum drying, roller drying, vacuum drum drying or vacuum roller drying.

44. A vector comprising an isolated nucleic acid molecule encoding a variant yeast or fungal glutamine:fructose-6-phosphate aminotransferase, wherein the variant yeast or fungal glutamine:fructose-6-phosphate aminotransferase is a protein having glutamine:fructose-6-phosphate aminotransferase activity and that differs from SEQ ID NO: 1 by a V12L, a Q96H, a Q157R and/or an E343V mutation and that:
(i) comprises the polypeptide sequence of any one of SEQ ID NOs: 39, 40, 41, 42, 43, 44, 45, 46, 47, 48, 49, 50, 51, 52 or 53 that, or
(ii) comprises an amino acid sequence that is at least 80.0% sequence identical over a stretch of at least 200 amino acid residues to the amino acid sequence of any one of SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52 or 53, respectively.

45. A method of using an isolated nucleic acid molecule encoding a variant yeast or fungal glutamine:fructose-6-phosphate aminotransferase, wherein the variant yeast or fungal glutamine:fructose-6-phosphate aminotransferase is a protein having glutamine:fructose-6-phosphate aminotransferase activity and that differs from SEQ ID NO: 1 by a V12L, a Q96H, a Q157R and/or an E343V mutation and that:
(i) comprises the polypeptide sequence of any one of SEQ ID NOs: 39, 40, 41, 42, 43, 44, 45, 46, 47, 48, 49, 50, 51, 52 or 53 that, or
(ii) comprises an amino acid sequence that is at least 80.0% sequence identical over a stretch of at least 200 amino acid residues to the amino acid sequence of any one of SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52 or 53, respectively,
for producing a glycosylated product that is derived from UDP-GlcNAc and comprises a disaccharide or oligosaccharide that comprises at least two different monosaccharide subunits and that is selected from the group consisting of a mammalian milk di- or oligosaccharide, preferably a human milk di- or oligosaccharide, O-antigen, enterobacterial common antigen (ECA), the oligosaccharide repeats present in capsular polysaccharides, peptidoglycan (PG) and antigens of the human ABO blood group system, the method comprising:
expressing the isolated nucleic acid molecule in a host cell so as to produce the glycosylated product.

46. A method of using the vector of claim 44 for producing a glycosylated product that is derived from UDP-GlcNAc and comprises a disaccharide or oligosaccharide that comprises at least two different monosaccharide subunits selected from the group consisting of a mammalian milk di- or oligosaccharide, a human milk di- or oligosaccharide, O-antigen, enterobacterial common antigen (ECA), the oligosaccharide repeats present in capsular polysaccharides, peptidoglycan (PG), and antigens of the human ABO blood group system, the method comprising:
expressing the vector in a host cell so as to produce the glycosylated product.


SEQUENCE LISTING (57 sequences)

SEQ ID NO: 1 - protein, 717 aa, Saccharomyces cerevisiae S288C
MCGIFGYCNYLVERSRGEIIDTLVDGLQRLEYRGYDSTGIAIDGDEADSTFIYKQIGKVSALKEEITKQNPNRDVTFVSHCGIAHTRWATHGRPEQVNCHPQRSDPEDQFVVVHNGIITNFRELKTLLINKGYKFESDTDTECIAKLYLHLYNTNLQNGHDLDFHELTKLVLLELEGSYGLLCKSCHYPNEVIATRKGSPLLIGVKSEKKLKVDFVDVEFPEENAGQPEIPLKSNNKSFGLGPKKAREFEAGSQNANLLPIAANEFNLRHSQSRAFLSEDGSPTPVEFFVSSDAASVVKHTKKVLFLEDDDLAHIYDGELHIHRSRREVGASMTRSIQTLEMELAQIMKGPYDHFMQKEIYEQPESTFNTMRGRIDYENNKVILGGLKAWLPVVRRARRLIMIACGTSYHSCLATRAIFEELSDIPVSVELASDFLDRKCPVFRDDVCVFVSQSGETADTMLALNYCLERGALTVGIVNSVGSSISRVTHCGVHINAGPEIGVASTKAYTSQYIALVMFALSLSDDRVSKIDRRIEIIQGLKLIPGQIKQVLKLEPRIKKLCATELKDQKSLLLLGRGYQFAAALEGALKIKEISYMHSEGVLAGELKHGVLALVDENLPIIAFGTRDSLFPKVVSSIEQVTARKGHPIIICNENDEVWAQKSKSIDLQTLEVPQTVDCLQGLINIIPLQLMSYWLAVNKGIDVDFPRNLAKSVTVE

SEQ ID NO: 2 - protein, 717 aa, Saccharomyces pastorianus strain CBS 1483
MCGIFGYCNYLVERSRGEIIDTLMDGLQRLEYRGYDSTGIAIDGDEADSTFIYKQIGKVSALKEEITKQNPNRDVTFVSHCGIAHTRWATHGRPEQVNCHPQRSDPEDQFVVVHNGIITNFRELKTLLINKGYKFESDTDTECIAKLYLHLYNTNLQNGHDLDFHELTKLVLLELEGSYGLLCKSCHYPNEVIATRKGSPLLIGVKSEKKLKVDFVDVEFPEENAGQPEIPLKSNNKSFGLGPKKAREFEAGSQNANLLPIAANEFNLRHSQSRAFLSEDGSPTPVEFFVSSDAASVVKHTKKVLFLEDDDLAHIYDGELHIHRSRREVGASMTRSIQTLEMELAQIMKGPYDHFMQKEIYEQPESTFNTMRGRIDYENNKVILGGLKAWLPVVRRARRLIMIACGTSYHSCLATRAIFEELSDIPVSVELASDFLDRKCPVFRDDVCVFVSQSGETADTMLALNYCLERGALTVGIVNSVGSSISRVTHCGVHINAGPEIGVASTKAYTSQYIALVMFALSLSDDRVSKIDRRIEIIQGLKLIPGQIKQVLKLEPRIKKLCATELKDQKSLLLLGRGYQFAAALEGALKIKEISYMHSEGVLAGELKHGVLALVDENLPIIAFGTRDSLFPKVVSSIEQVTARKGHPIIICNENDEVWAQKSKSIDLQTLEVPQTVDCLQGLINIIPLQLMSYWLAVNKGIDVDFPRNLAKSVTVE

SEQ ID NO: 3 - protein, 717 aa, Saccharomyces cerevisiae YJM693
MCGIFGYCNYLVERSRGEIIDTLVDGLQRLEYRGYDSTGIAIDGDEADSTFIYKQIGKVSALKEEITKQNPNRDVTFVSHCGIAHTRWATHGRPEQVNCHPQRSDPEDQFVVVHNGIITNFRELKTLLINKGYKFESDTDTECIAKLYLHLYNTNLQNGHDLDFHELTKLVLLELEGSYGLLCKSCHYPNEVIATRKGSPLLIGVKSEKKLKVDFVDVEFPEENAGQPEIPLKSNNKSFGLGPKKAREFEAGSQNANLLPIAANEFNLRHSQSRAFLSEDGSPTPVEFFVSSDAASVVKHTKKVLFLEDDDLAHIYDGELHIHRSRREVGASMTRSIQTLEMELAQIMKGPYDHFMQKEIYEQPESTFNTMRGRIDYENNKVILGGLKAWLPVVRRARRLIMIACGTSYHSCLATRAIFEELSDIPVSVELASDFLDRKCPVFRDDVCVFVSQSGETADTMLALNYCLERGALTVGIVNSVGSSISRVTHCGVHINAGPEIGVASTKAYTSQYIALVMFALSLSDDRVSKIDRRIEIIQGLKLIPGQIKQVLKLEPRIKKLCATELKDQKSLLLLGRGYQFAAALEGALKIKEISYMHSEGVLAGELKHGVLALVDENLPIIAFGTRDSLFPKIVSSIEQVTARKGHPIIICNENDEVWAQKSKSIDLQTLEVPQTVDCLQGLINIIPLQLMSYWLAVNKGIDVDFPRNLAKSVTVE

SEQ ID NO: 4 - protein, 717 aa, Saccharomyces cerevisiae YJM1307
MCGIFGYCNYLVERSRGEIIDTLVDGLQRLEYRGYDSTGIAIDGDEADSTFIYKQIGKVSALKEEITKQNPNRDVTFVSHCGIAHTRWATHGRPEQVNCHPQRSDPEDQFVVVHNGIITNFRELKTLLINKGYKFESDTDTECIAKLYLHLYNTNLQNGHDLDFHELTKLVLLELEGSYGLLCKSCHYPNEVIATRKGSPLLIGVKSEKKLKVDFVDVEFPEENAGQPEIPLKSNNKSFGLGPKKAREFEAGSQNANLLPIAANEFNLRHSQSRAFLSEDGSPTPVEFFVSSDAASVAKHTKKVLFLEDDDLAHIYDGELHIHRSRREVGASMTRSIQTLEMELAQIMKGPYDHFMQKEIYEQPESTFNTMRGRIDYENNKVILGGLKAWLPVVRRARRLIMIACGTSYHSCLATRAIFEELSDIPVSVELASDFLDRKCPVFRDDVCVFVSQSGETADTMLALNYCLERGALTVGIVNSVGSSISRVTHCGVHINAGPEIGVASTKAYTSQYIALVMFALSLSDDRVSKIDRRIEIIQGLKLIPGQIKQVLKLEPRIKKLCATELKDQKSLLLLGRGYQFAAALEGALKIKEISYMHSEGVLAGELKHGVLALVDENLPIIAFGTRDSLFPKVVSSIEQVTARKGHPIIICNENDEVWAQKSKSIDLQTLEVPQTVDCLQGLINIIPLQLMSYWLAVNKGIDVDFPRNLAKSVTVE

SEQ ID NO: 5 - protein, 717 aa, Saccharomyces cerevisiae Pf-1
MCGIFGYCNYLVERSRGEIIDTLVDGLQRLEYRGYDSTGIAIDGDEADSTFIYKQIGKVSALKEEITKQNPNRDVTFVSHCGIAHTRWATHGRPEQVNCHPQRSDPEDQFVVVHNGIITNFRELKTLLINKGYKFESDTDTECIAKLYLHLYNTNLQNGHDLDFHELTKLVLLELEGSYGLLCKSCHYPNEVIATRKGSPLLIGVKSEKKLKVDFVDVEFPEENAGQPEIPLKSNNKSFGLGPKKAREFEAGSQNANLLPIAANEFNLRHSQSRAFLSEDGSPTPVEFFVSSDAASVVKHTKKVLFLEDDDLAHIYDGELHIHRSRREVGASMTRSIQTLEMELAQIMMGPYDHFMQKEIYEQPESTFNTMRGRIDYENNKVILGGLKAWLPVVRRARRLIMIACGTSYHSCLATRAIFEELSDIPVSVELASDFLDRKCPVFRDDVCVFVSQSGETADTMLALNYCLERGALTVGIVNSVGSSISRVTHCGVHINAGPEIGVASTKAYTSQYIALVMFALSLSDDRVSKIDRRIEIIQGLKLIPGQIKQVLKLEPRIKKLCATELKDQKSLLLLGRGYQFAAALEGALKIKEISYMHSEGVLAGELKHGVLALVDENLPIIAFGTRDSLFPKVVSSIEQVTARKGHPIIICNENDEVWAQKSKSIDLQTLEVPQTVDCLQGLINIIPLQLMSYWLAVNKGIDVDFPRNLAKSVTVE

SEQ ID NO: 6 - protein, 717 aa, Saccharomyces cerevisiae YJM456
MCGIFGYCNYLVERSRGEIIDTLVDGLQRLEYRGYDSTGIAIDGDEADSTFIYKQIGKVSALKEEITKQNPNRDVTFVSHCGIAHTRWATHGRPEQVNCHPQRSDPEDQFVVVHNGIITNFRELKTLLINKGYKFESDTDTECIAKLYLHLYNTNLQNGHDLDFHELTKLVLLELEGSYGLLCKSCHYPNEVIATRKGSPLLIGVKSEKKLKVDFVDVEFPEENAGQPEIPLKSNNKSFGLGPKKAREFEAGSQNANLLPIAANEFNLRHSQSRAFLSEDGSPTPVEFFVSSDAASVVKHTKKVLFLEDDDLAHIYDGELHIHRSRREVGASMTRSIQTLEMELAQIMKGPYDHFMQKEIYEQPESTFNTMRGRIDYENNNVILGGLKAWLPVVRRARRLIMIACGTSYHSCLATRAIFEELSDIPVSVELASDFLDRKCPVFRDDVCVFVSQSGETADTMLALNYCLERGALTVGIVNSVGSSISRVTHCGVHINAGPEIGVASTKAYTSQYIALVMFALSLSDDRVSKIDRRIEIIQGLKLIPGQIKQVLKLEPRIKKLCATELKDQKSLLLLGRGYQFAAALEGALKIKEISYMHSEGVLAGELKHGVLALVDENLPIIAFGTRDSLFPKVVSSIEQVTARKGHPIIICNENDEVWAQKSKSIDLQTLEVPQTVDCLQGLINIIPLQLMSYWLAVNKGIDVDFPRNLAKSVTVE

SEQ ID NO: 7 - protein, 717 aa, Saccharomyces cerevisiae NYR20
MCGIFGYCNYLVERSRREIIDTLVDGLQRLEYRGYDSTGIAIDGDEADSTFIYKQIGKVSALKEEITKQNPNRDVTFVSHCGIAHTRWATHGRPEQVNCHPQRSDPEDQFVVVHNGIITNFRELKTLLINKGYKFESDTDTECIAKLYLHLYNTNLQNGHDLDFHELTKLVLLELEGSYGLLCKSCHYPNEVIATRKGSPLLIGVKSEKKLKVDFVDVEFPEENAGQPEIPLKSNNKSFGLGPKKAREFEAGSQNANLLPIAANEFNLRHSQSRAFLSEDGSPTPVEFFVSSDAASVVKHTKKVLFLEDDDLAHIYDGELHIHRSRREVGASMTRSIQTLEMELAQIMKGPYDHFMQKEIYEQPESTFNTMRGRIDYENNKVILGGLKAWLPVVRRARRLIMIACGTSYHSCLATRAIFEELSDIPVSVELASDFLDRKCPVFRDDVCVFVSQSGETADTMLALNYCLERGALTVGIVNSVGSSISRVTHCGVHINAGPEIGVASTKAYTSQYIALVMFALSLSDDRVSKIDRRIEIIQGLKLIPGQIKQVLKLEPRIKKLCATELKDQKSLLLLGRGYQFAAALEGALKIKEISYMHSEGVLAGELKHGVLALVDENLPIIAFGTRDSLFPKVVSSIEQVTARKGHPIIICNENDEVWAQKSKSIDLQTLEVPQTVDCLQGLINIIPLQLMSYWLAVNKGIDVDFPRNLAKSVTVE

SEQ ID NO: 8 - protein, 717 aa, Saccharomyces paradoxus CBS432
MCGIFGYCNYLVERSRGEIIDTLVDGLQRLEYRGYDSTGIAIDGDEADSTFIYKQIGKVSALKEEITKQNPNRDVTFVSHCGIAHTRWATHGQPEQVNCHPQRSDPEDQFVVVHNGIITNFRELKTLLINKGYKFESDTDTECIAKLYLHLYNTNLQNGHDLDFHELTKLVLLELEGSYGLLCKSCHYPNEVIATRKGSPLLIGVKSEKKLKVDFVDVEFPEENAGQPEIPLKSNNKSFGLGPKKAREFEAGSQNANLLPIAANEFNLRHSQSRAFLSEDGSPTPVEFFVSSDAASVVKHTKKVLFLEDDDLAHIYDGELHIHRSRREVGASMTRSIQTLEMELAQIMKGPYDHFMQKEIYEQPESTFNTMRGRIDYENNKVILGGLKAWLPVIRRARRLIMIACGTSYHSCLATRAIFEELSDIPVSVELASDFLDRKCPVFRDDVCVFVSQSGETADTMLALNYCLERGALTVGIVNSVGSSISRVTHCGVHINAGPEIGVASTKAYTSQYIALVMFALSLSDDRVSKIDRRIEIIQGLKLIPGQIKQVLKLEPRIKKLCATELKDQKSLLLLGRGYQFAAALEGALKIKEISYMHSEGVLAGELKHGVLALVDENLPIIAFGTRDSLFPKVVSSIEQVTARKGHPIIICNENDEVWAQKSKSIDLQTLEVPQTVDCLQGLINIIPLQLMSYWLAVNKGIDVDFPRNLAKSVTVE

SEQ ID NO: 9 - protein, 717 aa, Saccharomyces cerevisiae YJM1311
MCGIFGYCNYLVERSRREIIDTLVDGLQRLEYRGYDSTGIAIDGDEADSTFIYKQIGKVSALKEEITKQNPNRDVTFVSHCGIAHTRWATHGRPEQVNCHPQRSDPEDQFVIVHNGIITNFRELKTLLINKGYKFESDTDTECIAKLYLHLYNTNLQNGHDLDFHELTKLVLLELEGSYGLLCKSCHYPNEVIATRKGSPLLIGVKSEKKLKVDFVDVEFPEENAGQPEIPLKSNNKSFGLGPKKAREFEAGSQNANLLPIAANEFNLRHSQSRAFLSEDGSPTPVEFFVSSDAASVVKHTKKVLFLEDDDLAHIYDGELHIHRSRREVGASMTRSIQTLEMELAQIMKGPYDHFMQKEIYEQPESTFNTMRGRIDYENNKVILGGLKAWLPVVRRARRLIMIACGTSYHSCLATRAIFEELSDIPVSVELASDFLDRKCPVFRDDVCVFVSQSGETADTMLALNYCLERGALTVGIVNSVGSSISRVTHCGVHINAGPEIGVASTKAYTSQYIALVMFALSLSDDRVSKIDRRIEIIQGLKLIPGQIKQVLKLEPRIKKLCATELKDQKSLLLLGRGYQFAAALEGALKIKEISYMHSEGVLAGELKHGVLALVDENLPIIAFGTRDSLFPKVVSSIEQVTARKGHPIIICNENDEVWAQKSKSIDLQTLEVPQTVDCLQGLINIIPLQLMSYWLAVNKGIDVDFPRNLAKSVTVE

SEQ ID NO: 10 - protein, 717 aa, Saccharomyces cerevisiae P-684
MCGIFGYCNYLVERSRREIIDTLVDGLQRLEYRGYDSTGIAIDGDEADSTFIYKQIGKVSALKEEITKQNPNRDVTFVSHCGIAHTRWATHGRPEQVNCHPQRSDPEDQFVVVHNGIITNFRELKTLLINKGYKFESDTDTECIAKLYLHLYNTNLQNGHDLDFHELTKLVLLELEGSYGLLCKSCHYPNEVIATRKGSPLLIGVKSEKKLKVDFVDVEFPEENAGQPEIPLKSNNKSFGLGPKKAREFEAGSQNANLLPIAANEFNLRHSQSRAFLSEDGSPTPVEFFVSSDAASVAKHTKKVLFLEDDDLAHIYDGELHIHRSRREVGASMTRSIQTLEMELAQIMKGPYDHFMQKEIYEQPESTFNTMRGRIDYENNKVILGGLKAWLPVVRRARRLIMIACGTSYHSCLATRAIFEELSDIPVSVELASDFLDRKCPVFRDDVCVFVSQSGETADTMLALNYCLERGALTVGIVNSVGSSISRVTHCGVHINAGPEIGVASTKAYTSQYIALVMFALSLSDDRVSKIDRRIEIIQGLKLIPGQIKQVLKLEPRIKKLCATELKDQKSLLLLGRGYQFAAALEGALKIKEISYMHSEGVLAGELKHGVLALVDENLPIIAFGTRDSLFPKVVSSIEQVTARKGHPIIICNENDEVWAQKSKSIDLQTLEVPQTVDCLQGLINIIPLQLMSYWLAVNKGIDVDFPRNLAKSVTVE

SEQ ID NO: 11 - protein, 716 aa, S.cerevisiae strain S288C
MCGIFGYCNYLVERSRGEIIDTLVDGLQRLEYRGYDSTGIAIDGDEADSTFIYKQIGKVSALKEEITKQNPNRDVTFVSHCGIAHTRWATHGRPEQVNCHPQRSDPEDQFVVVHNGIITNFRELKTLLINKGYKFESDTDTECIAKLYLHLYNTNLQNGHDLDFHELTKLVLLELEGSYGLLCKSCHYPNEVIATRKGSPLLIGVKSEKKLKVDFVDVEFPEENAGQPEIPLKSNNKSFGLGPKKAREFEAGSQNANLLPIAANEFNLRHSQSRAFLSEDGSPTPVEFFVSSDAASVVKHTKKVLFLEDDDLAHIYDGELHIHRSRREVGASMTRSIQTLEMELAQIMKGPYDHFMQKEIYEQPESTFNTMRGRIDYENNKVILGGLKAWLPVVRRARRLIMIACGTSYHSCLATRAIFEELSDIPVSVELASDFLDRKCPVFRDDVCVFVSHCGETADTMLALNYCLERGALTVGIVNSVGSSISRVTHCGVHINAGPEIGVASTKAYTSQYIALVMFALSLSDDRVSKIDRIEIIQGLKLIPGQIKQVLKLEPRIKKLCATELKDQKSLLLLGRGYQFAAALEGALKIKEISYMHSEGVLAGELKHGVLALVDENLPIIAFGTRDSLFPKVVSSIEQVTARKGHPIIICNENDEVWAQKSKSIDLQTLEVPQTVDCLQGLINIIPLQLMSYWLAVNKGIDVDFPRNLAKSVTVE

SEQ ID NO: 12 - protein, 717 aa, Saccharomyces kudriavzevii IFO 1802
MCGIFGYCNYLVERSRGDIIDTLVDGLQRLEYRGYDSTGIAIDGDEADSTFIYKQIGKVSALKEEIAKQKPNRDVTFVSHCGIAHTRWATHGRPEKRNCHPQRSDPEGQFVVVHNGIITNFRELKTLLANKGYKFESDTDTECIAKLYLHLYNTNLQNGHDLDFHELTKLVLLELEGSYGLLCKSCHYPNEVIATRKGSPLLIGVKSEKKLKVDFVDVEFPEDNAGQPEIPLKSNNKSFGLGPRKAREFEAGSQNANLLPIAPNEFNLRHSQSRAFLSEDGSPTPVEFFVSSDAASVVKHTKKVLFLEDDDLAHIYDGELHIHRSRREVGASMTRSIQTLEMELAQIMKGPYDHFMQKEIYEQPESTFNTMRGRIDYDNNKVILGGLKAWLPVVRRARRLIMIACGTSYHSCLATRAIFEELSDIPVSVELASDFLDRKCPVFRDDVCVFVSQSGETADTILALNYCLERGALTVGIVNSVGSSISRVTHCGVHINAGPEIGVASTKAYTSQYIALVMFALSLSDDRVSKIDRRIEIIQGLKLIPGQIKQVLKLEPRIKKICSTELKDQKSLLLLGRGYQFAAALEGALKIKEISYMHSEGVLAGELKHGVLALVDENLPIIAFGTRDSLFPKVVSSIEQVTARKGHPIIICNENDEVWAEKSKSIDLQTLEVPQTVDCLQGLINIVPLQLMSYWLAVEKGIDVDFPRNLAKSVTVE

SEQ ID NO: 13 - protein, 717 aa, Saccharomyces cerevisiae x Saccharomyces kudriavzevii VIN7
MCGIFGYCNYLVERSRGDIIDTLVDGLQRLEYRGYDSTGIAIDGDEADSTFIYKQIGKVSALKEEIAKQKPNRDVTFVSHCGIAHTRWATHGRPEKRNCHPQRSDPEGQFVVVHNGIITNFRELKTLLANKGYKFESDTDTECIAKLYLHLYNTNLQNGHDLDFHELTKLVLLELEGSYGLLCKSCHYPNEVIATRKGSPLLIGVKSEKKLKVDFVDVEFPEDNAGQPETPLKSNNKSFGLGPRKAREFEAGSQNANLLPIAPNEFNLRHSQSRAFLSEDGSPTPVEFFVSSDAASVVKHTKKVLFLEDDDLAHIYDGELHIHRSRREVGASMTRSIQTLEMELAQIMKGPYDHFMQKEIYEQPESTFNTMRGRIDYDNNKVILGGLKAWLPVVRRARRLIMIACGTSYHSCLATRAIFEELSDIPVSVELASDFLDRKCPVFRDDVCVFVSQSGETADTILALNYCLERGALTVGIVNSVGSSISRVTHCGVHINAGPEIGVASTKAYTSQYIALVMFALSLSDDRVSKIGRRIEIIQGLKLIPGQIKQVLKLEPRIKKICSTELKDQKSLLLLGRGYQFAAALEGALKIKEISYMHSEGVLAGELKHGVLALVDENLPIIAFGTRDSLFPKVVSSIEQVTARKGHPIIICNENDEVWAEKSKSIDLQTLEVPQTVDCLQGLINIVPLQLMSYWLAVEKGIDVDFPRNLAKSVTVE

SEQ ID NO: 14 - protein, 717 aa, Saccharomyces pastorianus strain CBS 1483
MCGIFGYCNYLVERSRGEIIDTLVDGLQRLEYRGYDSTGIAIDGDEVDSTLIYKQIGKVSALKEEIAREKPNRDVTFVSHCGIAHTRWATHGQPEKINCHPQRSDPESQFVIVHNGIITNFRELKTLLLNKGYKFESDTDTECIAKLYLHLYNTNLQNGHDLDFHELTKLVLLELEGSYGLLCKSCHYPNEVIATRKGSPLLIGVKSDKKLKVDFVDVEFPEDNAGQPETPLRSNNRPFGLGPKKAREFEAGSQNGNLLPIAANEFNLRHSQSRAFLSEDGSPTPVEFFVSSDAASVVKHTKKVLFLEDDDLAHIYDGELHIHRSRREVGASMTRSIQTLEMELAQIMKGPYDHFMQKEIYEQPESTFNTMRGRIDFDNNKVILGGLKAWLPVVRRARRLIMIACGTSYHSCLATRAIFEELSDIPVSVELASDFLDRKCPVFRDDVGVFVSQSGETADTMLALNYCLERGALTVGIVNSVGSSISRVTHCGVHINAGPEIGVASTKAYTSQYIALVMFALSLSDDRVSKVDRRIEIIQGLKMIPGQIKQVLNLEPRIKELCATELKDQKSLLLLGRGYQFAAALEGALKIKEISYMHSEGVLAGELKHGVLALVDENLPIIAFGTRDSLFPKVVSSIEQVTARKGHPIIICNENDEVWAEKSKTIKLQTLEVPQTVDCLQGLVNIIPLQLMSYWLAVNKGIDVDFPRNLAKSVTVE

SEQ ID NO: 15 - protein, 724 aa, Naumovozyma castellii CBS 4309
MCGIFGYCNFLVERSRGEIIDTLVEGLERLEYRGYDSTGIAIDGDELDSTMIFRQIGKVAALKEEIKKQNPNRDVTFVSHCGIAHTRWATHGEPKQVNCHPQRSDPLNSFVIVHNGIITNFRELRTLLVNKGFKFESDTDTECIAKLFKHLYDTNLQNGNELDFHELTKLVLLELEGSYGLLCKSIHYPNEVIATRKGSPLLIGVKSEKKLKVDFVDVEFPDDNNLGKPETPLGSATDSTVDGITVNGKKHPRSYGVDEAQSSNLLPIAANEFNLRHSQSRAFLSEDGTPNPVEFFLSSDAASVIKHTKKVLFLEDDDIAHIYDGELHIHRSRREVGASMTRSIQTLEMELAQIMKGPYKHFMQKEIYEQPESTLNTMRGRIDFENNTLMLGGLKAWLPAIRRARRLIMIACGTSYHSCLATRAIFEELSEIPVSVELASDFLDRKSPVFRDDICVFVSQSGETADTMLALNYCLERGALTVGIVNSVGSSLSRATHCGVHINAGPEIGVASTKAYTSQYIALVMFALSLSDDRLSKLERRKDIIQGLKLIPGQIKQVLQLEKKIKKLCENELKDQKSLLLLGRGYQFASALEGALKIKEISYMHSEGVLAGELKHGVLALVDENLPIIAFGTRDSLFPKVVSSIEQVTARKGHPIVICNENDEVWAKKAESIHLVTLEVPQTVDCLQGLLNIIPLQLMSYWLAVNKGIDVDFPRNLAKSVTVE

SEQ ID NO: 16 - protein, 723 aa, Candida glabrata strain DSY562
MCGIFGYCNYLVEKSRGEIIDTLVDGLKRLEYRGYDSTGIAIDGDELDSTFIFRQIGKVSSLEKEIALQKPNRDVTFVSHSGIAHTRWATHGEPKQINCHPQRSDPNNEFVIVHNGIITNFRELKTLLLNKGYQFESDTDTECIAKLFKHLYDTNLKNGHDLDFHELTKLVLLELEGSYGLLCKSNHYPNEIIATRKGSPLLVGVKSEKKLKVDFVDVEFPDESMGKPDTPLVSASGEAINNNNVTVSGRNARHVEANAHNNNLMPIAANDFNLRHSQSRAFLSEDGSPTPVEFFVSSDAASVIKHTKKVLFLEDDDIAHIYDGELHIHRSRKEVGASMTRSIQTLEMELAQIMKGPYKHFMQKEIYEQPESSFNTMRGRIDFENKNVMLGGLKAWLPVIRRARRLIMIACGTSYHSCLATRAIFEELSEIPVSVELASDFLDRRSPVFREDVCIFVSQSGETADTMFALNYCLERGALTIGIVNTVGSSISRVTHCGVHINAGPEIGVASTKAYTSQYVALVMVALSLSDDRVSKIERRKEIIDGLKLIPGQIKQVLNLEDKIKKLCVTELKDQKSLLLLGRGYQFASALEGALKIKEISYMHSEGVLAGELKHGVLALVDENLPIIAFGTRDSLFPKVVSSIEQVTARKGKPIIICNENDDVWAKKAEQINLVTLEVPQTVDCLQGLLNIIPLQLMSYWLAVNKGIDVDFPRNLAKSVTVE

SEQ ID NO: 17 - protein, 723 aa, Candida glabrata strain BG2
MCGIFGYCNYLVEKSRGEIIDTLVDGLKRLEYRGYDSTGIAIDGDELDSTFIFRQIGKVSSLEKEIALQKPNRDVTFVSHSGIAHTRWATHGEPKQINCHPQRSDPNNEFVIVHNGIITNFRELKTLLLNKGYQFESDTDTECIAKLFKHLYDTNLKNGHDLDFHELTKLVLLELEGSYGLLCKSSHYPNEIIATRKGSPLLVGVKSEKKLKVDFVDVEFPDESMGKPDTPLVSASGEAINNNNVTVSGRNARHVEANAHNNNLMPIAANDFNLRHSQSRAFLSEDGSPTPVEFFVSSDAASVIKHTKKVLFLEDDDIAHIYDGELHIHRSRKEVGASMTRSIQTLEMELAQIMKGPYKHFMQKEIYEQPESSFNTMRGRIDFENKNVMLGGLKAWLPVIRRARRLIMIACGTSYHSCLATRAIFEELSEIPVSVELASDFLDRRSPVFREDVCIFVSQSGETADTMFALNYCLERGALTIGIVNTVGSSISRVTHCGVHINAGPEIGVASTKAYTSQYVALVMVALSLSDDRVSKIERRKEIIDGLKLIPGQIKQVLNLEDKIKKLCVTELKDQKSLLLLGRGYQFASALEGALKIKEISYMHSEGVLAGELKHGVLALVDENLPIIAFGTRDSLFPKVVSSIEQVTARKGKPIIICNENDDVWAKKAEQINLVTLEVPQTVDCLQGLLNIIPLQLMSYWLAVNKGIDVDFPRNLAKSVTVE

SEQ ID NO: 18 - protein, 727 aa, Naumovozyma dairenensis CBS 421
MCGIFGYCNYLVERSRGEIIDTLVDGLERLEYRGYDSTGLAIDGDEPDSTMIFKQIGKVSALKKEIKRQNPNRDVTFVSHCGIAHTRWATHGEPKQVNCHPQRSDPNNEFVVVHNGIITNFRELKTLLTNKGFKFESDTDTECIAKLFKHIYDTNVQNGNDLDFHELTKLVLLELEGSYGLLCRSSHYPNEVIATRKGSPLLIGVKSEKKLKVDFVDVEFPDDNRPETPIHNGTSNHINGSRIPNGNGPTTTNGNSNHINGLKKAHNLLPIATNEFNLRHSQSRAFLSEDGTPNPVEFFLSSDAASVIKHTKKVLFLEDDDLAHIYDGELHIHRSRKEVGASMTRSIQTLEMELAQIMKGPYKHFMQKEIYEQPESTLNTMRGRIDFDNNSIMLGGLKAWLPAIRRARRLIMVACGTSYHSCLATRAIFEELSEIPVSVELASDFLDRKSPVFRDDICVFVSQSGETADTMLALNYCLERGALTVGIVNSVGSSLSRATHCGVHINAGPEIGVASTKAYTSQYIALVMVALSLSDDRVSKLERRKDIIQGLKLIPGQIKQVLNLEDRIKKLCENELKDQKSLLLLGRGYQFASALEGALKIKEISYMHSEGVLAGELKHGVLALVDENLPIIAFGTRDSLFPKVVSSIEQVTARKGHPIVICNENDDVWAKKAESVHLETLEVPQTVDCLQGLLNIIPLQLMSYWLAVNKGIDVDFPRNLAKSVTVE

SEQ ID NO: 19 - protein, 725 aa, Torulaspora delbrueckii CBS 1146
MCGIFGYCNYLVEKSRGEIIDTLVDGLQRLEYRGYDSTGIAIDGDEPDSTLVYKQIGKVSALKEEIAEHHPKRDVTFVSHCGIAHTRWATHGEPKQVNCHPQKSDPTGEFVIVHNGIITNFRELKTLLSNKGYSFESDTDTECIAKLFKHLYDTNIQNGNELDFHELTKLVLLELEGSYGLLCRSSHYPDEVMATRKGSPLLIGVKSEKKLKVDFVDVEFPDDNAQPETPLVSERSNGLASSRNLTVNGGRKVRLFDHEDRGNSNLLPIAANEFNLRHSQSRAFLSEDGTPNPVEFFLSSDASSVIKHTKKVLFLEDNDIAHIYDGEIHIHRSRREVGASMTRSIQTLEMELAQIMKGPYKHFMQKEIFEQPESTLNTMRGRIDFENNSVMLGGLQAWLPAIRRARRLIMIACGTSYHSCLATRAIFEELSEIPVSVELASDFLDRKSPVFRDDICVFVSQSGETADTMLALNYCLERGALTVGIVNSVGSSISRITHCGVHINAGPEIGVASTKAYTSQYIALVMVALSLSDDRVSKIERRGDIMQGLKKIPQQIKEVLQLEGKIKQLCEKDLKDQKSLLLLGRGYQFASALEGALKIKEISYMHSEGVLAGELKHGVLALVDEDLPIIAFGTRDSLFPKVVSSIEQVTARKGHPIVICNKNDEVWEKKAATTKMVTLEVPQTVDCLQGLLNIIPLQLISYWLAVNKGIDVDFPRNLAKSVTVE

SEQ ID NO: 20 - protein, 727 aa, Torulaspora globosa strain CBS764
MCGIFGYCNYLVEKSRGDIIDTLVEGLQRLEYRGYDSTGIAIDGDELDSTLIYKQIGKVSALREEIAEHKPKRDVTFLSHCGIAHTRWATHGEPKQINCHPQKSDPNCEFVIVHNGIITNYRELKTLLLNKGYTFESDTDTECIAKLFKHLYDTNLENGHELDFHELTKLVLLELEGSYGLLCRSAHYPNEVIATRKGSPLLIGVKSEKKLKVDFVDVEFPDDNAQPETPLVSERANGATANGNSVRTLNGSGKKRLYDHEDRGNSNLLPIAANEFNLRHSQSRAFLSEDGTPNPVEFFLSSDASSVIKHTKKVLFLEDNDIAHIYDGEIHIHRSRREVGASMTRSIQTLEMELAQIMKGEYNHFMQKEIFEQPESAFNTMRGRIDFEKGTVMLGGLKAWLPAIRRARRLVMIACGTSYHSCLATRAIFEELSEIPVSVELASDFLDRKSPIFRDDICVFVSQSGETADTMLALNYSLERGALTVGIVNSVGSSISRITHCGVHINAGPEIGVASTKAYTSQYIALVMFALSLSDDRVSKLERRQDIIRGLKMIPEQIKEVLSLEPQIKALCEKELKDQKSLLLLGRGYQFASALEGALKIKEISYMHSEGVLAGELKHGVLALVDEDLPIIAFGTRDSLFPKVVSSIEQVTARKGQPIIICNKNDEAWEKKSATTKMVTLEVPLTVDCLQGLLNIIPLQLISYWLAVNKGIDVDFPRNLAKSVTVE

SEQ ID NO: 21 - protein, 721 aa, Zygosaccharomyces rouxii BRC 110957
MCGIFGYCNYLVERSRGEIIDTLVEGLQRLEYRGYDSTGIAIDGDTPDSTLLYKQIGKVDALKQEIAQDAPERDITFASHCGIAHTRWATHGEPRQINCHPQRSDPQNEFTVVHNGIITNFRELRTLLANRGYTFESDTDTECIAILFKHLYDENLSNGHELDFHELTKLVLTELEGSYGLLCKSLHYPEEVIATRKGSPLLIGVKSDKKLKVDFVDIEFPDDSAGKPDTPLGSRSGTPNASSRNLLNGNRRTRPYDGHNLLPLAANEFNLRHSQSRAFLSEDGTPNPVEFFVSSDAASVIKHTKKVLFLEDDDIAHIYDGELHIHRSRRPEVGASMTRSIQTLEMELAQIMKGPYKHFMQKEIFEQPDSALNTMRGRIDFENGTIMLGGLKAWLPAVRRARRLIMIACGTSYHSCLATRAMFEELSEIPVSVELASDFLDRKCPVFRDDVCCFVSQSGETADTMLALNYCLERGALTLGIVNSVGSSISRATHCGVHINAGPEIGVASTKAYTSQYIALVMFALSLSDDRVSKIDRRKEIIKGLQKIPDQIREVLKLETRIKELCQNDLKDHKSLLLLGRGYQFASALEGALKIKEISYMHSEGVLAGELKHGVLALVDEQLPIIAFGTRDSLFPKVVSSIEQVTARKGRPIIICNTNDEVWAKKAETNKLLTLEVPQTVDCLQGLLNIIPLQLISYWLAVNRGIDVDFPRNLAKSVTVE

SEQ ID NO: 22 - protein, 706 aa, Ashbya gossypii FDAG1
MCGIFGYCNYLVEKSRGEIIDTLVEGLQRLEYRGYDSTGIAIDGDGADETFIFKQIGKVSALEREIAERAPARDVRFVSHCGIAHTRWATHGQPRQVNCHPHRSDARNEFVVVHNGIITNFRELKTLLRNKGYEFESDTDTEVIAKLFKHLYDTNLENGHELDFHELTKQVLLELEGSYGLLCRSSRYPCEVIATRKGSPLLVGVKSERKLKVDFVDVEFPDNEKQPDVPIPAVLNGHKAGADCEKLLPIAASETGLRHSQSRAFLSEDGVPNPVEFFLSSDAASVIKHTKKVLFLEDDDIAHIYDGELHIHRSRREVGASATRSIQTLEMELAQIMKGPYDHFMQKEIFEQPESTLNTMRGRIDFENNTVMLGGLKSWLPAIRSARRLTMIACGTSYHSCLATRAIFEELSEIPVSIELASDFLDRRCPVFRDDVCIFVSQSGETADTMLALNYCIERGALTVGIVNSVGSSISRVTHCGVHINAGPEIGVASTKAYTSQYIALVMLALSLSDDRVSRIERRKEIIQGLKEIPRQIKQVLQLEKQIKHLCETELCNQKSLLLLGRGYQFASALEGALKIKEISYMHSEGVLAGELKHGVLALVDENLPIIAFGTRDSLFPKVVSSIEQVTARKGRPIIICNENDEVWTRKAQNSNLLTLEVPLTVDCLQGLLNIIPLQLTAYWLAVNNGIDVDFPRNLAKSVTVE

SEQ ID NO: 23 - protein, 701 aa, Lachancea quebecensis LAQU0
MCGIFGYCNYLVEKSRGDVIDTLVEGLQRLEYRGYDSTGIAIDGDEPDSTLIYKQIGKVAALEKEIEVEKPNRDVTFVSHCGIAHTRWATHGEPKWANCHPQRSNMASDFVIVHNGIITNFRELKQLLINKGYKFESDTDTECIAKLFKHLYDTNLKHGNEPDFHELTMQVLLELDGSYGLLCRSVHYPNEVIATRKGSPLLIGVKSERKLKVDFVDVEFPDTDEAQPDIPLNSSRGEARNFLPIASNESALRRSQSRAFLSEDGAPNPVEFFVSSDAASVVKHTKKVLFLEDDDIAHIYDGELHIHRSRREIGASATRSIQTLEMELAQIMKGSYKHFMQKEIFEQPDSTFNTMRGRIDFESNSVMLGGLNSWLPVIRRARRLIMIACGTSYHSCLATRAIFEELSEIPVSVELASDFLDRKTPVFRDDIAVFVSQSGETADTMLALKYCLERGALTVGIVNNVGSSISRVTHCGVHINAGPEIGVASTKAYTSQYIALVMFALSLSDDRVSKVERRREIIQGLKKIPEQIRKVLLMEPRIKELCEKELKDQKSLLLLGRGYQFASALEGALKIKEISYMHSEGVLAGELKHGVLALVDENLPIIAFGTRDSLFPKVVSSIEQVTARKGRPIIICNENDEVWSAKAANSNLATLEVPLTVDCLQGLLNIIPLQLTSYWLAVNKNIDVDFPRNLAKSVTVE

SEQ ID NO: 24 - protein, 726 aa, Kazachstania naganishii CBS 8797
MCGIFGYCNFLVEKSRGEILDTLVEGLQRLEYRGYDSTGVAIDGDELDSTTIIKQIGKVDALKEEIAKVNPNRDVTFASHVGIAHTRWATHGQPRQVNCHPQRSDANNEFVIVHNGIITNFRELKTLLTNKGYKFESETDTECIAKLFKHIYDTNTQNGNELDFHELTKLVLLELEGSYGLLCRSSHYPDEVIATRKGSPLLIGVKSEKKLKMDFVDVEFPDDTENRPETPLRGNTPTGRNGAISVTSNKKNPNAFISNSHSDNDNLLPIAANHFNLRHSQSRAFLSEDGSPAPMEFFLSSDASSVIKHTKKVLFLEDDDIAHIYDGELHIHRSRREVGASMTRSIQTLEMELAQIMKGRYKHFMQKEIYEQPESTFNTMRGRIDFENNRITLGGLKAWLPAIRRARRLIMIACGTSYHSCLATRAIFEELSDIPVSVELASDFLDRKSPVFRDDICIFVSQSGETADTMLALNYCLERGSLTVGIVNSVGSSLSRATHCGVHINAGPEIGVASTKAYTSQYIALVMVALSLSDDRVSKLQRRNDIIQGLKQIPQQIKKVLDLEPKIKKLCQNKLKNQKSLLILGRGYQFASALEGALKIKEISYMHSEGVLAGELKHGVLALVDENLPIIAFGTRDSLFPKVVSSIEQITARKGHPIIICNDNDEVWSKKAEGTKLETLNVPQTVDCLQGLLNIVPLQLISYRLAVDKGIDVDFPRNLAKSVTVE

SEQ ID NO: 25 - protein, 707 aa, Saccharomycetaceae sp. ′Ashbya aceri′
MCGIFGYCNYLVEKSRGEIIDTLVEGLQRLEYRGYDSTGIAIDGDAADETFIFKQIGKVSALEREIAERAPARDARFVSHCGIAHTRWATHGQPRQVNCHPHRSDAGNEFVVVHNGIITNFRELKTLLRNKGYQFESDTDTEVIAKLFKHLYDTNLENGHELDFHELTKQVLLELEGSYGLLCRSSRYPAEVIATRKGSPLLVGVKSERKLKVDFVDVEFPDNDKQPDVPIPAVSNGHKAGADGRDKLLPIAASETGLRHSQSRAFLSEDGVPNPVEFFLSSDAASVIKHTKKVLFLEDDDIAHIYDGELHIHRSRREVGASATRSIQTLEMELAQIMKGPYDHFMQKEIFEQPESTLNTMRGRIDFENNTVMLGGLKSWLPAIRSARRLTMIACGTSYHSCLATRAIFEELSEIPVSIELASDFLDRRCPVFRDDVCIFVSQSGETADTMLALNYCIERGALTVGIVNSVGSSISRVTHCGVHINAGPEIGVASTKAYTSQYIALVMLALSLSDDRVSRIERRKEIIQGLKEIPSQIKQVLQLEKQIKHLCETELCNQKSLLLLGRGYQFASALEGALKIKEISYMHSEGVLAGELKHGVLALVDENLPIIAFGTRDSLFPKVVSSIEQVTARKGRPIIICNQNDEVWTKKAQNSNLLTLEVPLTVDCLQGLLNIIPLQLTAYWLAVNNGIDVDFPRNLAKSVTVE

SEQ ID NO: 26 - protein, 701 aa, Lachancea fermentati
MCGIFGYCNYLVEKSRGDVIDTLVEGLQRLEYRGYDSTGIAIDGDEPDSTLIYKQIGKVKALEKEIEEHHPNREVTFVTHCGIAHTRWATHGEPVQINCHPQRSDRHNEFVIVHNGIITNFRELKQLLLNKGYTFESETDTECIAKLFKHMYDTNLKNGVELDFHDLTKQVLLELEGSYGLLCRSAHYPNEVIATRKGSPLLIGVKSERKLKVDFVDVEFPDTDDVQPDIPLNSARTEDRNLLPIAANESALRRSQSRAFLSEDGAPNPVEFFVSSDAASVIKHTKKVLFLEDDDIAHIYDGELHIHRSRREMGASAIRSIQTLEMELAQIMKGPYKHFMQKEIFEQPESTLNTMRGRIDFENNTVMLGGLKSWLPIIRRTRRLIMIACGTSYHSCLATRAIFEELSEIPVSVELASDFLDRKTPVFRDDVCVFVSQSGETADTMLALKYCLERGALTVGIVNNVGSSISRVTHCGVHINAGPEIGVASTKAYTSQYIALVMLALSLSDDKVSRVERRKEIIQGLKEIPEQIKQVLKMEPRIKELCETELRDKRSLLLLGRGYQFASALEGALKIKEISYMHSEGVLAGELKHGVLALVDENLPIIAFGTRDSLFPKVVSSIEQVTARKGRPIIICNENDDLWSKRASQSNLVTLEVPLTVDCLQGLLNIIPLQLTSYWLAVNKGIDVDFPRNLAKSVTVE

SEQ ID NO: 27 - protein, 718 aa, Tetrapisispora blattae CBS 6284
MCGIFGYCNYLVEKTRGEIIDTLVEGLERLEYRGYDSTGIAIDGDEPDSIDIFREIGKVSSLKEQIKKANPNRDVTYLTHSGIAHTRWATHGQPKQVNCHPQRSDAENQFVIIHNGIITNFRELKTLLMNKGYTFESDTDTECIAKLFLHLYQTNLKSGHELDFHELSKLVLMELEGSYGLLCKSIHYPGEVIATRKGSPLLIGVKSEKKLKVDFVDVEFPDENAAQPETPMTINEPKVIKSSSLKSNKSDFHSKDGLMPIAANEFNLRHSQSRAFLSEDGAPNPIEFFISSDAASVIKYTKKVLFLEDDDIAHIYDGELHIHRSRKDVGGSMTRSIQTLELELAQIMKGPYKHFMQKEIYEQPESTYNTMRGRLDFDNNLVNLGGLQQFIPTIRRARRLIMIACGTSYHSCLATRAIFEEFSDLPVSIELASDFLDRKCPVFRDDVCVFVSQSGETADTMFALNYVMERGALTVGIVNTVGSSISRATNCGVHINAGPEIGVASTKAYTSQYIALVMFAVLLGEDRISKTERRKEIIQGLKLIPEQIKQVLKLEPRIKQLCENELSDQKSLLLLGRGYQFASALEGALKIKEISYMHSEGVLAGELKHGVLALVDEELPIIAFGTRDSLFPKVVSSIEQVTARKGHPIIICNENDEVWNKKKSENDKFITLEVPTTVDCLQGLINIIPLQLMSYWLASNKGIDVDFPRNLAKSVTVE

SEQ ID NO: 28 - protein, 724 aa, Zygosaccharomyces parabailii strain ATCC 60483
MCGIFGYCNYLVERSRGDIIDTLVEGLERLEYRGYDSTGIAIDGDQPESTLLYKQIGKVAALKEEISHDAPRRDVSFVSHCGIAHTRWATHGEPKQVNCHPQKSDPNCEFTVVHNGIITNFRDLKTLLINKGYTFESDTDTECIAKLFKHLYDENLSNGHELDFHVLTKLVLLELEGSYGLLCRSSHYPEEVIATRKGSPLLIGVKSEKKLKVDFVDVEFPDDNAGKPDTPLVSRSGTPNGSARSQTTNGFSGLRRTRPYDSNNLLPITPNEFNLRHSQSRAFLSEDGKPNPVEFFVSSDAASVIKHTKKVLFLEDDDIAHIYDGELHIHRSRREVGASMTRSIQTLEMELAQIMKGPYTHFMQKEIFEQPDSTFNTMRGRIDFDSSTVMLGGLQAWLPAVRRARRLIMIACGTSYHSCLATRAIFEELSEIPVSVELASDFLDRKCPVFRDDVCCFVSQSGETADTMLALNYCLERGALTVGIVNSVGSSISRVTHCGVHINAGPEIGVASTKAYTSQYIALVMFALSLSDDRVSKMERRREIMNGLRNIPDQIREVLSLEPRIKELCQQDLEEHKSLLLLGRGYQFATALEGALKIKEISYMHSEGVLAGELKHGVLALVDEQLPIIAFATRDSLFPKVVSTIEQVTARKGHPIIICNKNDDVWAKKAETKQLVTLEVPQTVDCLQGLLNIIPLQLISYWLAVNRGIDVDFPRNLAKSVTVE

SEQ ID NO: 29 - protein, 724 aa, Zygosaccharomyces bailii ISA1307
MCGIFGYCNYLVERSRGDIIDTLVEGLERLEYRGYDSTGIAIDGDQPESTLLYKQIGKVAALKEEISHDAPRRDVSFVSHCGIAHTRWATHGEPKQVNCHPQKSDPNCEFTVVHNGIITNFRDLKTLLINKGYTFESDTDTECIAKLFKHLYDENLSNGHELDFHVLTKLVLLELEGSYGLLCRSSHYPEEVIATRKGSPLLIGVKSEKKLKVDFVDVEFPDDNAGKPDTPLVSRSGTPNGSARSQTTNGFSGLRRTRPYDSNNLLPITPNEFNLRHSQSRAFLSEDGKPNPVEFFVSSDAASVIKHTKKVLFLEDDDIAHIYDGELHIHRSRREVGASMTRSIQTLEMELAQIMKGPYTHFMQKEIFEQPDSTFNTMRGRIDFDSGTVMLGGLQAWLPAVRRARRLIMIACGTSYHSCLATRAIFEELSEIPVSVELASDFLDRKCPVFRDDVCCFVSQSGETADTMLALNYCLERGALTVGIVNSVGSSISRVTHCGVHINAGPEIGVASTKAYTSQYIALVMFALSLSDDRVSKMERRREIMNGLRNIPDQIREVLSLEPRIKELCQQDLEEHKSLLLLGRGYQFATALEGALKIKEISYMHSEGVLAGELKHGVLALVDEQLPIIAFATRDSLFPKVVSTIEQVTARKGHPIIICNKNDDVWAKKAETKQLVTLEVPQTVDCLQGLLNIIPLQLISYWLAVNRGIDVDFPRNLAKSVTVE

SEQ ID NO: 30 - protein, 724 aa, Zygosaccharomyces parabailii strain ATCC 60483
MCGIFGYCNYLVERSRGDIIDTLVEGLERLEYRGYDSTGIAIDGDQPESTLLYKQIGKVAALKEEISHDAPRRDVSFVSHCGIAHTRWATHGEPKQVNCHPQKSDPSCEFTVVHNGIITNFRDLKTLLINKGYTFESDTDTECIAKLFKHLYDENLSNGHELDFHVLTKLVLLELEGSYGLLCRSSHYPEEVIATRKGSPLLIGVKSEKKLKVDFVDVEFPDDNAGKPDTPLVSRSGTPNGSARSQTTNGFSGLRRTRPYDSNNLLPITPNEFNLRHSQSRAFLSEDGKPNPVEFFVSSDAASVIKHTKKVLFLEDDDIAHIYDGELHIHRSRREVGASMTRSIQTLEMELAQIMKGPYTHFMQKEIFEQPDSTFNTMRGRIDFDSGTVMLGGLQAWLPAVRRARRLIMIACGTSYHSCLATRAIFEELSEIPVSVELASDFLDRKCPVFRDDVCCFVSQSGETADTMLALNYCLERGALTVGIVNSVGSSISRVTHCGVHINAGPEIGVASTKAYTSQYIALVMFALSLSDDRVSKMERRREIMNGLRNIPDQIREVLSLEPRIKELCQQDLEEHKSLLLLGRGYQFATALEGALKIKEISYMHSEGVLAGELKHGVLALVDEQLPIIAFATRDSLFPKVVSTIEQVTARKGHPVIICNKNDDVWAKKAETKQLVTLEVPQTVDCLQGLLNIIPLQLISYWLAVNRGIDVDFPRNLAKSVTVE

SEQ ID NO: 31 - protein, 721 aa, Zygosaccharomyces mellis Ca-7
MCGIFGYCNYLVERSRGEIIDTLVEGLQRLEYRGYDSTGIAIDGDTPDKTLLYKQIGKVDALKQEIARDAPERDIAFASHCGIAHTRWATHGEPRQINCHPQRSDSQNAFTVVHNGIITNFRELRTLLANRGYTFESDTDTECIAILFKHLYDENLSNGHELDFHELTKLVLTELEGSYGLLCKSVHYPEEVIATRKGSPLLIGVKSDKKLKVDFVDIEFPDDTAGKPDTPLGSKNATPNASSRNLLNGNRRTRPFDGNNLLPLTANEFNLRHSQSRAFLSEDGTPNPVEFFVSSDAASVIKHTKKVLFLEDDDIAHIYDGELHIHRSRRPEVGASMTRSIQTLEMELAQIMKGPYKHFMQKEIFEQPDSALNTMRGRIDFENGSIMLGGLKAWLPAVRRARRLIMIACGTSYHSCLATRAMFEELSEIPVSVELASDFLDRKCPVFRDDVCCFVSQSGETADTMLALNYCLERGALTLGIVNSVGSSISRATHCGVHINAGPEIGVASTKAYTSQYIALVMFALSLSDDRVSKIERRKDIIRGLHNIPDQIREVLQLESRIKELCQNDLKDHKSLLVLGRGYQFASALEGALKIKEISYMHSEGVLAGELKHGVLALVDEQLPIIAFGTRDSLFPKVVSSIEQVTARKGRPIIICNTNDEVWAKKAETNKLLTLEVPQTVDCLQGLLNIIPLQLVSYWLAVNRGIDVDFPRNLAKSVTVE

SEQ ID NO: 32 - protein, 716 aa, Eremothecium cymbalariae DBVPG#7215
MCGIFGYCNFLVEKSRGEIIETLVEGLQRLEYRGYDSTGIAIDGDEADETFIFKQIGKVLALQKEIDDMQPRRDVRFVSHCGIAHTRWATHGQPRQVNCHPQRSDDGNEFVIVHNGIITNFRELKTLLVNKGYEFESDTDTECIAKLFKHLYHTNLKNGHELDFHELSKQVLLELEGSYGLLCRSSRYPGEVIATRKGSPLLIGVKSERRLKVDFVDVEFPDTNKAAEVPLSSNNGDSNNRHNDHHKANGDGHANLFPLTTGNESGLRHSQSRAFLSEDGVPNPVEFFLSSDAASVIKHTKKVLFLEDDDIAHIYDGELHIHRSRREVGASATRSIQTLEMELAQIMKGPYKHFMQKEIFEQPESTLNTMRGRIDFENNTVMLGGLKSWLPAIRNARRLTMIACGTSYHSCLATRAIFEELSEIPVSIELASDFMDRRCPVFRDDVCIFVSQSGETADTMLALNYCIERGALTVGIVNSVGSSISRVTHCGVHINAGPEIGVASTKAYTSQYIALVMLALSLSDDRVSRIERRKEIIQGLKKIPDQIKEVLQLERQIKQLCENELCNQKSLLLLGRGYQFASALEGALKIKEISYMHSEGVLAGELKHGVLALVDEHLPIIAFGTRDSLFPKVVSSIEQVTARKGRPIIICNENDDVWTKKAASSNLVTLEVPLTVDCLQGLLNIIPLQLTAYWLAVNNGIDVDFPRNLAKSVTVE

SEQ ID NO: 33 - protein, 704 aa, Kluyveromyces dobzhanskii CBS 2104
MCGIFGYCNFLVEKSRGEIIDTLVEGLQRLEYRGYDSTGIAIDGDREDETSIYKQIGKVSALKKEIEAHKPNRDAHFVTHCGIAHTRWATHGEPKQVNCHPHRSDPTGQFVVVHNGIITNFRDLRQLLSNKGYKFESDTDTECIAKLFKHIYDTNMKNGHDPDFHELTMQVLQELEGSYGLLCKSTHYPNEIIATRKGSPLLIGVKSEKKLKVDFVDVEFPDSEQLPDVPVNNECNLLNEAERSLLPIAGNESTLRRSQSRAFLSEDGSPSPIEFFLSSDAASIVKHTKKVLFLEDDDIAHIYDGELHIHRSTKKLTESATRSIQTLEMELAQIMKGPYKHFMQKEIYEQPESTFNTMRGRIDFEHNQVNLGGLKSRLPAIRKARRLIMIACGTSYHSCLATRSIFEELTEIPVSVELASDFLDRKCPIFRDDVCIFVSQSGETADTMLSLNYCLERGSLTLGIVNSVGSSISRSTHCGVHINAGPEIGVASTKAYTSQYIALVMVALSLSDDRLSKVERRNEIIQGLKLIPGQIKAVLNLEPRVKDLCAKELKNQKSLLLLGRGYQFATALEGALKIKEISYMHSEGVLAGELKHGVLALVDENLPIIAFGTRDSLFPKVLSSIEQVTARKGRPIVICNENDEVWQSKVKTHNLVTLEVPHTVDCLQGLLNVIPLQLISYWLAVNNGIDVDFPRNLAKSVTVE

SEQ ID NO: 34 - protein, 704 aa, Kluyveromyces lactis strain NRRL Y-1140
MCGIFGYCNFLVEKSRGEIIDTLVEGLQRLEYRGYDSTGIAIDGDREDETVIYKQIGKVSALKKEIEAHKPNRDAHFVTHCGIAHTRWATHGEPKQVNCHPHRSDPSGTFVVVHNGIITNFRDLRQLLTNKGYKFESDTDTECIAKLFKHIYDTNLKNGHDPDFHELTMQVLQELEGSYGLLCKSTHYPNEIIATRKGSPLLIGVKSEKKLKVDFVDVEFPDSEQLPDLPVNNECNLLNDADRSLLPLAGDESTLRRSQSRAFLSEDGSPSPIEFFLSSDAASIVKHTKKVLFLEDDDIAHIYDGELHIHRANKKLTESATRSIQTLEMELAQIMKGPYQHFMQKEIYEQPESTLNTMRGRIDFEHNLVNLGGLKSRLSAIRKARRLIMIACGTSYHSCLATRAIFEELTEIPVSVELASDFLDRKCPIFRDDVCIFVSQSGETADTMLSLNYCLERGALTLGIVNSVGSSISRSTHCGVHINAGPEIGVASTKAYTSQYIALVMVALSLSDDRVSKIERRNEIIQGLKLIPGQIKEVLNVEPRIKELCAKELKDQKSLLLLGRGYQFASALEGALKIKEISYMHSEGVLAGELKHGVLALVDEDLPIIAFGTRDSLFPKVLSSIEQVIARKGHPIIICNENDEVWQNKAKANNLITLEVPQTVDCLQGLLNIIPLQLTSYWLAVNNGIDVDFPRNLAKSVTVE

SEQ ID NO: 35 - protein, 703 aa, Eremothecium sinecaudum strain ATCC 58844
MCGIFGYCNYLVEKTRGDVIDTLVEGLQRLEYRGYDSTGIAIDGDNAGETFIFKQIGKVSALQKEIDVCKPNRDVSFVSHCAIAHTRWATHGQPRKVNCHPQRSDENNEFVVVHNGIITNFRELKTLLVNKGFKFESDTDTECIAKLFKHLYDTNMENGHEVDFHELSKQVLLELEGSYGLLCRSSRYPNEVIATRKGSPLLIGVKSERKLKVDFVDVEFPDNEDKREVPLAARNSHHKRNENLLPITTTESHLRHSQSRAFLSEDGAPNPVEFYLSSDAASVIKHTKKVLFLEDDDIAHIYDGELHIHRSRREVGASATRSIQTLEMELAQIMKGPYKHFMQKEIFEQPESTLNTMRGRIDFENNTVMLGGLKSWLPAIRNARRLTMIACGTSYHSCMAARAIFEELSEILVSIELASDFLDRRCPVFRDDVCVFVSQSGETADTMLALNYCIERGALTVGIVNSVGSSISRVTHCGVHINAGPEIGVASTKAYTSQYIALVMLALSLSDDRVSRIERRKEIIQGLKKIPEQIKQVLQLEAPIKKLCETELVNQKSLLLLGRGYQFASALEGALKIKEISYMHSEGVLAGELKHGVLALVDEHLPIIAFGTRDSLFPKVVSSIEQIIARKGRPIIICNENDEVWEKKATTAEVITLEVPQTVDCLQGLLNIIPLQLTAYWLAVNNGIDVDFPRNLAKSVTVE

SEQ ID NO: 36 - protein, 704 aa, Kluyveromyces lactis strain CBS 2105
MCGIFGYCNFLVEKSRGEIIDTLVEGLQRLEYRGYDSTGIAIDGDREDETVIYKQIGKVSALKKEIEAHKPNRDAHFVTHCGIAHTRWATHGEPKQVNCHPHRSDPSGTFVVVHNGIITNFRDLRQLLTNKGYKFESDTDTECIAKLFKHIYDTNLKNGHDPDFHELTMQVLQELEGSYGLLCKSTHYPNEIIATRKGSPLLIGVKSEKKLKVDFVDVEFPDSEQLPDLPVNNECNLLNDADRSLLPLAGDESTLRRSQSRAFLSEDGSPSPIEFFLSSDAASIVKHTKKVLFLEDDDIAHIYDGELHIHRANKKLTESATRSIQTLEMELAQIMKGPYQHFMQKEIYEQPESTLNTMRGRIDFEHNLVNLGGLKSRLSAIRKARRLIMIACGTSYHSCLATRAIFEELTEIPVSVELASDFLDRKCPIFRDDVCIFVSQSGETADTMLSLNYCLERGALTLGIVNSVGSSISRSTHCGVHINAGPEIGVASTKAYTSQYIALVMVALSLSDDRVSKIERRNEIIQGLKLIPGQIKEVLNVEPRIKELCAKELKDQKSLLLLGRGYQFASALEGALKIKEISYMHSEGVLAGELKHGVLALVDEDLPIIAFGTRDSLFPKVLSSIEQVIARKGHPIVICNENDEVWQNKAKANNLITLEVPQTVDCLQGLLNIIPLQLTSYWLAVNNGIDVDFPRNLAKSVTVE

SEQ ID NO: 37 - protein, 706 aa, Kluyveromyces marxianus DMKU3-1042
MCGIFGYCNFLVEKSRGEIVDTLVEGLQRLEYRGYDSTGIAIDGDDEDETLIFKQIGKVSALQKEIEKHNPNRDVHFVTHCGIAHTRWATHGEPKQVNCHPHRSDVTCQFVVVHNGIITNYRDLRQLLTNKGYKFESDTDTECIAKLFMHIYQTNLKNGHDPDFHELTKQVLQELEGSYGLLCKSTHYPNEIIATRKGSPLLIGVKSEKKMKVDFVDVEFPDSEQMPDVPVNNECNLLNDAERSLLPLAGNESTLRRSQSRAFLSEDGSPSPIEFFLSSDAASIVKHTKKVLFLEDDDIAHIYDGELHIHRATNKLNESATRSIQTLEMELAQIMKGPYKHFMQKEIYEQPESTLNTMRGRIDFETNTVTLGGLTSRLPAIRRARRLIMIACGTSYHSCLAARAIFEELTEIPVSVELASDFLDRRAPIFRDDVCIFVSQSGETADTMLSLNYCLERGALTLGIVNSVGSSISRGTHCGVHINAGPEIGVASTKAYTSQFIALVMVALSLSDDRLSKLERRKEIIQGLKLIPEQIKQVLKLESQVKDLCSNELKDQKSLLLLGRGYQFASALEGALKIKEISYMHSEGVLAGELKHGVLALVDENLPIIAFGTRDSLFPKVLSSIEQVTARKGHPIIICNENDEVWEAKKKNSNGSLVTLEVPRTVDCLQGLLNIIPLQLTSYWLAVNNGIDVDFPRNLAKSVTVE

SEQ ID NO: 38 - protein, 723 aa, Tetrapisispora blattae CBS 6284
MCGIFGYCNYLVEKTRGEIIDILVEGLGRLEYRGYDSTGIAINGDKRNSIDVYRAIGKVSALKQEIDNINPNRDVTYASHSGIAHTRWATHGQPKQTNCHPQRSDPRNLFTIIHNGIITNFRELKTLLVNKGYTFESDTDTECIAKLFLHLYQTNIKMGNDIDFHELTKLVLMELEGSYGLLCKSVYFPGEIIATRKGSPLLIGVKSENKLKVDFVDVEFPNENAIQTETPLSNECKTDNNDELNSSNYPTVDKKEFHEKAGLVPITANEFNLRHSQSRAFLSEDGSPIPIEFFISSDAASVIKYTKKVLFLEDDDIAHIYDGELHIHRSRKEVGKSMIRSIQTLELELAQIMKGPYEHFMQKEIYEQPVSTYNTMRGRLDFDNNLVNLGGLQQFISTIRRARRLIMIACGTSYHSCLATRAIFEELSDIPVNVDLASDFLDRKCPIFRDDVCVFVSQSGETADTMFALNYAIDRGALTVGIVNSVGSSISRATNCGVHINAGPEIGVASTKAYTSQYIALVMFAVLLGEDRISKNERRREIIQGLKLIPEQIKQVLKLEPKIKQLCENELSDQKSLLLLGRGYQFASALEGALKIKEISYMHSEGVLAGELKHGVLALVDEELPIIAFGTRDYLFPKVVSSIEQVTARKGHPIIICNENDEVWSKKKLENDRLITLEVPTTVDCLQGLINIIPLQLMSYWLASNRGIDVDFPRNLAKSVTVE

SEQ ID NO: 39 - protein, 717 aa, Artificial sequence
  synthetic sequence
MCGIFGYCNYLLERSRGEIIDTLVDGLQRLEYRGYDSTGIAIDGDEADSTFIYKQIGKVSALKEEITKQNPNRDVTFVSHCGIAHTRWATHGRPEHVNCHPQRSDPEDQFVVVHNGIITNFRELKTLLINKGYKFESDTDTECIAKLYLHLYNTNLRNGHDLDFHELTKLVLLELEGSYGLLCKSCHYPNEVIATRKGSPLLIGVKSEKKLKVDFVDVEFPEENAGQPEIPLKSNNKSFGLGPKKAREFEAGSQNANLLPIAANEFNLRHSQSRAFLSEDGSPTPVEFFVSSDAASVVKHTKKVLFLEDDDLAHIYDGELHIHRSRREVGASMTRSIQTLEMVLAQIMKGPYDHFMQKEIYEQPESTFNTMRGRIDYENNKVILGGLKAWLPVVRRARRLIMIACGTSYHSCLATRAIFEELSDIPVSVELASDFLDRKCPVFRDDVCVFVSQSGETADTMLALNYCLERGALTVGIVNSVGSSISRVTHCGVHINAGPEIGVASTKAYTSQYIALVMFALSLSDDRVSKIDRRIEIIQGLKLIPGQIKQVLKLEPRIKKLCATELKDQKSLLLLGRGYQFAAALEGALKIKEISYMHSEGVLAGELKHGVLALVDENLPIIAFGTRDSLFPKVVSSIEQVTARKGHPIIICNENDEVWAQKSKSIDLQTLEVPQTVDCLQGLINIIPLQLMSYWLAVNKGIDVDFPRNLAKSVTVE

SEQ ID NO: 40 - protein, 717 aa, Artificial sequence
  synthetic sequence
MCGIFGYCNYLVERSRGEIIDTLVDGLQRLEYRGYDSTGIAIDGDEADSTFIYKQIGKVSALKEEITKQNPNRDVTFVSHCGIAHTRWATHGRPEHVNCHPQRSDPEDQFVVVHNGIITNFRELKTLLINKGYKFESDTDTECIAKLYLHLYNTNLRNGHDLDFHELTKLVLLELEGSYGLLCKSCHYPNEVIATRKGSPLLIGVKSEKKLKVDFVDVEFPEENAGQPEIPLKSNNKSFGLGPKKAREFEAGSQNANLLPIAANEFNLRHSQSRAFLSEDGSPTPVEFFVSSDAASVVKHTKKVLFLEDDDLAHIYDGELHIHRSRREVGASMTRSIQTLEMVLAQIMKGPYDHFMQKEIYEQPESTFNTMRGRIDYENNKVILGGLKAWLPVVRRARRLIMIACGTSYHSCLATRAIFEELSDIPVSVELASDFLDRKCPVFRDDVCVFVSQSGETADTMLALNYCLERGALTVGIVNSVGSSISRVTHCGVHINAGPEIGVASTKAYTSQYIALVMFALSLSDDRVSKIDRRIEIIQGLKLIPGQIKQVLKLEPRIKKLCATELKDQKSLLLLGRGYQFAAALEGALKIKEISYMHSEGVLAGELKHGVLALVDENLPIIAFGTRDSLFPKVVSSIEQVTARKGHPIIICNENDEVWAQKSKSIDLQTLEVPQTVDCLQGLINIIPLQLMSYWLAVNKGIDVDFPRNLAKSVTVE

SEQ ID NO: 41 - protein, 717 aa, Artificial sequence
  synthetic sequence
MCGIFGYCNYLLERSRGEIIDTLVDGLQRLEYRGYDSTGIAIDGDEADSTFIYKQIGKVSALKEEITKQNPNRDVTFVSHCGIAHTRWATHGRPEHVNCHPQRSDPEDQFVVVHNGIITNFRELKTLLINKGYKFESDTDTECIAKLYLHLYNTNLRNGHDLDFHELTKLVLLELEGSYGLLCKSCHYPNEVIATRKGSPLLIGVKSEKKLKVDFVDVEFPEENAGQPEIPLKSNNKSFGLGPKKAREFEAGSQNANLLPIAANEFNLRHSQSRAFLSEDGSPTPVEFFVSSDAASVVKHTKKVLFLEDDDLAHIYDGELHIHRSRREVGASMTRSIQTLEMELAQIMKGPYDHFMQKEIYEQPESTFNTMRGRIDYENNKVILGGLKAWLPVVRRARRLIMIACGTSYHSCLATRAIFEELSDIPVSVELASDFLDRKCPVFRDDVCVFVSQSGETADTMLALNYCLERGALTVGIVNSVGSSISRVTHCGVHINAGPEIGVASTKAYTSQYIALVMFALSLSDDRVSKIDRRIEIIQGLKLIPGQIKQVLKLEPRIKKLCATELKDQKSLLLLGRGYQFAAALEGALKIKEISYMHSEGVLAGELKHGVLALVDENLPIIAFGTRDSLFPKVVSSIEQVTARKGHPIIICNENDEVWAQKSKSIDLQTLEVPQTVDCLQGLINIIPLQLMSYWLAVNKGIDVDFPRNLAKSVTVE

SEQ ID NO: 42 - protein, 717 aa, Artificial sequence
  synthetic sequence
MCGIFGYCNYLVERSRGEIIDTLVDGLQRLEYRGYDSTGIAIDGDEADSTFIYKQIGKVSALKEEITKQNPNRDVTFVSHCGIAHTRWATHGRPEHVNCHPQRSDPEDQFVVVHNGIITNFRELKTLLINKGYKFESDTDTECIAKLYLHLYNTNLRNGHDLDFHELTKLVLLELEGSYGLLCKSCHYPNEVIATRKGSPLLIGVKSEKKLKVDFVDVEFPEENAGQPEIPLKSNNKSFGLGPKKAREFEAGSQNANLLPIAANEFNLRHSQSRAFLSEDGSPTPVEFFVSSDAASVVKHTKKVLFLEDDDLAHIYDGELHIHRSRREVGASMTRSIQTLEMELAQIMKGPYDHFMQKEIYEQPESTFNTMRGRIDYENNKVILGGLKAWLPVVRRARRLIMIACGTSYHSCLATRAIFEELSDIPVSVELASDFLDRKCPVFRDDVCVFVSQSGETADTMLALNYCLERGALTVGIVNSVGSSISRVTHCGVHINAGPEIGVASTKAYTSQYIALVMFALSLSDDRVSKIDRRIEIIQGLKLIPGQIKQVLKLEPRIKKLCATELKDQKSLLLLGRGYQFAAALEGALKIKEISYMHSEGVLAGELKHGVLALVDENLPIIAFGTRDSLFPKVVSSIEQVTARKGHPIIICNENDEVWAQKSKSIDLQTLEVPQTVDCLQGLINIIPLQLMSYWLAVNKGIDVDFPRNLAKSVTVE

SEQ ID NO: 43 - protein, 717 aa, Artificial sequence
  synthetic sequence
MCGIFGYCNYLLERSRGEIIDTLVDGLQRLEYRGYDSTGIAIDGDEADSTFIYKQIGKVSALKEEITKQNPNRDVTFVSHCGIAHTRWATHGRPEQVNCHPQRSDPEDQFVVVHNGIITNFRELKTLLINKGYKFESDTDTECIAKLYLHLYNTNLRNGHDLDFHELTKLVLLELEGSYGLLCKSCHYPNEVIATRKGSPLLIGVKSEKKLKVDFVDVEFPEENAGQPEIPLKSNNKSFGLGPKKAREFEAGSQNANLLPIAANEFNLRHSQSRAFLSEDGSPTPVEFFVSSDAASVVKHTKKVLFLEDDDLAHIYDGELHIHRSRREVGASMTRSIQTLEMVLAQIMKGPYDHFMQKEIYEQPESTFNTMRGRIDYENNKVILGGLKAWLPVVRRARRLIMIACGTSYHSCLATRAIFEELSDIPVSVELASDFLDRKCPVFRDDVCVFVSQSGETADTMLALNYCLERGALTVGIVNSVGSSISRVTHCGVHINAGPEIGVASTKAYTSQYIALVMFALSLSDDRVSKIDRRIEIIQGLKLIPGQIKQVLKLEPRIKKLCATELKDQKSLLLLGRGYQFAAALEGALKIKEISYMHSEGVLAGELKHGVLALVDENLPIIAFGTRDSLFPKVVSSIEQVTARKGHPIIICNENDEVWAQKSKSIDLQTLEVPQTVDCLQGLINIIPLQLMSYWLAVNKGIDVDFPRNLAKSVTVE

SEQ ID NO: 44 - protein, 717 aa, Artificial sequence
  synthetic sequence
MCGIFGYCNYLLERSRGEIIDTLVDGLQRLEYRGYDSTGIAIDGDEADSTFIYKQIGKVSALKEEITKQNPNRDVTFVSHCGIAHTRWATHGRPEHVNCHPQRSDPEDQFVVVHNGIITNFRELKTLLINKGYKFESDTDTECIAKLYLHLYNTNLQNGHDLDFHELTKLVLLELEGSYGLLCKSCHYPNEVIATRKGSPLLIGVKSEKKLKVDFVDVEFPEENAGQPEIPLKSNNKSFGLGPKKAREFEAGSQNANLLPIAANEFNLRHSQSRAFLSEDGSPTPVEFFVSSDAASVVKHTKKVLFLEDDDLAHIYDGELHIHRSRREVGASMTRSIQTLEMVLAQIMKGPYDHFMQKEIYEQPESTFNTMRGRIDYENNKVILGGLKAWLPVVRRARRLIMIACGTSYHSCLATRAIFEELSDIPVSVELASDFLDRKCPVFRDDVCVFVSQSGETADTMLALNYCLERGALTVGIVNSVGSSISRVTHCGVHINAGPEIGVASTKAYTSQYIALVMFALSLSDDRVSKIDRRIEIIQGLKLIPGQIKQVLKLEPRIKKLCATELKDQKSLLLLGRGYQFAAALEGALKIKEISYMHSEGVLAGELKHGVLALVDENLPIIAFGTRDSLFPKVVSSIEQVTARKGHPIIICNENDEVWAQKSKSIDLQTLEVPQTVDCLQGLINIIPLQLMSYWLAVNKGIDVDFPRNLAKSVTVE

SEQ ID NO: 45 - protein, 717 aa, Artificial sequence
  synthetic sequence
MCGIFGYCNYLVERSRGEIIDTLVDGLQRLEYRGYDSTGIAIDGDEADSTFIYKQIGKVSALKEEITKQNPNRDVTFVSHCGIAHTRWATHGRPEHVNCHPQRSDPEDQFVVVHNGIITNFRELKTLLINKGYKFESDTDTECIAKLYLHLYNTNLQNGHDLDFHELTKLVLLELEGSYGLLCKSCHYPNEVIATRKGSPLLIGVKSEKKLKVDFVDVEFPEENAGQPEIPLKSNNKSFGLGPKKAREFEAGSQNANLLPIAANEFNLRHSQSRAFLSEDGSPTPVEFFVSSDAASVVKHTKKVLFLEDDDLAHIYDGELHIHRSRREVGASMTRSIQTLEMVLAQIMKGPYDHFMQKEIYEQPESTFNTMRGRIDYENNKVILGGLKAWLPVVRRARRLIMIACGTSYHSCLATRAIFEELSDIPVSVELASDFLDRKCPVFRDDVCVFVSQSGETADTMLALNYCLERGALTVGIVNSVGSSISRVTHCGVHINAGPEIGVASTKAYTSQYIALVMFALSLSDDRVSKIDRRIEIIQGLKLIPGQIKQVLKLEPRIKKLCATELKDQKSLLLLGRGYQFAAALEGALKIKEISYMHSEGVLAGELKHGVLALVDENLPIIAFGTRDSLFPKVVSSIEQVTARKGHPIIICNENDEVWAQKSKSIDLQTLEVPQTVDCLQGLINIIPLQLMSYWLAVNKGIDVDFPRNLAKSVTVE

SEQ ID NO: 46 - protein, 717 aa, Artificial sequence
  synthetic sequence
MCGIFGYCNYLVERSRGEIIDTLVDGLQRLEYRGYDSTGIAIDGDEADSTFIYKQIGKVSALKEEITKQNPNRDVTFVSHCGIAHTRWATHGRPEQVNCHPQRSDPEDQFVVVHNGIITNFRELKTLLINKGYKFESDTDTECIAKLYLHLYNTNLRNGHDLDFHELTKLVLLELEGSYGLLCKSCHYPNEVIATRKGSPLLIGVKSEKKLKVDFVDVEFPEENAGQPEIPLKSNNKSFGLGPKKAREFEAGSQNANLLPIAANEFNLRHSQSRAFLSEDGSPTPVEFFVSSDAASVVKHTKKVLFLEDDDLAHIYDGELHIHRSRREVGASMTRSIQTLEMVLAQIMKGPYDHFMQKEIYEQPESTFNTMRGRIDYENNKVILGGLKAWLPVVRRARRLIMIACGTSYHSCLATRAIFEELSDIPVSVELASDFLDRKCPVFRDDVCVFVSQSGETADTMLALNYCLERGALTVGIVNSVGSSISRVTHCGVHINAGPEIGVASTKAYTSQYIALVMFALSLSDDRVSKIDRRIEIIQGLKLIPGQIKQVLKLEPRIKKLCATELKDQKSLLLLGRGYQFAAALEGALKIKEISYMHSEGVLAGELKHGVLALVDENLPIIAFGTRDSLFPKVVSSIEQVTARKGHPIIICNENDEVWAQKSKSIDLQTLEVPQTVDCLQGLINIIPLQLMSYWLAVNKGIDVDFPRNLAKSVTVE

SEQ ID NO: 47 - protein, 717 aa, Artificial sequence
  synthetic sequence
MCGIFGYCNYLLERSRGEIIDTLVDGLQRLEYRGYDSTGIAIDGDEADSTFIYKQIGKVSALKEEITKQNPNRDVTFVSHCGIAHTRWATHGRPEHVNCHPQRSDPEDQFVVVHNGIITNFRELKTLLINKGYKFESDTDTECIAKLYLHLYNTNLQNGHDLDFHELTKLVLLELEGSYGLLCKSCHYPNEVIATRKGSPLLIGVKSEKKLKVDFVDVEFPEENAGQPEIPLKSNNKSFGLGPKKAREFEAGSQNANLLPIAANEFNLRHSQSRAFLSEDGSPTPVEFFVSSDAASVVKHTKKVLFLEDDDLAHIYDGELHIHRSRREVGASMTRSIQTLEMELAQIMKGPYDHFMQKEIYEQPESTFNTMRGRIDYENNKVILGGLKAWLPVVRRARRLIMIACGTSYHSCLATRAIFEELSDIPVSVELASDFLDRKCPVFRDDVCVFVSQSGETADTMLALNYCLERGALTVGIVNSVGSSISRVTHCGVHINAGPEIGVASTKAYTSQYIALVMFALSLSDDRVSKIDRRIEIIQGLKLIPGQIKQVLKLEPRIKKLCATELKDQKSLLLLGRGYQFAAALEGALKIKEISYMHSEGVLAGELKHGVLALVDENLPIIAFGTRDSLFPKVVSSIEQVTARKGHPIIICNENDEVWAQKSKSIDLQTLEVPQTVDCLQGLINIIPLQLMSYWLAVNKGIDVDFPRNLAKSVTVE

SEQ ID NO: 48 - protein, 717 aa, Artificial sequence
  synthetic sequence
MCGIFGYCNYLLERSRGEIIDTLVDGLQRLEYRGYDSTGIAIDGDEADSTFIYKQIGKVSALKEEITKQNPNRDVTFVSHCGIAHTRWATHGRPEQVNCHPQRSDPEDQFVVVHNGIITNFRELKTLLINKGYKFESDTDTECIAKLYLHLYNTNLRNGHDLDFHELTKLVLLELEGSYGLLCKSCHYPNEVIATRKGSPLLIGVKSEKKLKVDFVDVEFPEENAGQPEIPLKSNNKSFGLGPKKAREFEAGSQNANLLPIAANEFNLRHSQSRAFLSEDGSPTPVEFFVSSDAASVVKHTKKVLFLEDDDLAHIYDGELHIHRSRREVGASMTRSIQTLEMELAQIMKGPYDHFMQKEIYEQPESTFNTMRGRIDYENNKVILGGLKAWLPVVRRARRLIMIACGTSYHSCLATRAIFEELSDIPVSVELASDFLDRKCPVFRDDVCVFVSQSGETADTMLALNYCLERGALTVGIVNSVGSSISRVTHCGVHINAGPEIGVASTKAYTSQYIALVMFALSLSDDRVSKIDRRIEIIQGLKLIPGQIKQVLKLEPRIKKLCATELKDQKSLLLLGRGYQFAAALEGALKIKEISYMHSEGVLAGELKHGVLALVDENLPIIAFGTRDSLFPKVVSSIEQVTARKGHPIIICNENDEVWAQKSKSIDLQTLEVPQTVDCLQGLINIIPLQLMSYWLAVNKGIDVDFPRNLAKSVTVE

SEQ ID NO: 49 - protein, 717 aa, Artificial sequence
  synthetic sequence
MCGIFGYCNYLLERSRGEIIDTLVDGLQRLEYRGYDSTGIAIDGDEADSTFIYKQIGKVSALKEEITKQNPNRDVTFVSHCGIAHTRWATHGRPEQVNCHPQRSDPEDQFVVVHNGIITNFRELKTLLINKGYKFESDTDTECIAKLYLHLYNTNLQNGHDLDFHELTKLVLLELEGSYGLLCKSCHYPNEVIATRKGSPLLIGVKSEKKLKVDFVDVEFPEENAGQPEIPLKSNNKSFGLGPKKAREFEAGSQNANLLPIAANEFNLRHSQSRAFLSEDGSPTPVEFFVSSDAASVVKHTKKVLFLEDDDLAHIYDGELHIHRSRREVGASMTRSIQTLEMVLAQIMKGPYDHFMQKEIYEQPESTFNTMRGRIDYENNKVILGGLKAWLPVVRRARRLIMIACGTSYHSCLATRAIFEELSDIPVSVELASDFLDRKCPVFRDDVCVFVSQSGETADTMLALNYCLERGALTVGIVNSVGSSISRVTHCGVHINAGPEIGVASTKAYTSQYIALVMFALSLSDDRVSKIDRRIEIIQGLKLIPGQIKQVLKLEPRIKKLCATELKDQKSLLLLGRGYQFAAALEGALKIKEISYMHSEGVLAGELKHGVLALVDENLPIIAFGTRDSLFPKVVSSIEQVTARKGHPIIICNENDEVWAQKSKSIDLQTLEVPQTVDCLQGLINIIPLQLMSYWLAVNKGIDVDFPRNLAKSVTVE

SEQ ID NO: 50 - protein, 717 aa, Artificial sequence
  synthetic sequence
MCGIFGYCNYLLERSRGEIIDTLVDGLQRLEYRGYDSTGIAIDGDEADSTFIYKQIGKVSALKEEITKQNPNRDVTFVSHCGIAHTRWATHGRPEQVNCHPQRSDPEDQFVVVHNGIITNFRELKTLLINKGYKFESDTDTECIAKLYLHLYNTNLQNGHDLDFHELTKLVLLELEGSYGLLCKSCHYPNEVIATRKGSPLLIGVKSEKKLKVDFVDVEFPEENAGQPEIPLKSNNKSFGLGPKKAREFEAGSQNANLLPIAANEFNLRHSQSRAFLSEDGSPTPVEFFVSSDAASVVKHTKKVLFLEDDDLAHIYDGELHIHRSRREVGASMTRSIQTLEMELAQIMKGPYDHFMQKEIYEQPESTFNTMRGRIDYENNKVILGGLKAWLPVVRRARRLIMIACGTSYHSCLATRAIFEELSDIPVSVELASDFLDRKCPVFRDDVCVFVSQSGETADTMLALNYCLERGALTVGIVNSVGSSISRVTHCGVHINAGPEIGVASTKAYTSQYIALVMFALSLSDDRVSKIDRRIEIIQGLKLIPGQIKQVLKLEPRIKKLCATELKDQKSLLLLGRGYQFAAALEGALKIKEISYMHSEGVLAGELKHGVLALVDENLPIIAFGTRDSLFPKVVSSIEQVTARKGHPIIICNENDEVWAQKSKSIDLQTLEVPQTVDCLQGLINIIPLQLMSYWLAVNKGIDVDFPRNLAKSVTVE

SEQ ID NO: 51 - protein, 717 aa, Artificial sequence
  synthetic sequence
MCGIFGYCNYLVERSRGEIIDTLVDGLQRLEYRGYDSTGIAIDGDEADSTFIYKQIGKVSALKEEITKQNPNRDVTFVSHCGIAHTRWATHGRPEHVNCHPQRSDPEDQFVVVHNGIITNFRELKTLLINKGYKFESDTDTECIAKLYLHLYNTNLQNGHDLDFHELTKLVLLELEGSYGLLCKSCHYPNEVIATRKGSPLLIGVKSEKKLKVDFVDVEFPEENAGQPEIPLKSNNKSFGLGPKKAREFEAGSQNANLLPIAANEFNLRHSQSRAFLSEDGSPTPVEFFVSSDAASVVKHTKKVLFLEDDDLAHIYDGELHIHRSRREVGASMTRSIQTLEMELAQIMKGPYDHFMQKEIYEQPESTFNTMRGRIDYENNKVILGGLKAWLPVVRRARRLIMIACGTSYHSCLATRAIFEELSDIPVSVELASDFLDRKCPVFRDDVCVFVSQSGETADTMLALNYCLERGALTVGIVNSVGSSISRVTHCGVHINAGPEIGVASTKAYTSQYIALVMFALSLSDDRVSKIDRRIEIIQGLKLIPGQIKQVLKLEPRIKKLCATELKDQKSLLLLGRGYQFAAALEGALKIKEISYMHSEGVLAGELKHGVLALVDENLPIIAFGTRDSLFPKVVSSIEQVTARKGHPIIICNENDEVWAQKSKSIDLQTLEVPQTVDCLQGLINIIPLQLMSYWLAVNKGIDVDFPRNLAKSVTVE

SEQ ID NO: 52 - protein, 717 aa, Artificial sequence
  synthetic sequence
MCGIFGYCNYLVERSRGEIIDTLVDGLQRLEYRGYDSTGIAIDGDEADSTFIYKQIGKVSALKEEITKQNPNRDVTFVSHCGIAHTRWATHGRPEQVNCHPQRSDPEDQFVVVHNGIITNFRELKTLLINKGYKFESDTDTECIAKLYLHLYNTNLRNGHDLDFHELTKLVLLELEGSYGLLCKSCHYPNEVIATRKGSPLLIGVKSEKKLKVDFVDVEFPEENAGQPEIPLKSNNKSFGLGPKKAREFEAGSQNANLLPIAANEFNLRHSQSRAFLSEDGSPTPVEFFVSSDAASVVKHTKKVLFLEDDDLAHIYDGELHIHRSRREVGASMTRSIQTLEMELAQIMKGPYDHFMQKEIYEQPESTFNTMRGRIDYENNKVILGGLKAWLPVVRRARRLIMIACGTSYHSCLATRAIFEELSDIPVSVELASDFLDRKCPVFRDDVCVFVSQSGETADTMLALNYCLERGALTVGIVNSVGSSISRVTHCGVHINAGPEIGVASTKAYTSQYIALVMFALSLSDDRVSKIDRRIEIIQGLKLIPGQIKQVLKLEPRIKKLCATELKDQKSLLLLGRGYQFAAALEGALKIKEISYMHSEGVLAGELKHGVLALVDENLPIIAFGTRDSLFPKVVSSIEQVTARKGHPIIICNENDEVWAQKSKSIDLQTLEVPQTVDCLQGLINIIPLQLMSYWLAVNKGIDVDFPRNLAKSVTVE

SEQ ID NO: 53 - protein, 717 aa, Artificial sequence
  synthetic sequence
MCGIFGYCNYLVERSRGEIIDTLVDGLQRLEYRGYDSTGIAIDGDEADSTFIYKQIGKVSALKEEITKQNPNRDVTFVSHCGIAHTRWATHGRPEQVNCHPQRSDPEDQFVVVHNGIITNFRELKTLLINKGYKFESDTDTECIAKLYLHLYNTNLQNGHDLDFHELTKLVLLELEGSYGLLCKSCHYPNEVIATRKGSPLLIGVKSEKKLKVDFVDVEFPEENAGQPEIPLKSNNKSFGLGPKKAREFEAGSQNANLLPIAANEFNLRHSQSRAFLSEDGSPTPVEFFVSSDAASVVKHTKKVLFLEDDDLAHIYDGELHIHRSRREVGASMTRSIQTLEMVLAQIMKGPYDHFMQKEIYEQPESTFNTMRGRIDYENNKVILGGLKAWLPVVRRARRLIMIACGTSYHSCLATRAIFEELSDIPVSVELASDFLDRKCPVFRDDVCVFVSQSGETADTMLALNYCLERGALTVGIVNSVGSSISRVTHCGVHINAGPEIGVASTKAYTSQYIALVMFALSLSDDRVSKIDRRIEIIQGLKLIPGQIKQVLKLEPRIKKLCATELKDQKSLLLLGRGYQFAAALEGALKIKEISYMHSEGVLAGELKHGVLALVDENLPIIAFGTRDSLFPKVVSSIEQVTARKGHPIIICNENDEVWAQKSKSIDLQTLEVPQTVDCLQGLINIIPLQLMSYWLAVNKGIDVDFPRNLAKSVTVE

SEQ ID NO: 54 - protein, 348 aa, Neisseria meningitidis 35E
MPSEAFRRHRAYRENKLQPLVSVLICAYNVEKYFAQSLAAVVNQTWCNLDILIVDDGSTDGTLAIAQRFQEQDGRIKILAQAQNSGLIPSLNIGLDELAKSGMGEYIARTDADDIAAPDWIEKIVGEMEKDRSIIAMGAWLEVLSEEKDGNRLARHHEHGKIWKKPTRHEDIADFFPFGNPIHNNTMIMRRSVIDGGLRYNTERDWAEDYQFWYDVSKLGRLAYYPEALVKYRLHANQVSSKYSIRQHEIAQGIQKTARNDFLQSMGFKTRFDSLEYRQIKAVAYELLEKHLPEEDFERARRFLYQCFKRTDTLPAGVWLDFAANGRMRRLFTLRQYFGILHRLLKNR

SEQ ID NO: 55 - protein, 223 aa, Pasteurella multocida
MTNIAIIPARAGSKGIPDKNLQPVGGHSLIGRAILAAKNADVFDMIVVTSDGDNILREAEKYGALALKRPAELAQDNSRTIDAILHALESLNIREGTCTLLQPTSPLRDHLDIKNAMDMYVNGGVHSVVSACECEHHPYKAFALSKDHEVLPVREIADFEAARQTLPKMYRANGAIYINDIAQLLKEKYFFIPPLKFYLMPTYRSVDIDVKQDLELAEILSNK

SEQ ID NO: 56 - protein, 371 aa, Neisseria meningitidis
MGLKKACLTVLCLIVFCFGIFYTFDRVNQGERNAVSLLKEKLFNEEGEPVNLIFCYTILQMKVAERIMAQHPGERFYVVLMSENRNEKYDYYFNQIKDKAERAYFFHLPYGLNKSFNFIPTMAELKVKSMLLPKVKRIYLASLEKVSIAAFLSTYPDAEIKTFDDGTGNLIQSSSYLGDEFSVNGTIKRNFARMMIGDWSIAKTRNASDEHYTIFKGLKNIMDDGRRKMTYLPLFDASELKTGDETGGTVRILLGSPDKEMKEISEKAAKNFKIQYVAPHPRQTYGLSGVTTLNSPYVIEDYILREIKKNPHTRYEIYTFFSGAALTMKDFPNVHVYALKPASLPEDYWLKPVYALFTQSGIPILTFDDKN

SEQ ID NO: 57 - protein, 308 aa, Pasteurella multocida subsp. multocida str. Pm70
MNLIICCTPLQVLIAEKIIAKFPHMPFYGVMLSTVSNKKFDFYAKRLAQQCQGFFSMVQHKDRFNLLKEILYLKRTFSGKHFDQVFVANINDLQIQFLLSAIDFNLLNTFDDGTINIVPNSLFYQDDPATLQRKLINVLLGNKYSIQSLRALSHTHYTIYKGFKNIIERVEPIELVAADNSEKVTSAVINVLLGQPVFAEDERNIALAERVIKQFNIHYYLPHPREKYRLAQVNYIDTELIFEDYILQQCQTHKYCVYTYFSSAIINIMNKSDNIEVVALKIDTENPAYDACYDLFDELGVNVIDIRE